SEQUENCE LISTING (76 sequences)

SEQ ID NO: 1 - protein, 495 aa, synthetic construct
  REGION 1..495: note = Murine anti-CD19 CAR;
MASPLTRFLSLNLLLLGESIILGSGEADIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSLQKVNSTTTKPVLRTPSPVHPTGTSQPQRPEDCRPRGSVKGTGLDFACDIYFWALVVVAGVLFCYGLLVTVALCVIWTNSRRNRLLQSDYMNMTPRRPGLTRKPYQPYAPARDFAAYRPKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 2 - DNA, 1485 bp, synthetic construct
  misc_feature 1..1485: note = Murine anti-CD19 CAR;
  source 1..1485: mol_type = other DNA
atggcctcacccctgaccagattcctgtcactgaacctgctgctgctgggcgagtcaatcatcctgggctcaggcgaggccgacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagcctgcagaaggtgaactcaaccaccaccaagcccgtgctgagaaccccctcacccgtgcaccccaccggcacctcacagccccagagacccgaggactgcagacccagaggctcagtgaagggcaccggcctggacttcgcctgcgacatctacttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccaagttctcaagatcagccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctactcagagatcggcaccaagggcgagagaagaagaggcaagggccacgacggcctgtaccagggcctgtcaaccgccaccaaggacacctacgacgccctgcacatgcagaccctggcccccaga

SEQ ID NO: 3 - protein, 488 aa, synthetic construct
  REGION 1..488: note = Human anti-CD19 CAR;
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 1464 bp, synthetic construct
  misc_feature 1..1464: note = Human anti-CD19 CAR;
  source 1..1464: mol_type = other DNA
atggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - protein, 501 aa, synthetic construct
  REGION 1..501: note = Murine anti-MSLN CAR ;
MASPLTRFLSLNLLLLGESIILGSGEAAQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRLQKVNSTTTKPVLRTPSPVHPTGTSQPQRPEDCRPRGSVKGTGLDFACDIYIWAPLAGICVALLLSLIITLIKWIRKKFPHIFKQPFKKTTGAAQEEDACSCRCPQEEEGGGGGYELKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 6 - DNA, 1503 bp, synthetic construct
  misc_feature 1..1503: note = Murine anti-MSLN CAR ;
  source 1..1503: mol_type = other DNA
atggccagccccctgaccagattcctgagcctgaacctgctgctgctgggcgagagcatcatcctgggcagcggcgaggccgcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtccaagaaccagttctccctgaagctgagctctgtgaccgctgcggacacggccgtgtattactgtgcgagatttgactacggtgacttctatgatgcttttgatatctggggccaagggacaatggtcaccgtctcttcaggtggtggtggtagcggcggcggcggctctggtggtggtggatccgaaattgtgttgacgcagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgtctgcagaaggtgaacagcaccaccaccaagcccgtgctgagaacccccagccccgtgcaccccaccggcaccagccagccccagagacccgaggactgcagacccagaggcagcgtgaagggcaccggcctggacttcgcctgcgacatctacatctgggcccccctggccggcatctgcgtggccctgctgctgagcctgatcatcaccctgatcaagtggatcagaaagaagttcccccacatcttcaagcagcccttcaagaagaccaccggcgccgcccaggaggaggacgcctgcagctgcagatgcccccaggaggaggagggcggcggcggcggctacgagctgaagttcagcagaagcgccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctacagcgagatcggcaccaagggcgagagaagaagaggcaagggccacgacggcctgtaccagggcctgagcaccgccaccaaggacacctacgacgccctgcacatgcagaccctggcccccaga

SEQ ID NO: 7 - protein, 485 aa, synthetic construct
  REGION 1..485: note = Human anti-MSLN CAR ;
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - DNA, 1455 bp, synthetic construct
  misc_feature 1..1455: note = Human anti-MSLN CAR ;
  source 1..1455: mol_type = other DNA
atggccctccctgtcaccgccctgctgcttccgctggctcttctgctccacgccgctcggccccaagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 9 - DNA, 738 bp, synthetic construct
  misc_feature 1..738: note = A03 Murine anti-MSLN scFv ;
  source 1..738: mol_type = other DNA
gcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtccaagaaccagttctccctgaagctgagctctgtgaccgctgcggacacggccgtgtattactgtgcgagatttgactacggtgacttctatgatgcttttgatatctggggccaagggacaatggtcaccgtctcttcaggtggtggtggtagcggcggcggcggctctggtggtggtggatccgaaattgtgttgacgcagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 10 - protein, 246 aa, synthetic construct
  REGION 1..246: note = A03 Murine anti-MSLN scFv ;
AQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKR

SEQ ID NO: 11 - DNA, 723 bp, synthetic construct
  misc_feature 1..723: note = M5 Human anti-MSLN scFv ;
  source 1..723: mol_type = other DNA
caagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaag

SEQ ID NO: 12 - protein, 241 aa, synthetic construct
  REGION 1..241: note = M5 Human anti-MSLN scFv ;
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 13 - DNA, 810 bp, synthetic construct
  misc_feature 1..810: note = Anti-GD2 scFv ;
  source 1..810: mol_type = other DNA
atggccttaccagtgaccgccttgctcctgccgctggccttgctgctccacgccgccaggccgggatccgatgttgtgatgacccaaactccactctccctgcctgtcagtcttggagatcaagcctccatctcttgcagatctagtcagagtcttgtacaccgtaacggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacacgttcctccgctcacgttcggtgctgggaccaagctggagctgaaaggaggtggcgggtcagggggtggcggaagcggaggcggcggttcaggcggaggaggctcggaggtgcagcttctgcagtctggacctgagctggagaagccttccgcttcagtgatgatatcctgcaaggcttctggttcctccttcactggctacaacatgaactgggtgaggcagaatattggaaagagccttgaatggattggagctattgatccttactacggtggaactagctacaaccagaagttcaagggcagggccacattgactgtagacaaatcgtccagcacagcctacatgcacctcaagagcctgacatctgaggactctgtctattactgtgtaagcggaatggagtactggggtcaaggaacctcagtcaccgtctcctcatccgga

SEQ ID NO: 14 - protein, 270 aa, synthetic construct
  REGION 1..270: note = Anti-GD2 scFv ;
MALPVTALLLPLALLLHAARPGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSVYYCVSGMEYWGQGTSVTVSSSG

SEQ ID NO: 15 - DNA, 798 bp, synthetic construct
  misc_feature 1..798: note = Anti-HER2 scFv (high affinity) ;
  source 1..798: mol_type = other DNA
atggattttcaggtgcagattttcagcttcctgctaatcagtgcctcagtcataatgtccagaggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctttattctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcg

SEQ ID NO: 16 - protein, 266 aa, synthetic construct
  REGION 1..266: note = Anti-HER2 scFv (high affinity) ;
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 17 - DNA, 798 bp, synthetic construct
  misc_feature 1..798: note = Anti-HER2 scFv (low affinity) ;
  source 1..798: mol_type = other DNA
atggattttcaggtgcagattttcagcttcctgctaatcagtgcctcagtcataatgtccagaggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttcgttgctatggacgtgtggggtcaaggaaccctggtcaccgtctcctcg

SEQ ID NO: 18 - protein, 266 aa, synthetic construct
  REGION 1..266: note = Anti-HER2 scFv (low affinity) ;
MDFQVQIFSFLLISASVIMSRGDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFVAMDVWGQGTLVTVSS

SEQ ID NO: 19 - DNA, 732 bp, synthetic construct
  misc_feature 1..732: note = Anti-TnMuc1 scFv ;
  source 1..732: mol_type = other DNA
caggtgcagctgcagcagtctgatgccgagctcgtgaagcctggcagcagcgtgaagatcagctgcaaggccagcggctacaccttcaccgaccacgccatccactgggtcaagcagaagcctgagcagggcctggagtggatcggccacttcagccccggcaacaccgacatcaagtacaacgacaagttcaagggcaaggccaccctgaccgtggacagaagcagcagcaccgcctacatgcagctgaacagcctgaccagcgaggacagcgccgtgtacttctgcaagaccagcaccttctttttcgactactggggccagggcacaaccctgacagtgtctagcggaggcggaggatctggcggcggaggaagtggcggagggggatctgaactcgtgatgacccagagccccagctctctgacagtgacagccggcgagaaagtgaccatgatctgcaagtcctcccagagcctgctgaactccggcgaccagaagaactacctgacctggtatcagcagaaacccggccagccccccaagctgctgatcttttgggccagcacccgggaaagcggcgtgcccgatagattcacaggcagcggctccggcaccgactttaccctgaccatcagctccgtgcaggccgaggacctggccgtgtattactgccagaacgactacagctaccccctgaccttcggagccggcaccaagctggaactgaag

SEQ ID NO: 20 - protein, 244 aa, synthetic construct
  REGION 1..244: note = Anti-TnMuc1 scFv ;
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 21 - DNA, 792 bp, synthetic construct
  misc_feature 1..792: note = Anti-PSMA scFv ;
  source 1..792: mol_type = other DNA
atggccctgcctgtgacagccctgctgctgcctctggctctgctgctgcacgccgccagacctggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaacgttcagtctgaagacttggcagattatttctgtcagcaatataacagctatcctctcacgttcggtgctgggaccatgctggacctgaaaggaggcggaggatctggcggcggaggaagttctggcggaggcagcgaggtgcagctgcagcagagcggacccgagctcgtgaagcctggaacaagcgtgcggatcagctgcaagaccagcggctacaccttcaccgagtacaccatccactgggtcaagcagtcccacggcaagagcctggagtggatcggcaatatcaaccccaacaacggcggcaccacctacaaccagaagttcgaggacaaggccaccctgaccgtggacaagagcagcagcaccgcctacatggaactgcggagcctgaccagcgaggacagcgccgtgtactattgtgccgccggttggaacttcgactactggggccagggcacaaccctgacagtgtctagcgctagctccgga

SEQ ID NO: 22 - protein, 264 aa, synthetic construct
  REGION 1..264: note = Anti-PSMA scFv ;
MALPVTALLLPLALLLHAARPGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKGGGGSGGGGSSGGGSEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSASSG

SEQ ID NO: 23 - DNA, 801 bp, synthetic construct
  misc_feature 1..801: note = Anti-EGFRvIII scFv ;
  source 1..801: mol_type = other DNA
atggccctccctgtcaccgccctgctgcttccgctggctcttctgctccacgccgctcggcccgagattcagctcgtgcaatcgggagcggaagtcaagaagccaggagagtccttgcggatctcatgcaagggtagcggctttaacatcgaggattactacatccactgggtgaggcagatgccggggaagggactcgaatggatgggacggatcgacccagaaaacgacgaaactaagtacggtccgatcttccaaggccatgtgactattagcgccgatacttcaatcaataccgtgtatctgcaatggtcctcattgaaagcctcagataccgcgatgtactactgtgctttcagaggaggggtctactggggacagggaactaccgtgactgtctcgtccggcggaggcgggtcaggaggtggcggcagcggaggaggagggtccggcggaggtgggtccgacgtcgtgatgacccagagccctgacagcctggcagtgagcctgggcgaaagagctaccattaactgcaaatcgtcgcagagcctgctggactcggacggaaaaacgtacctcaattggctgcagcaaaagcctggccagccaccgaagcgccttatctcactggtgtcgaagctggattcgggagtgcccgatcgcttctccggctcgggatcgggtactgacttcaccctcactatctcctcgcttcaagcagaggacgtggccgtctactactgctggcagggaacccactttccgggaaccttcggcggagggacgaaagtggagatcaag

SEQ ID NO: 24 - protein, 267 aa, synthetic construct
  REGION 1..267: note = Anti-EGFRvIII scFv ;
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 25 - DNA, 729 bp, synthetic construct
  misc_feature 1..729: note = Anti-FAP scFv ;
  source 1..729: mol_type = other DNA
caaattgttctcacccagtctccagcgctcatgtctgcttctccaggggagaaggtcaccatgacctgcactgccagctcaagtgttagttacatgtactggtaccagcagaagccacgatcctcccccaaaccctggatttttctcacctccaacctggcttctggagtccctgctcgcttcagtggccgtgggtctgggacctctttctctctcacaatcagcagcatggaggctgaagatgctgccacttattactgccagcagtggagtggttacccacccatcacattcggctcggggacaaagttggaaataaaaggtggaggtggcagcggaggaggtgggtccggcggtggaggaagccaggtccaactgcagcagcctggggctgaactggtaaagcctggggcttcagtgaagttgtcctgcaaggcgtctggctacaccatcaccagctactctctgcactgggtgaagcagaggcctggacaaggccttgagtggattggagagattaatcctgccaatggtgatcataacttcagtgagaagttcgagatcaaggccacactgactgtagacagctcctccaacacagcattcatgcaactcagcaggctgacatctgaggactctgcggtctattactgtacaagattggacgatagtaggttccactggtacttcgatgtctggggcgcagggaccacggtcaccgtctcctca

SEQ ID NO: 26 - protein, 243 aa, synthetic construct
  REGION 1..243: note = Anti-FAP scFv ;
QIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIKGGGGSGGGGSGGGGSQVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSS

SEQ ID NO: 27 - protein, 27 aa, synthetic construct
  REGION 1..27: note = Mouse CD8 Leader ;
MASPLTRFLSLNLLLLGESIILGSGEA

SEQ ID NO: 28 - DNA, 81 bp, synthetic construct
  misc_feature 1..81: note = Mouse CD8 Leader ;
  source 1..81: mol_type = other DNA
atggcctcacccctgaccagattcctgtcactgaacctgctgctgctgggcgagtcaatcatcctgggctcaggcgaggcc

SEQ ID NO: 29 - protein, 238 aa, synthetic construct
  REGION 1..238: note = Mouse anti-mCD19 scFv ;
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 30 - DNA, 714 bp, synthetic construct
  misc_feature 1..714: note = Mouse anti-mCD19 scFv ;
  source 1..714: mol_type = other DNA
gacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagc

SEQ ID NO: 31 - protein, 51 aa, synthetic construct
  REGION 1..51: note = Mouse CD8 Hinge ;
LQKVNSTTTKPVLRTPSPVHPTGTSQPQRPEDCRPRGSVKGTGLDFACDIY

SEQ ID NO: 32 - DNA, 153 bp, synthetic construct
  misc_feature 1..153: note = Mouse CD8 Hinge ;
  source 1..153: mol_type = other DNA
ctgcagaaggtgaactcaaccaccaccaagcccgtgctgagaaccccctcacccgtgcaccccaccggcacctcacagccccagagacccgaggactgcagacccagaggctcagtgaagggcaccggcctggacttcgcctgcgacatctac

SEQ ID NO: 33 - protein, 27 aa, synthetic construct
  REGION 1..27: note = Mouse CD28 TM ;
FWALVVVAGVLFCYGLLVTVALCVIWT

SEQ ID NO: 34 - DNA, 81 bp, synthetic construct
  misc_feature 1..81: note = Mouse CD28 TM ;
  source 1..81: mol_type = other DNA
ttttgggcactggtcgtggttgctggagtcctgttttgttatggcttgctagtgacagtggctctttgtgttatctggaca

SEQ ID NO: 35 - protein, 41 aa, synthetic construct
  REGION 1..41: note = Mouse CD28 ICD ;
NSRRNRLLQSDYMNMTPRRPGLTRKPYQPYAPARDFAAYRP

SEQ ID NO: 36 - DNA, 123 bp, synthetic construct
  misc_feature 1..123: note = Mouse CD28 ICD ;
  source 1..123: mol_type = other DNA
aatagtagaaggaacagactccttcaaagtgactacatgaacatgactccccggaggcctgggctcactcgaaagccttaccagccctacgcccctgccagagactttgcagcgtaccgcccc

SEQ ID NO: 37 - protein, 111 aa, synthetic construct
  REGION 1..111: note = Mouse CD3z ;
KFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 38 - DNA, 333 bp, synthetic construct
  misc_feature 1..333: note = Mouse CD3z ;
  source 1..333: mol_type = other DNA
aagttctcaagatcagccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctactcagagatcggcaccaagggcgagagaagaagaggcaagggccacgacggcctgtaccagggcctgtcaaccgccaccaaggacacctacgacgccctgcacatgcagaccctggcccccaga

SEQ ID NO: 39 - protein, 21 aa, synthetic construct
  REGION 1..21: note = Human CD8 leader ;
MALPVTALLLPLALLLHAARP

SEQ ID NO: 40 - DNA, 63 bp, synthetic construct
  misc_feature 1..63: note = Human CD8 leader ;
  source 1..63: mol_type = other DNA
atggccttaccagtgaccgccttgctcctgccgctggccttgctgctccacgccgccaggccg

SEQ ID NO: 41 - protein, 242 aa, synthetic construct
  REGION 1..242: note = Human anti-CD19 scFv ;
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 42 - DNA, 726 bp, synthetic construct
  misc_feature 1..726: note = Human anti-CD19 scFv ;
  source 1..726: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 43 - protein, 45 aa, synthetic construct
  REGION 1..45: note = Human CD8 Hinge ;
TTTPAPRPPTPAPTIASQPLSLRPEACRPAAGGAVHTRGLDFACD

SEQ ID NO: 44 - DNA, 135 bp, synthetic construct
  misc_feature 1..135: note = Human CD8 Hinge ;
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 45 - protein, 27 aa, synthetic construct
  REGION 1..27: note = Human CD28 TM ;
FWVLVVVGGVLACYSLLVTVAFIIFWV

SEQ ID NO: 46 - DNA, 81 bp, synthetic construct
  misc_feature 1..81: note = Human CD28 TM ;
  source 1..81: mol_type = other DNA
ttttgggtgctggtggtggttggtggagtcctggcttgctatagcttgctagtaacagtggcctttattattttctgggtg

SEQ ID NO: 47 - protein, 41 aa, synthetic construct
  REGION 1..41: note = Human CD28 ICD ;
RSKRSRLLHSDYMNMTPRRPGPTRKHYQPYAPPRDFAAYRS

SEQ ID NO: 48 - DNA, 123 bp, synthetic construct
  misc_feature 1..123: note = Human CD28 ICD ;
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 49 - protein, 112 aa, synthetic construct
  REGION 1..112: note = Human CD3z ICD ;
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - DNA, 336 bp, synthetic construct
  misc_feature 1..336: note = Human CD3z ICD ;
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 51 - protein, 27 aa, synthetic construct
  REGION 1..27: note = Mouse CD8 Leader ;
MASPLTRFLSLNLLLLGESIILGSGEA

SEQ ID NO: 52 - DNA, 81 bp, synthetic construct
  misc_feature 1..81: note = Mouse CD8 Leader ;
  source 1..81: mol_type = other DNA
atggccagccccctgaccagattcctgagcctgaacctgctgctgctgggcgagagcatcatcctgggcagcggcgaggcc

SEQ ID NO: 53 - protein, 246 aa, synthetic construct; identical to SEQ ID NO: 10
  REGION 1..246: note = A03 mouse anti-Meso scFv ;

SEQ ID NO: 54 - DNA, 738 bp, synthetic construct; identical to SEQ ID NO: 9
  misc_feature 1..738: note = A03 mouse anti-Meso scFv ;
  source 1..738: mol_type = other DNA

SEQ ID NO: 55 - protein, 51 aa, synthetic construct; identical to SEQ ID NO: 31
  REGION 1..51: note = Mouse CD8 Hinge ;

SEQ ID NO: 56 - DNA, 153 bp, synthetic construct
  misc_feature 1..153: note = Mouse CD8 Hinge;
  source 1..153: mol_type = other DNA
ctgcagaaggtgaacagcaccaccaccaagcccgtgctgagaacccccagccccgtgcaccccaccggcaccagccagccccagagacccgaggactgcagacccagaggcagcgtgaagggcaccggcctggacttcgcctgcgacatctac

SEQ ID NO: 57 - protein, 21 aa, synthetic construct
  REGION 1..21: note = Mouse CD8 TM ;
IWAPLAGICVALLLSLIITLI

SEQ ID NO: 58 - DNA, 63 bp, synthetic construct
  misc_feature 1..63: note = Mouse CD8 TM ;
  source 1..63: mol_type = other DNA
atctgggcccccctggccggcatctgcgtggccctgctgctgagcctgatcatcaccctgatc

SEQ ID NO: 59 - protein, 45 aa, synthetic construct
  REGION 1..45: note = Mouse 4-1BB ICD ;
KWIRKKFPHIFKQPFKKTTGAAQEEDACSCRCPQEEEGGGGGYEL

SEQ ID NO: 60 - DNA, 135 bp, synthetic construct
  misc_feature 1..135: note = Mouse 4-1BB ICD ;
  source 1..135: mol_type = other DNA
aagtggatcagaaagaagttcccccacatcttcaagcagcccttcaagaagaccaccggcgccgcccaggaggaggacgcctgcagctgcagatgcccccaggaggaggagggcggcggcggcggctacgagctg

SEQ ID NO: 61 - protein, 111 aa, synthetic construct; identical to SEQ ID NO: 37
  REGION 1..111: note = Mouse CD3z ICD ;

SEQ ID NO: 62 - DNA, 333 bp, synthetic construct
  misc_feature 1..333: note = Mouse CD3z ICD ;
  source 1..333: mol_type = other DNA
aagttcagcagaagcgccgagaccgccgccaacctgcaggaccccaaccagctgtacaacgagctgaacctgggcagaagagaggagtacgacgtgctggagaagaagagagccagagaccccgagatgggcggcaagcagcagagaagaagaaacccccaggagggcgtgtacaacgccctgcagaaggacaagatggccgaggcctacagcgagatcggcaccaagggcgagagaagaagaggcaagggccacgacggcctgtaccagggcctgagcaccgccaccaaggacacctacgacgccctgcacatgcagaccctggcccccaga

SEQ ID NO: 63 - protein, 21 aa, synthetic construct
  REGION 1..21: note = Human CD8 Leader ;
MALPVTALLLPLALLLHAARP

SEQ ID NO: 64 - DNA, 63 bp, synthetic construct
  misc_feature 1..63: note = Human CD8 Leader ;
  source 1..63: mol_type = other DNA
atggccctccctgtcaccgccctgctgcttccgctggctcttctgctccacgccgctcggccc

SEQ ID NO: 65 - protein, 241 aa, synthetic construct; identical to SEQ ID NO: 12
  REGION 1..241: note = Human M5 scFv ;

SEQ ID NO: 66 - DNA, 723 bp, synthetic construct; identical to SEQ ID NO: 11
  misc_feature 1..723: note = Human M5 scFv ;
  source 1..723: mol_type = other DNA

SEQ ID NO: 67 - protein, 45 aa, synthetic construct; identical to SEQ ID NO: 43
  REGION 1..45: note = Human CD8 Hinge ;

SEQ ID NO: 68 - DNA, 135 bp, synthetic construct
  misc_feature 1..135: note = Human CD8 Hinge ;
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 69 - protein, 24 aa, synthetic construct
  REGION 1..24: note = Human CD8 TM ;
IYIWAPLAGTCGVLLLSLVITLYC

SEQ ID NO: 70 - DNA, 72 bp, synthetic construct
  misc_feature 1..72: note = Human CD8 TM ;
  source 1..72: mol_type = other DNA
atctacatttgggcccctctggctggtacttgcggggtcctgctgctttcactcgtgatcactctttactgt

SEQ ID NO: 71 - protein, 42 aa, synthetic construct
  REGION 1..42: note = Human 4-1BB ICD ;
KRGRKKLLYIFKQPFMRPVQTTQEEDGCSCRFPEEEEGGCEL

SEQ ID NO: 72 - DNA, 126 bp, synthetic construct
  misc_feature 1..126: note = Human 4-1BB ICD ;
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 73 - protein, 112 aa, synthetic construct
  REGION 1..112: note = Human CD3z ICD ;
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 336 bp, synthetic construct
  misc_feature 1..336: note = Human CD3z ICD ;
  source 1..336: mol_type = other DNA
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 75 - DNA, 885 bp, synthetic construct
  misc_feature 1..885: note = CD19 antigen ;
  source 1..885: mol_type = other DNA
aggccccagaagtccttactggtggaggtagaagagggaggcaatgttgtgctgccatgcctcccggactcctcacctgtctcttctgagaagctggcttggtatcgaggtaaccagtcaacacccttcctggagctgagccccgggtcccctggcctgggattgcacgtggggtccctgggcatcttgctagtgattgtcaatgtctcagaccatatggggggcttctacctgtgccagaagaggccccctttcaaggacatctggcagcctgcctggacagtgaacgtggaggatagtggggagatgttccggtggaatgcttcagacgtcagggacctggactgtgacctaaggaacaggtcctctgggagccacaggtccacttctggttcccagctgtatgtgtgggctaaagaccatcctaaggtctggggaacaaagcctgtatgtgcccctcgggggagcagtttgaatcagagtctaatcaaccaagatctcactgtggcacccggctccacactttggctgtcctgtggggtaccccctgtcccagtggccaaaggctccatctcctggacccatgtgcatcctaggagacctaatgtttcactactgagcctaagccttgggggagagcacccggtcagagagatgtgggtttgggggtctcttctgcttctgccccaagccacagctttagatgaaggcacctattattgtctccgaggaaacctgaccatcgagaggcacgtgaaggtcattgcaaggtcagcagtgtggctctggctgttgagaactggtggatggatagtcccagtggtgactttagtatatgtcatcttctgtatggtttctctggtggcttttctctattgtcaaaga

SEQ ID NO: 76 - protein, 295 aa, synthetic construct
  REGION 1..295: note = CD19 antigen ;
RPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKGSISWTHVHPRRPNVSLLSLSLGGEHPVREMWVWGSLLLLPQATALDEGTYYCLRGNLTIERHVKVIARSAVWLWLLRTGGWIVPVVTLVYVIFCMVSLVAFLYCQR

(19) United States
(12) Patent Application Publication
(10) Pub. No.: US 2025/0177442 A1
(43) Pub. Date: Jun. 5, 2025

(54) BOOSTING CHIMERIC ANTIGEN RECEPTOR CELLS IN THE BLOOD

(71) Applicant: The Trustees of the University of Pennsylvania, Philadelphia, PA (US)

(72) Inventors: Carl H. June, Merion Station, PA (US); Regina M. Young, Bryn Mawr, PA (US); Ziming Wang, Philadelphia, PA (US); Mikko Siurala, Philadelphia, PA (US); Amanda Finck, Philadelphia, PA (US)

(21) Appl. No.: 18/837,734

(22) PCT Filed: Feb. 13, 2023
(86) PCT No.: PCT/US2023/062475

§ 371 Date: Aug. 12, 2024

Related U.S. Application Data
(60) Provisional application No. 63/310,355, filed on Feb. 15, 2022.

(51) Int. Cl. A61K 35/17; A61K 39/00; A61K 40/11; A61K 40/31; A61K 40/42; A61P 35/00; C07K 14/71; C07K 16/28; C12N 15/86

(52) U.S. Cl. CPC A61K 35/17; A61K 39/001112; A61K 40/11; A61K 40/31; A61K 40/4203; A61K 40/4211; A61P 35/00; C07K 14/71; C07K 16/2803; C12N 15/86; A61K 2239/13; A61K 2239/21; A61K 2239/22; A61K 2239/23; A61K 2239/29; C12N 2740/15043; C12N 2750/14143

(57) ABSTRACT

The present disclosure provides modified cell(s), i.e., immune cell(s) or precursor cell(s) thereof, wherein the cell(s) are engineered to express: (a) a first chimeric antigen receptor (CAR) having affinity for CD19, and (b) a second CAR having affinity for a tumor antigen, wherein the tumor antigen is not CD19. Also provided are methods and uses of the modified cells, e.g., for treating at least one sign and/or symptom of cancer in a subject. The modified cells expand in the peripheral blood of the subject. Related nucleic acids, vectors, and pharmaceutical compositions are also provided.

### CROSS-REFERENCE TO RELATED APPLICATION

The current application claims priority under 35 U.S.C. § 119(e) to U.S. Provisional Patent Application No. 63/310,355, filed Feb. 15, 2022, which is hereby incorporated by reference in its entirety herein.

### BACKGROUND OF THE INVENTION

Chimeric Antigen Receptor T-cell (CART) immunotherapy has shown significant improvement in the clinical outcomes of patient with B cell malignancies, including relapsed/refractory (r/r) lymphomas and leukemias (Lee, et al., The Lancet (2015) 385(9967):517-28; Maude et al., New England Journal of Medicine (2018) 378(5):439-48; Park et al., New England Journal of Medicine (2018) 378(5):449-59; Schuster, et al., New England Journal of Medicine (2019) 380(1):45-56; Turtle et al., Science Translational Medicine (2016) 8(355):355ra116-355ra116). Despite the remarkable clinical results of anti-CD19 CART (CART19), the vast majority of patients treated with the currently-approved CART products have failed these treatments. Analyses of acute lymphoblastic leukemia (ALL) and chronic lymphoblastic leukemia (CLL) patient samples from these trials have revealed that the degree of response to CART19 therapy is correlated with high levels of CAR T expansion in the blood and acquisition and maintenance of B cell aplasia. Unfortunately, patient responses to CAR T cell therapy designed to treat solid tumors have been less efficacious. CAR T cells directed against solid tumor targets do not typically encounter their cognate target in the blood, but instead undergo limited homeostatic CAR T cell expansion in the blood and traffic to tumor sites. The number of CAR T cells that reach the tumor are insufficient to eradicate disease. Furthermore increasing the infusion dose of CAR T cells has revealed on-target off-tumor toxicity. There is a need in the art for enhancing anti-tumor efficacy of CAR adoptive cell therapies in order to improve the clinical outcome of patients. The present invention addresses this need.

### SUMMARY OF THE DISCLOSURE

The present invention includes compositions and methods comprising modified cells, e.g., for treating at least one sign and/or symptom of cancer in a subject. The modified cells expand in the peripheral blood of the subject. Related nucleic acids, vectors, and pharmaceutical compositions are also provided.

As such, in one aspect, the invention provides an isolated nucleic acid comprising:
a. a first nucleotide sequence encoding a first chimeric antigen receptor (CAR) comprising:
   (i) a first extracellular antigen binding domain having affinity for CD19,
   (ii) a first transmembrane domain, and
   (iii) a first intracellular domain comprising at least one costimulatory domain and a signaling domain; and
b. a second nucleotide sequence encoding a second CAR comprising:
   (i) a second extracellular antigen binding domain having affinity for a tumor antigen, wherein the tumor antigen is not CD19,
   (ii) a second transmembrane domain, and
   (iii) a second intracellular domain comprising at least one costimulatory domain and a signaling domain.

In certain embodiments, the first nucleotide sequence is situated 5′ to the second nucleotide sequence.

In certain embodiments, the first nucleotide sequence is situated 3′ to the second nucleotide sequence.

In certain embodiments, the isolated nucleic acid further comprises a linker nucleotide sequence situated between the first nucleotide sequence and the second nucleotide sequence, wherein the linker nucleotide sequence comprises a ribosome slip sequence selected from the group consisting of P2A, T2A, E2A, and F2A.

In certain embodiments, the isolated nucleic acid further comprises a promoter operably linked to the first nucleotide sequence and/or to the second nucleotide sequence.

In certain embodiments, the promoter is an EF1α promoter.

In certain embodiments, the isolated nucleic acid further comprises a posttranscriptional regulatory element.

In certain embodiments, the posttranscriptional regulatory element is a woodchuck hepatitis virus (WHV) posttranscriptional regulatory element (WPRE).

In certain embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), Glycosyl-phosphatidylinositol (GPI)-linked GDNF family a-receptor 4 (GFRα4; GFRalpha4), HER2, HLA-A2, ICAM1, interleukin 13 receptor subunit alpha (IL3Rα), interleukin 13 receptor subunit alpha 1 (IL13Rα1), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), VEGFR2, and any combination thereof.

In certain embodiments, the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family a-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

In certain embodiments, the first extracellular antigen binding domain and the second extracellular antigen binding domain are each independently selected from a single-chain variable fragment (scFv) and an Fab.

In certain embodiments, the at least one costimulatory domain of the first or second intracellular domain comprises a costimulatory domain of a protein independently selected from 4-1BB, CD2, CD28, and ICOS.

In certain embodiments, the signaling domain comprises a CD3 zeta signaling domain.

In certain embodiments, the first and/or second transmembrane domain comprises a transmembrane domain of a protein independently selected from CD8, CD28, and 4-1BB.

In certain embodiments, the first and/or second CAR further comprises a hinge domain.

In certain embodiments, the hinge domain comprises a CD8 hinge domain.

In certain embodiments, the invention provides the isolated nucleic acid disclosed herein, wherein:
a. the first CAR comprises:
   (i) a first extracellular antigen binding domain comprising an anti-CD19 scFv,
   (ii) a first transmembrane domain, and
   (iii) a first intracellular domain comprising a CD28 costimulatory domain and a CD3 zeta signaling domain; and
b. the second CAR comprises:
   (i) a second extracellular antigen binding domain comprising an anti-tumor antigen scFv, wherein the tumor antigen is not CD19,
   (ii) a second transmembrane domain, and
   (iii) a second intracellular domain comprising a 4-1BB costimulatory domain and a CD3 zeta signaling domain.

In certain embodiments, the invention provides the isolated nucleic acid disclosed herein, wherein:
a. the first CAR consists essentially of
   (i) a CD8 leader sequence;
   (ii) a first extracellular antigen binding domain comprising an anti-CD19 scFv,
   (iii) a CD8 hinge;
   (iv) a CD28 transmembrane domain, and
   (v) a first intracellular domain comprising a CD28 costimulatory domain and a CD3 zeta signaling domain; and
b. the second CAR consists essentially of
   (i) a CD8 leader sequence;
   (ii) a second extracellular antigen binding domain comprising an anti-mesothelin scFv,
   (iii) a CD8 hinge;
   (iv) a CD8 transmembrane domain, and
   (v) a second intracellular domain comprising a 4-1BB costimulatory domain and a CD3 zeta signaling domain.

In certain embodiments, the isolated nucleic acid further comprises a third nucleotide sequence encoding a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R.

In certain embodiments, the isolated nucleic acid further comprises a fourth nucleotide sequence encoding a dominant negative receptor, wherein the dominant negative receptor is dnTGFβR.

In one aspect, the invention provides a vector comprising the isolated nucleic acid disclosed herein.

In certain embodiments, the vector is a lentiviral vector or a retroviral vector.

In one aspect, the invention provides a modified cell comprising the isolated nucleic acid disclosed herein or the vector disclosed herein.

In certain embodiments, the cell is selected from a bacterial cell, a fungal cell, a yeast cell, an insect cell, an animal cell, a mammalian cell, and a human cell.

In certain embodiments, the cell is a mammalian cell or a human cell, and the cell is an immune cell or precursor cell thereof.

In certain embodiments, the immune cell is a T cell.

In certain embodiments, the cell is an immune cell or precursor cell thereof, and the cell is engineered to express:
a. a first chimeric antigen receptor (CAR) comprising:
   (i) a first extracellular antigen binding domain having affinity for CD19,
   (ii) a first transmembrane domain, and
   (iii) a first intracellular domain comprising at least one costimulatory domain and a signaling domain; and
b. a second CAR comprising:
   (i) a second extracellular antigen binding domain having affinity for a tumor antigen, wherein the tumor antigen is not CD19,
   (ii) a second transmembrane domain, and
   (iii) a second intracellular domain comprising at least one costimulatory domain and a signaling domain.

In certain embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), HER2, HLA-A2, ICAM1, interleukin 13 receptor subunit alpha (IL3Rα), interleukin 13 receptor subunit alpha 1 (IL13Rα1), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), Glycosyl-phosphatidylinositol (GPI)-linked GDNF family α-receptor 4 (GFRα4; GFRalpha4), VEGFR2, and any combination thereof.

In certain embodiments, the tumor antigen is selected from the group consisting of prostate specific membrane antigen (PSMA), MUC1, Tn-glycoform of MUC1 (TnMUC1), folate receptor alpha (FRα), mesothelin, New York esophageal squamous cell carcinoma-1 (NY-ESO-1), glypican 2 (GPC2), Glycosyl-phosphatidylinositol (GPI)-linked GDNF family α-receptor 4 (GFRα4; GFRalpha4), prostate stem cell antigen (PSCA), fibroblast activation protein (FAP), epidermal growth factor receptor (EGFR), and interleukin 13 receptor subunit alpha 2 (IL13Rα2).

In certain embodiments, the first extracellular antigen binding domain and the second extracellular antigen binding domain are each independently selected from a single-chain variable fragment (scFv) and an Fab.

In certain embodiments, the at least one costimulatory domain of the first or second intracellular domain comprises a costimulatory domain of a protein independently selected from 4-1BB, CD2, CD28, and ICOS.

In certain embodiments, the signaling domain comprises a CD3 zeta signaling domain.

In certain embodiments, the first and/or second transmembrane domain comprises a transmembrane domain of a protein independently selected from CD8, CD28, and 4-1BB.

In certain embodiments, the first and/or second CAR further comprises a hinge domain.

In certain embodiments, the hinge domain comprises a CD8 hinge domain.

In certain embodiments, the invention provides the modified cell disclosed herein, wherein:
a. the first CAR comprises:
   (i) a first extracellular antigen binding domain comprising an anti-CD19 scFv,
   (ii) a first transmembrane domain, and
   (iii) a first intracellular domain comprising a CD28 costimulatory domain and a CD3 zeta signaling domain; and
b. the second CAR comprises:
   (i) a second extracellular antigen binding domain comprising an anti-tumor antigen scFv, wherein the tumor antigen is not CD19,
   (ii) a second transmembrane domain, and
   (iii) a second intracellular domain comprising a 4-1BB costimulatory domain and a CD3 zeta signaling domain.

In certain embodiments, the invention provides the modified cell disclosed herein, wherein:
a. the first CAR consists essentially of
   (i) a CD8 leader sequence;
   (ii) a first extracellular antigen binding domain comprising an anti-CD19 scFv,
   (iii) a CD8 hinge;
   (iv) a CD28 transmembrane domain, and
   (v) a first intracellular domain comprising a CD28 costimulatory domain and a CD3 zeta signaling domain; and
b. the second CAR consists essentially of
   (i) a CD8 leader sequence;
   (ii) a second extracellular antigen binding domain comprising an anti-mesothelin scFv,
   (iii) a CD8 hinge;
   (iv) a CD8 transmembrane domain, and
   (v) a second intracellular domain comprising a 4-1BB costimulatory domain and a CD3 zeta signaling domain.

In certain embodiments, the cell is further engineered to express a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R.

In certain embodiments, the cell is further engineered to express a dominant negative receptor, wherein the dominant negative receptor is dnTGFβR.

In certain embodiments, the cell comprises a vector encoding the first CAR and the second CAR.

In certain embodiments, the vector is a lentiviral vector or a retroviral vector.

In certain embodiments, the vector further encodes the switch receptor disclosed herein and/or the dominant negative receptor disclosed herein.

In certain embodiments, the cell is a mouse cell or a human cell.

In certain embodiments, the cell is a T cell.

In one aspect, the invention provides a pharmaceutical composition comprising a population of the modified cell disclosed herein and at least one pharmaceutically acceptable carrier.

In one aspect, the invention provides a method of treating cancer in a subject in need thereof, the method comprising administering to the subject the pharmaceutical composition disclosed herein.

In one aspect, the invention provides a method of treating cancer in a subject in need thereof, the method comprising administering to the subject a population of modified cells, wherein the cells are immune cells or precursor cells thereof, and wherein the cells are engineered to express:
a. a first chimeric antigen receptor (CAR) comprising:
   (i) a first extracellular antigen binding domain having affinity for CD19,
   (ii) a first transmembrane domain, and
   (iii) a first intracellular domain comprising at least one costimulatory domain and a signaling domain; and
b. a second CAR comprising:
   (i) a second extracellular antigen binding domain having affinity for a tumor antigen, wherein the tumor antigen is not CD19,
   (ii) a second transmembrane domain, and
   (iii) a second intracellular domain comprising at least one costimulatory domain and a signaling domain.

In certain embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), HER2, HLA-A2, ICAM1, interleukin 13 receptor subunit alpha (IL3Rα), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), Glycosyl-phosphatidylinositol (GPI)-linked GDNF family α-receptor 4 (GFRα4; GFRalpha4), VEGFR2, and any combination thereof.

In certain embodiments, the tumor antigen is selected from the group consisting of prostate specific membrane antigen (PSMA), MUC1, Tn-glycoform of MUC1 (TnMUC1), folate receptor alpha (FRα), mesothelin, New York esophageal squamous cell carcinoma-1 (NY-ESO-1), glypican 2 (GPC2), Glycosyl-phosphatidylinositol (GPI)-linked GDNF family α-receptor 4 (GFRα4; GFRalpha4), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

In certain embodiments, the first extracellular antigen binding domain and the second extracellular antigen binding domain are each independently selected from a single-chain variable fragment (scFv) and an Fab.

In certain embodiments, the at least one costimulatory domain of the first or second intracellular domain comprises a costimulatory domain of a protein independently selected from 4-1BB, CD2, CD28, and ICOS.

In certain embodiments, the signaling domain comprises a CD3 zeta signaling domain.

In certain embodiments, the first and/or second transmembrane domain comprises a transmembrane domain of a protein independently selected from CD8, CD28, and 4-1BB.

In certain embodiments, the first and/or second CAR further comprises a hinge domain.

In certain embodiments, the hinge domain comprises a CD8 hinge domain.

In certain embodiments, the invention provides the method disclosed herein, wherein:
a. the first CAR comprises:
   (i) a first extracellular antigen binding domain comprising an anti-CD19 scFv,
   (ii) a first transmembrane domain, and
   (iii) a first intracellular domain comprising a CD28 costimulatory domain and a CD3 zeta signaling domain; and
b. the second CAR comprises:
   (i) a second extracellular antigen binding domain comprising an anti-tumor antigen scFv, wherein the tumor antigen is not CD19,
   (ii) a second transmembrane domain, and
   (iii) a second intracellular domain comprising a 4-1BB costimulatory domain and a CD3 zeta signaling domain.

In certain embodiments, the invention provides the method disclosed herein, wherein:
a. the first CAR consists essentially of:
   (i) a CD8 leader sequence;
   (ii) a first extracellular antigen binding domain comprising an anti-CD19 scFv,
   (iii) a CD8 hinge;
   (iv) a CD28 transmembrane domain, and
   (v) a first intracellular domain comprising a CD28 costimulatory domain and a CD3 zeta signaling domain; and
b. the second CAR consists essentially of:
   (i) a CD8 leader sequence;
   (ii) a second extracellular antigen binding domain comprising an anti-mesothelin scFv,
   (iii) a CD8 hinge;
   (iv) a CD8 transmembrane domain, and
   (v) a second intracellular domain comprising a 4-1BB costimulatory domain and a CD3 zeta signaling domain.

In certain embodiments, the modified cells are further engineered to express a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R.

In certain embodiments, the modified cells are further engineered to express a dominant negative receptor, wherein the dominant negative receptor is dnTGFβR.

In certain embodiments, the modified cells comprise a vector encoding the first CAR and the second CAR.

In certain embodiments, the vector is a lentiviral vector or a retroviral vector.

In certain embodiments, the vector further encodes the switch receptor of embodiment 57 and/or the dominant negative receptor of embodiment 58.

In certain embodiments, the modified cells are mouse cells or human cells.

In certain embodiments, the population of modified cells comprises T cells.

In certain embodiments, the modified cells are autologous to the subject.

In certain embodiments, the modified cells are allogeneic to the subject.

In certain embodiments, the population of modified cells are administered as a pharmaceutical composition comprising the population of modified cells and at least one pharmaceutically acceptable carrier.

In certain embodiments, the population of modified cells comprises about 1×10⁶ to about 1×10⁹ cells.

In certain embodiments, the modified cells exhibit expansion in peripheral blood of the subject.

In certain embodiments, the expansion is at least 10-fold, at least 100-fold, or at least 1000-fold.

In certain embodiments, the modified cells are detectable for at least 24 months after administering the cells.

In certain embodiments, the subject is a human.

In certain embodiments, the cancer is selected from breast cancer, prostate cancer, ovarian cancer, cervical cancer, skin cancer, prostate cancer, pancreatic cancer, colorectal cancer, renal cancer, liver cancer, brain cancer, lymphoma, leukemia, lung cancer, and thyroid cancer.

In certain embodiments, the population of modified cells comprises T cells and at least 30% or at least 40% of the population of modified cells at day 7 post-administration or beyond are phenotypically central memory T cells.

In certain embodiments, the method further comprises administering a CD19 antigen to the subject.

In certain embodiments, administering the CD19 antigen comprises administering a vector encoding the CD19 antigen or a cell engineered to express the CD19 antigen.

In certain embodiments, the CD19 antigen comprises a CD19 extracellular domain or antigenic fragment thereof.

In certain embodiments, the CD19 antigen is administered prior to, concurrently with, or after the administration of the population of modified cells.

In certain embodiments, the vector encoding the CD19 antigen is an adenoviral vector.

In certain embodiments, the method further comprises administering an anti-PD1 immunotherapy to the subject.

In certain embodiments, the anti-PD1 immunotherapy is an anti-PD1 antibody.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A-FIG. 1B illustrate design and expression of two pTRPE lentiviral constructs for expression of dual boost CARs in the same cell. One CAR comprises an anti-human CD19 scFv, a CD28 costimulatory domain and a CD3 zeta signaling domain (hCD19/28z) for boosting the CAR T cell in peripheral blood (PB). The other CAR comprises an scFv targeting a tumor antigen (here, the example is M5, an anti-human mesothelin (MSLN) scFv), and further comprises a 4-1BB costimulatory domain and a CD3 zeta signaling domain (M5BBZ). FIG. 1A provides schematatics of the pTRPE-hCD19/28z-M5BBZ and pTRPE-M5BBZ-hCD19/28z dual CAR constructs. FIG. 1B provides flow cytometry data illustrating expression of the hCD19/28z and M5BBZ CARs in T cells from the lentiviral constructs.

FIG. 2A-FIG. 2B provide data illustrating expansion of dual CAR T cells when co-cultured with irradiated Nalm6 human tumor cell line (which expresses CD19) in vitro. FIG. 2A provides a graph illustrating the number of live cells after four days co-culture with irradiated CD19+Nalm6 compared to without irradiated Nalm6 for T cells transduced with the indicated constructs. UTD, untransduced. FIG. 2B provides CSFE staining data illustrating similar CAR T expansion in CD4+ and CD8+ cells for T cell transduced with either dual CAR construct (pTRPE-M5BBZ-hCD19/28z or pTRPE-hCD19/28z-M5BBz) or with a single anti-CD19 CAR construct (pNVS-hCD19BBZ) but not for T cells transduced with the single M5 CAR or untransduced (UTD) T cells.

FIG. 3A-FIG. 3B provide data related to cytotoxicity of CAR T cells for mesothelin-positive pancreatic tumor AsPC1 cells in vitro. FIG. 3A illustrates the finding that T cells transduced with the pTRPE-hCD19/28z-M5BBZ construct or the pTRPE-M5BBZ-hCD19/28z construct kill AsPC1 cells. FIG. 3B illustrates the finding that co-culture of the dual CART cells with irradiated Nalm6 cells accelerates the AsPC1 cell killing by dual CART cells The enhancement of AsPC1 cell killing was higher for T cells transduced with the pTRPE-hCD19/28z-M5BBZ construct compared to T cells transduced with pTRPE-M5BBZ-hCD19/28z.

FIG. 4A-FIG. 4D provide data related to cytotoxicity of CART cells for mesothelin-positive A549 cells derived from type 2 pneumocytes which serve as a model for measuring potential on-target off-tumor toxicity in lung tissue. FIG. 4A and FIG. 4B each provides a graph illustrating the finding that T cells comprising the pTRPE-hCD19/28z-M5BBZ dual CAR construct exhibit reduced cytotoxicity of A549 cells compared to T cells transduced with the pTRPE-M5BBZ single CAR construct or the pTRPE-M5BBZ-hCD19/28z dual CAR construct. FIG. 4C illustrates the finding that, after co-culture with irradiated Nalm6 cells, T cells transduced with the pTRPE-hCD19/28z-M5BBZ dual CAR construct exhibit reduced cytotoxicity of A549 cells compared to T cells comprising the pTRPE-M5BBZ-hCD19/28z dual CAR construct. FIG. 4D provides data illustrating the finding that T cells transduced with the pTRPE-hCD19/28z-M5BBZ dual CAR construct express lower levels of the M5BBZ CAR compared to T cells transduced with the pTRPE-M5BBZ-hCD19/28z dual CAR construct.

FIG. 5A-FIG. 5F provide data illustrating the finding that irradiated NALM6 stimulates dual CAR T cell expansion in tumor-free NSG mice in vivo. FIG. 5A is a schematic of the experiment. FIG. 5B provides data illustrating expansion of T cells transduced with either the pTRPE-hCD19/28z-M5BBZ dual CAR construct or the pTRPE-M5BBZ-hCD19/28z dual CAR construct in vivo. FIG. 5C provides data illustrating that T cells transduced with the pTRPE-M5BBZ construct did not expand. FIG. 5D provides data illustrating dual CAR T expansion in various organs. FIG. 5E provides data illustrating a significant increase in the secretion of cytotoxic cytokines (interferon production regulator (IFNr), perform, granzyme A, and granulysin) in the serum. FIG. 5F provides data related to phenotypes of T cells harvested at Day 20.

FIG. 6A-FIG. 6D provide data illustrating the finding that irradiated NALM6 enhances dual CAR T cell killing of AsPC1 pancreatic tumors in NSG mice in vivo. FIG. 6A is a schematic of the experiment for mice not administered irradiated Nalm6. FIG. 6B provides a graph plotting tumor size versus day post T cell injection illustrating the finding that T cells transduced with the pTRPE-hCD19/28z-M5BBZ dual CAR construct display significantly enhanced pancreatic tumor killing compared to T cells transduced with the pTRPE-M5BBZ single CAR construct. FIG. 6C provides a graph plotting tumor size versus day post T cell injection illustrating the finding that irradiated NALM6 enhances and accelerates dual CAR T cell killing of AsPC1 pancreatic tumors in NSG mice in vivo. FIG. 6D provides data illustrating that the enhancement of pancreatic tumor killing in vivo for T cells transduced with the pTRPE-hCD19/28z-M5BBZ dual CAR construct compared to the pTRPE-M5BBZ single CAR construct is correlated with higher CAR T numbers in the tumors and the shift of CAR T cells to CD4+ cells for T cells transduced with the dual CAR construct.

FIG. 7A-FIG. 7E provide data related to murine dual CAR cytotoxicity in syngeneic mice. FIG. 7A provides a graph illustrating CAR T cell killing of mesothelin-positive PDA7940bWT cell line in vitro for murine T cells transduced with the indicated constructs. FIG. 7B provides data illustrating a decrease of CD19+ cells and an increase of CD45.1+ cells in mice administered murine T cells transduced with the murine dual CAR construct, but not either single CAR construct, without lymph-depletion. FIG. 7C provides data illustrating that the expanded dual CAR T cells are mainly in spleen compared to liver. FIG. 7D provides data related to phenotypes of T cells in the blood harvested on Day 7. FIG. 7E provides data related to phenotypes of T cells in the spleen harvested on Day 7.

FIG. 8 is an experimental design schematic. C57BL/6 syngeneic mice were implanted with PDA (pancreatic) tumors at day −7. On day 0, 1e6 CD45.1+ CAR T cells (transduced with the MSGV-anti-moCD19-MuCD28z-anti-moMeso-A03-3-MuBBz dual CAR construct or with the MSGV-anti-moMeso-A03-3-MuBBz single CAR construct) were infused into the mice. The mice then received CD19 antigen (as 1e9 pfu Ad-CMV-mCD19t-P2A-eGFP) intratumorally every other day from day 5 to day 11.

FIG. 9 provides data illustrating CD45.1 expression by flow cytometry on day 11 post CAR-T infusion for tumor samples.

FIG. 10 provides data illustrating CD45.1 expression by flow cytometry on day 11 post CAR-T infusion for tumor samples. Null refers to PBS control.

FIG. 11 provides data illustrating PD-1 expression on CD8+CD45.1+ cells by flow cytometry on day 11 post CAR-T infusion for tumor samples.

FIG. 12 provides a graph plotting tumor size versus Days after CAR-T infusion.

FIGS. 13A-13B provide data illustrating CD45.1 expression (FIG. 13A) and PD-1 expression (FIG. 13B) on CD8+CD45.1+ cells by flow cytometry on day 11 post CAR-T infusion for tumor samples.

FIGS. 14A-14D provide data related to the finding that dual targeted CAR T cells display increased tumor burden control and overall survival in “hot” PDA tumors in vivo. FIG. 14A is an experimental design schematic. mCAR T cells were injected 7 days after “hot” PDA tumor engraftment. The mice were intermittently treated with adenovirus (AAV) encoding truncated CD19. FIG. 14B is a graph showing tumor burden (n=10/group, N=2). P values were determined using repeat measures two-way ANOVA with Tukey's multiple comparison test. *p<0.05, **p<0.01, ***p<0.001, ****p<0.0001 compared with A03. +p<0.05, ++p<0.01, +++p<0.001, ++++p<0.0001 compared with A03-AAV. FIG. 14C is a graph showing overall survival for mice that were not administered AAV. P values were determined by Log rank (Mantel-Cox) test. FIG. 14D is a graph showing overall survival for mice that were administered AAV. P values were determined by Log rank (Mantel-Cox) test.

FIGS. 15A-15B provide data related to the finding that dual targeted CAR T cells have increased proliferation and effector phenotype in a “cold” PDA model and induce B cell aplasia in the spleen. The experimental scheme shown in FIG. 14A was adapted for use with a “cold” tumor PDA model. CD45.1+ CAR T cells from the spleen were characterized 6 days after CAR T infusion via bulk RNA-seq (n=5/replicate). FIG. 15A is a plot top differentially expressed genes from the data that were normalized and analyzed using DESeq2. FIG. 15B is a chart showing gene set enrichment analysis prepared using fGSEA package.

### DETAILED DESCRIPTION

Although CAR-T cell therapy has been successful for treating hematological malignancies, it has yet to be effective for treating solid tumors. There are key differences in treating liquid vs. solid tumors. In comparison to hematological malignancies, intravenously administered CAR T cells must traffic throughout the body to find solid tumor sites. T cell trafficking is limited in solid tumors, and also in the case of brain tumors, by the blood-brain barrier. Additionally, tumor heterogeneity is a fundamental property of cancer. As such, targeting one single antigen (Ag) will result in the relapse associated with the loss of that Ag induced by CAR T cell therapy. Meanwhile, CAR-T cells can be rendered dysfunctional by both intrinsic and extrinsic factors. Intrinsic dysfunction primarily results from T cell exhaustion driven by high antigen loads and/or tonic signaling. CAR T cell function can also be suppressed by extrinsic factors, such as cytokines (for example, TGF-β), ligands that signal via inhibitory receptors (for example, PD-L1) or competition for nutrients within the tumor microenvironment (TME). Thus, poor engraftment and limited expansion of CAR T cells are two main causes of the failure of CAR T therapy to eliminate solid tumors.

Furthermore, typical therapeutic doses of CAR T cells in human patients range from about 10⁷ to 10⁹ cells, whereas typical therapeutic doses of CAR T cells used in preclinical mouse models range from 10⁶ to 10⁷ cells, which would be equivalent to 10¹⁰ to 10¹¹ cells in a human patient. But merely increasing therapeutic dose of CAR T cells administered to a human patient is known to increase the risk of on-target-off-tumor toxicity. For example, a recent clinical trial treated mesothelin (MSLN)-positive ovarian and pancreatic cancer patients with T cells expressing a CAR comprising an anti-MSLN scFv (“M5”). The MSLN expression was high in the patient's tumors. However, MSLN was also expressed at lower levels in some normal tissues, such as type 2 pneumocytes in the lung. The results from this trial showed that low dose (3×10⁷) M5 CAR-T cells was well-tolerated by patients but did not provide anti-tumor effects, whereas higher doses (1-3×10⁸) M5 CAR-T cells resulted in severe respiratory problems (potentially due to on-target, off-tumor toxicity) and still had no therapeutic effect. Although local administration of CAR-T cells at a tumor site is possible, this strategy limits the therapeutic potential for metastatic cancers. Overall, this trial showed that low doses of CAR-T cells typically can not eliminate cancers, while higher doses of CAR-T cells cause on-target-off-tumor toxicity with no therapeutic effects either. Therefore, a need exists for strategies to deliver therapeutically-effective doses of CAR-T cells to solid tumors while reducing toxicities.

The present invention comprises the use of CD19 antigen-driven expansion of CAR-expressing immune cells (e.g., CAR T cells) in the peripheral blood (PB) of a patient which safely increases the therapeutic index of adoptive cell therapy targeting solid tumors. This is achieved using a dual CAR approach, wherein a subject receives an immune cell (i.e., a plurality of immune cells, e.g. a plurality of T cells) engineered to express a first CAR targeting CD19 antigen and a second CAR targeting a tumor antigen other than CD19 (i.e., dual CAR T cells; dual CAR immune cells). The CD19 antigen in the subject drives dual CAR T cell expansion. CD19 antigen is present in the subject endogenously, e.g., on CD19-expressing B cells, and/or is provided exogenously, e.g., as CD19 antigen protein, as a cell expressing CD19 antigen, or as a nucleic acid comprising a nucleotide sequence encoding CD19 antigen. Compared to cells expressing a single CAR targeting a tumor antigen, the dual CAR T cell approach of the present invention surprisingly enhances CAR T cell expansion in vivo in both peripheral blood and in organs of the subject, enhances and accelerates tumor cell killing in vitro and in vivo (e.g., solid tumor cell killing and tumor size reduction), increases cytotoxic cytokine expression, and lowers on-target off-tumor cytotoxicity. Importantly, boosting and anti-tumor efficacy does not require lymphodepletion. Additionally, the boosted dual CAR T cells of the present invention are primarily an effector memory or a central memory phenotype and express only one or two exhaustion markers. Importantly, these unexpected effects are not mere additive effects of combining two CARs and the present invention addresses a long unmet need for treating solid tumors with adoptive cell therapy.

In one aspect, the invention provides an isolated nucleic acid comprising: (a) a first nucleotide sequence encoding a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second nucleotide sequence encoding a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In another aspect, the invention provides a modified cell, wherein the cell is an immune cell or precursor cell thereof, and wherein the cell is engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In another aspect, the invention provides a method of treating cancer in a subject in need thereof, comprising administering to the subject a population of modified cells, wherein the cells are immune cells or precursor cells thereof, and wherein the cells are engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In other aspects, provided herein are related vectors, compositions (e.g., pharmaceutical compositions), and kits.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Methods and techniques using T Cells with chimeric antigen receptors (CAR T cells) are described in e.g., Ruella, et al., J. Clin. Invest., 126(10):3814-3826 (2016) and Kalos, et al., 3 (95), 95ra73:1-11 (2011), the contents of which are hereby incorporated by reference in their entireties.

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is a human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Chimeric Antigen Receptors

The present invention provides a modified immune cell or precursor cell thereof (e.g., a modified T cell) engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain. Nucleic acids comprising nucleotide sequences encoding the first and second CAR, vectors comprising the nucleic acids, and modified cells (e.g. modified T cells) comprising the first and second CAR, the vector, and/or the nucleic acid, are also provided. The antigen binding domain of the first or second CAR is operably linked to another domain of the CAR, such as a hinge, a transmembrane domain or an intracellular domain, each described elsewhere herein, for expression in the cell. In one embodiment, a first nucleotide sequence encoding the antigen binding domain is operably linked to a second nucleotide sequence encoding a hinge and/or transmembrane domain, and further operably linked to a third nucleotide sequence encoding an intracellular domain.

The antigen binding domain described herein can be combined with any of the transmembrane domains described herein, any of the intracellular domains or cytoplasmic domains described herein, or any of the other domains described herein that may be included in a CAR of the present invention, such as a hinge domain or a spacer sequence. The CAR of the present invention may also include a leader sequence. The CAR of the present invention may also include one or more spacer domains or linkers as described herein which may serve to link one domain of the CAR to the next domain.

### Antigen Binding Domain

The extracellular antigen binding domain of a CAR serves to recognize (i.e., bind to) a specific target antigen, which antigens may include proteins, carbohydrates, and glycolipids. The first CAR of the invention comprises an extracellular antigen binding domain having affinity for CD19. The first CAR further comprises a transmembrane domain and an intracellular domain comprising at least one costimulatory domain and a signaling domain as described elsewhere herein. In some embodiments, the first CAR comprises a CD28 costimulatory domain and a CD3 zeta signaling domain.

CD19 is naturally expressed on B cells and was selected as the antigen to be recognized by the first CAR for “boosting” CAR T cell expansion in the peripheral blood because (i) the adoptively transferred cells will encounter CD19 immediately upon entering the patient's circulation, (ii) there is a high amount of natural CD19 in the body (Morbach, et al., Clinical and Experimental Immunology, 2010), and (iii) CD19 can be self-regenerated from hematopoietic stem cells (HSCs) which differentiate into B cells. This can potentially sustain the persistent existence of CAR-T cells in patients as long as the patient has healthy HSCs. Additionally, treatment with replacement immunoglobulin infusions can restore a patient's immunoglobulin levels to normal, even if the patient experiences B cell aplasia after receiving anti-CD19 (e.g., CTL019) CAR-T cells. Further, large-scale real world analysis does not reveal a significant risk of infection in patients treated with CTL019 (Schultz, et al., J. Clin. Oncol., 2021:JCO. 20.03585). Anti-CD19 antigen binding domains are known in the art. For example, the anti-CD19 “FMC63” scFv. The first CAR further comprises a transmembrane domain and an intracellular domain comprising at least one costimulatory domain and a signaling domain. In some embodiments, the first CAR comprises a CD28 costimulatory domain and a CD3 zeta signaling domain.

The second CAR of the invention comprises an extracellular antigen binding domain having affinity for a tumor antigen, wherein the tumor antigen is not CD19. The second CAR further comprises a transmembrane domain, and an intracellular domain comprising at least one costimulatory domain and a signaling domain as described elsewhere herein. In some embodiments, the second CAR comprises a 4-1BB costimulatory domain. In some embodiments, the second CAR comprises a CD3 zeta signaling domain. In some embodiments, the second CAR comprises an intracellular domain comprising a 4-1BB costimulatory domain and a CD3 zeta signaling domain. The second CAR of the invention targets any tumor antigen other than CD19, examples of which include, but are not limited to, CARs targeting TnMuc1 (see, e.g., WO2020198413A1), GFRα4 (see, WO2016025880A1), PSMA (see, e.g., WO2020181094A1), EGFR (see, e.g., WO2021041725A1 and WO2020210768A1), IL13Rα2 (see, e.g., WO2021041725A1 and WO2020210768A1), and mesothelin (see, e.g., WO2017/112741).

Suitable tumor antigens which the second CAR targets (i.e., binds to) are known in the art and include, but are not limited to, alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), Glycosyl-phosphatidylinositol (GPI)-linked GDNF family α-receptor 4 (GFRα4; GFRalpha4), HER2, HLA-A2, ICAM1, interleukin 13 receptor subunit alpha (IL3Rα), interleukin 13 receptor subunit alpha 1 (IL13Rα1), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), VEGFR2, and any combination thereof. In some embodiments, the tumor antigen is selected from prostate specific membrane antigen (PSMA), MUC1, Tn-glycoform of MUC1 (TnMUC1), Glycosyl-phosphatidylinositol (GPI)-linked GDNF family α-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

In one embodiment, the modified immune cell or precursor cell thereof (e.g., a modified T cell) is engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for at least one tumor antigen, (ii) a second transmembrane domain, and (iii) a second intracellular domain comprising at least one costimulatory domain and a signaling domain; wherein the at least one tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), Glycosyl-phosphatidylinositol (GPI)-linked GDNF family α-receptor 4 (GFRα4; GFRalpha4), HER2, HLA-A2, ICAM1, interleukin 13 receptor subunit alpha (IL3Rα), interleukin 13 receptor subunit alpha 1 (IL13Rα1), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), VEGFR2, and any combination thereof. In some embodiments, the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family α-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

In one embodiment, the modified immune cell or precursor cell thereof (e.g., a modified T cell) is engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, (ii) a second transmembrane domain, and (iii) a second intracellular domain comprising at least one costimulatory domain and a signaling domain; wherein the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family α-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

The antigen binding domain of a CAR can include any domain that binds to the antigen and may include, but is not limited to, a monoclonal antibody (mAb), a polyclonal antibody, a synthetic antibody, a human antibody, a humanized antibody, a non-human antibody, a single-domain antibody, a full length antibody or any antigen-binding fragment thereof, a Fab, and a single-chain variable fragment (scFv). In some embodiments, the antigen binding domain comprises an aglycosylated antibody or a fragment thereof or scFv thereof.

As used herein, the term “single-chain variable fragment” or “scFv” is a fusion protein of the variable regions of the heavy (VH) and light (VL) chains of an immunoglobulin (e.g., mouse or human) covalently linked to form a VH::VL heterodimer. The variable heavy (VH) and light (VL) chains are either joined directly or joined by a peptide linker, which connects the N-terminus of the VH with the C-terminus of the VL, or the C-terminus of the VH with the N-terminus of the VL. In some embodiments, the antigen binding domain (e.g., tumor antigen binding domain) comprises an scFv having the configuration from N-terminus to C-terminus, VH-linker-VL. In some embodiments, the antigen binding domain comprises an scFv having the configuration from N-terminus to C-terminus, VL-linker-VH or VH-linker-VL. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers. Those of skill in the art would be able to select the appropriate linker sequence for use in the present invention. In one embodiment, an antigen binding domain of the present invention comprises a heavy chain variable region (VH) and a light chain variable region (VL), wherein the VH and VL are separated by a linker sequence.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In other embodiments, the antigen binding domain comprises an antibody mimetic protein such as, for example, designed ankyrin repeat protein (DARPin), affibody, monobody, (i.e., adnectin), affilin, affimer, affitin, alphabody, avimer, Kunitz domain peptide, or anticalin. Constructs with specific binding affinities can be generated using DARPin libraries e.g., as described in Seeger, et al., Protein Sci., 22:1239-1257 (2013).

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof, or a humanized murine antibody or a fragment thereof.

In certain embodiments, the antigen binding domain comprises a heavy chain variable region that comprises three heavy chain complementarity determining regions (HCDRs) and a light chain variable region that comprises three light chain complementarity determining regions (LCDRs). In certain embodiments, the antigen binding domain comprises a linker.

### Transmembrane Domain

CARs of the present invention comprise a transmembrane domain that connects the antigen binding domain of the CAR to the intracellular domain of the CAR. The transmembrane domain of the CAR is a region that is capable of spanning the plasma membrane of a cell (e.g., an immune cell or precursor thereof). In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), ICOS, CD278, Toll-like receptor 1 (TLR1), TLR2, TLR3, TLR4, TLR5, TLR6, TLR7, TLR8, TLR9 or a transmembrane domain derived from a killer immunoglobulin-like receptor (KIR).

In certain embodiments, the transmembrane domain comprises a transmembrane domain of CD8. In certain embodiments, the transmembrane domain of CD8 is a transmembrane domain of CD8α.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in the CAR, such as a hinge domain or region.

In some embodiments, the transmembrane domain further comprises a hinge region. The CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, the CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). The hinge region can comprise an amino acid sequence of a human IgG1, IgG2, IgG3, or IgG4, hinge region (see, e.g., Yan et al., J. Biol. Chem. (2012) 287: 5891-5897). In one embodiment, the hinge region can comprise an amino acid sequence derived from human CD8, or a variant thereof.

### Intracellular Signaling Domain

The CAR of the present invention also includes an intracellular signaling domain. The terms “intracellular signaling domain,” “signaling domain,” “intracellular domain,” and “ICD” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the immune cell (i.e., the T cell), as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may comprise an intracellular signaling domain (i.e., “ICD”) of a protein selected from human CD3 zeta chain, FcyRIII, FcsRI, DAP10, DAP12, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, such as any synthetic sequence thereof, that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGBl, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in the CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of the CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire molecule. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in the CAR.

In certain embodiments, the intracellular domain comprises a costimulatory domain of 4-1BB. In certain embodiments, the intracellular domain comprises an intracellular domain of CD3ζ or a variant thereof. In certain embodiments, the intracellular domain comprises a costimulatory domain of 4-1BB and an intracellular domain of CD3ζ.

Tolerable variations of the individual CAR domain sequences (leader, antigen binding domain, hinge, transmembrane, and/or intracellular domains) will be known to those of skill in the art. For example, in certain embodiments the CAR domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known sequence.

Exemplary amino acid and nucleotide sequences for the first and second CARs disclosed herein are as follows:

Murine anti-CD19 CAR AA sequence
(SEQ ID NO: 1)
MASPLTRFLSLNLLLLGESIILGSGEADIQMTQSPASLSTSLGETVTIQC
QASEDIYSGLAWYQQKPGKSPQLLIYGASDLQDGVPSRFSGSGSGTQYSL
KITSMQTEDEGVYFCQQGLTYPRTFGGGTKLELKGGGGSGGGGSGGGGSE
VQLQQSGAELVRPGTSVKLSCKVSGDTITFYYMHFVKQRPGQGLEWIGRI
DPEDESTKYSEKFKNKATLTADTSSNTAYLKLSSLTSEDTATYFCIYGGY
YFDYWGQGVMVTVSSLQKVNSTTTKPVLRTPSPVHPTGTSQPQRPEDCRP
RGSVKGTGLDFACDIYFWALVVVAGVLFCYGLLVTVALCVIWTNSRRNRL
LQSDYMNMTPRRPGLTRKPYQPYAPARDFAAYRPKFSRSAETAANLQDPN
QLYNELNLGRREEYDVLEKKRARDPEMGGKQQRRRNPQEGVYNALQKDKM
AEAYSEIGTKGERRRGKGHDGLYQGLSTATKDTYDALHMQTLAPR
Murine anti-CD19 CAR NT sequence
(SEQ ID NO: 2)
ATGGCCTCACCCCTGACCAGATTCCTGTCACTGAACCTGCTGCTGCTGGG
CGAGTCAATCATCCTGGGCTCAGGCGAGGCCGACATCCAGATGACCCAGA
GCCCTGCCAGCCTGTCTACCAGCCTGGGCGAGACAGTGACCATCCAGTGT
CAGGCCAGCGAGGACATCTACTCTGGCCTGGCTTGGTATCAGCAGAAGCC
CGGCAAGAGCCCTCAGCTGCTGATCTACGGCGCCAGCGACCTGCAGGACG
GCGTGCCTAGCAGATTCAGCGGCAGCGGCTCCGGAACCCAGTACAGCCTG
AAGATCACCAGCATGCAGACCGAGGACGAGGGCGTGTACTTCTGCCAGCA
AGGCCTGACCTACCCTAGAACCTTCGGAGGAGGCACCAAGCTGGAACTGA
AGGGCGGAGGCGGAAGTGGAGGCGGAGGATCTGGCGGCGGAGGCTCTGAA
GTGCAGCTGCAGCAGTCTGGCGCTGAACTGGTCCGGCCTGGCACTAGCGT
GAAGCTGTCCTGCAAGGTGTCCGGCGACACCATCACCTTCTACTACATGC
ACTTCGTGAAGCAGAGGCCAGGACAGGGCCTGGAATGGATCGGCAGAATC
GACCCTGAGGACGAGAGCACCAAGTACAGCGAGAAGTTCAAGAACAAGGC
CACCCTGACCGCCGACACCAGCAGCAACACCGCCTACCTGAAGCTGTCTA
GCCTGACCTCCGAGGACACCGCCACCTACTTTTGCATCTACGGCGGCTAC
TACTTCGACTACTGGGGCCAGGGCGTGATGGTCACCGTGTCCAGCCTGCA
GAAGGTGAACTCAACCACCACCAAGCCCGTGCTGAGAACCCCCTCACCCG
TGCACCCCACCGGCACCTCACAGCCCCAGAGACCCGAGGACTGCAGACCC
AGAGGCTCAGTGAAGGGCACCGGCCTGGACTTCGCCTGCGACATCTACTT
TTGGGCACTGGTCGTGGTTGCTGGAGTCCTGTTTTGTTATGGCTTGCTAG
TGACAGTGGCTCTTTGTGTTATCTGGACAAATAGTAGAAGGAACAGACTC
CTTCAAAGTGACTACATGAACATGACTCCCCGGAGGCCTGGGCTCACTCG
AAAGCCTTACCAGCCCTACGCCCCTGCCAGAGACTTTGCAGCGTACCGCC
CCAAGTTCTCAAGATCAGCCGAGACCGCCGCCAACCTGCAGGACCCCAAC
CAGCTGTACAACGAGCTGAACCTGGGCAGAAGAGAGGAGTACGACGTGCT
GGAGAAGAAGAGAGCCAGAGACCCCGAGATGGGCGGCAAGCAGCAGAGAA
GAAGAAACCCCCAGGAGGGCGTGTACAACGCCCTGCAGAAGGACAAGATG
GCCGAGGCCTACTCAGAGATCGGCACCAAGGGCGAGAGAAGAAGAGGCAA
GGGCCACGACGGCCTGTACCAGGGCCTGTCAACCGCCACCAAGGACACCT
ACGACGCCCTGCACATGCAGACCCTGGCCCCCAGA
Human anti-CD19 CAR AA sequence
(SEQ ID NO: 3)
MALPVTALLLPLALLLHAARPEIVMTQSPATLSLSPGERATLSCRASQDI
SKYLNWYQQKPGQAPRLLIYHTSRLHSGIPARFSGSGSGTDYTLTISSLQ
PEDFAVYFCQQGNTLPYTFGQGTKLEIKGGGGSGGGGSGGGGSQVQLQES
GPGLVKPSETLSLTCTVSGVSLPDYGVSWIRQPPGKGLEWIGVIWGSETT
YYQSSLKSRVTISKDNSKNQVSLKLSSVTAADTAVYYCAKHYYYGGSYAM
DYWGQGTLVTVSSTTTPAPRPPTPAPTIASQPLSLRPEACRPAAGGAVHT
RGLDFACDFWVLVVVGGVLACYSLLVTVAFIIFWVRSKRSRLLHSDYMNM
TPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR
Human anti-CD19 CAR NT sequence
(SEQ ID NO: 4)
ATGGCCTTACCAGTGACCGCCTTGCTCCTGCCGCTGGCCTTGCTGCTCCA
CGCCGCCAGGCCGGAAATTGTGATGACCCAGTCACCCGCCACTCTTAGCC
TTTCACCCGGTGAGCGCGCAACCCTGTCTTGCAGAGCCTCCCAAGACATC
TCAAAATACCTTAATTGGTATCAACAGAAGCCCGGACAGGCTCCTCGCCT
TCTGATCTACCACACCAGCCGGCTCCATTCTGGAATCCCTGCCAGGTTCA
GCGGTAGCGGATCTGGGACCGACTACACCCTCACTATCAGCTCACTGCAG
CCAGAGGACTTCGCTGTCTATTTCTGTCAGCAAGGGAACACCCTGCCCTA
CACCTTTGGACAGGGCACCAAGCTCGAGATTAAAGGTGGAGGTGGCAGCG
GAGGAGGTGGGTCCGGCGGTGGAGGAAGCCAGGTCCAACTCCAAGAAAGC
GGACCGGGTCTTGTGAAGCCATCAGAAACTCTTTCACTGACTTGTACTGT
GAGCGGAGTGTCTCTCCCCGATTACGGGGTGTCTTGGATCAGACAGCCAC
CGGGGAAGGGTCTGGAATGGATTGGAGTGATTTGGGGCTCTGAGACTACT
TACTACCAATCATCCCTCAAGTCACGCGTCACCATCTCAAAGGACAACTC
TAAGAATCAGGTGTCACTGAAACTGTCATCTGTGACCGCAGCCGACACCG
CCGTGTACTATTGCGCTAAGCATTACTATTATGGCGGGAGCTACGCAATG
GATTACTGGGGACAGGGTACTCTGGTCACCGTGTCCAGCACCACGACGCC
AGCGCCGCGACCACCAACACCGGCGCCCACCATCGCGTCGCAGCCCCTGT
CCCTGCGCCCAGAGGCGTGCCGGCCAGCGGCGGGGGGCGCAGTGCACACG
AGGGGGCTGGACTTCGCCTGTGATTTTTGGGTGCTGGTGGTGGTTGGTGG
AGTCCTGGCTTGCTATAGCTTGCTAGTAACAGTGGCCTTTATTATTTTCT
GGGTGAGGAGTAAGAGGAGCAGGCTCCTGCACAGTGACTACATGAACATG
ACTCCCCGCCGCCCCGGGCCCACCCGCAAGCATTACCAGCCCTATGCCCC
ACCACGCGACTTCGCAGCCTATCGCTCCAGAGTGAAGTTCAGCAGGAGCG
CAGACGCCCCCGCGTACCAGCAGGGCCAGAACCAGCTCTATAACGAGCTC
AATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCC
TGTACAATGAACTGCAGAAAGATAAGATGGCGGAGGCCTACAGTGAGATT
GGGATGAAAGGCGAGCGCCGGAGGGGCAAGGGGCACGATGGCCTTTACCA
GGGTCTCAGTACAGCCACCAAGGACACCTACGACGCCCTTCACATGCAGG
CCCTGCCCCCTCGC
Murine anti-MSLN CAR AA sequence
(SEQ ID NO: 5)
MASPLTRFLSLNLLLLGESIILGSGEAAQVQLQESGPGLVKPSQTLSLTC
TVSGGSISSGGYYWSWIRQHPGKGLEWIGYIYYSGSTYYNPSLKSRVTIS
VDTSKNQFSLKLSSVTAADTAVYYCARFDYGDFYDAFDIWGQGTMVTVSS
GGGGSGGGGSGGGGSEIVLTQSPSSLSASVGDRVTITCRASQGISSALAW
YQQKPGKAPKLLIYDASSLESGVPSRFSGSGSGTDFTLTISSLQPEDFAT
YYCQQFNSYPITFGQGTRLEIKRLQKVNSTTTKPVLRTPSPVHPTGTSQP
QRPEDCRPRGSVKGTGLDFACDIYIWAPLAGICVALLLSLIITLIKWIRK
KFPHIFKQPFKKTTGAAQEEDACSCRCPQEEEGGGGGYELKFSRSAETAA
NLQDPNQLYNELNLGRREEYDVLEKKRARDPEMGGKQQRRRNPQEGVYNA
LQKDKMAEAYSEIGTKGERRRGKGHDGLYQGLSTATKDTYDALHMQTLAP
R
Murine anti-MSLN CAR NT sequence
(SEQ ID NO: 6)
ATGGCCAGCCCCCTGACCAGATTCCTGAGCCTGAACCTGCTGCTGCTGGG
CGAGAGCATCATCCTGGGCAGCGGCGAGGCCGCCCAGGTGCAGCTGCAGG
AGTCGGGCCCAGGACTGGTGAAGCCTTCACAGACCCTGTCCCTCACCTGC
ACTGTCTCTGGTGGCTCCATCAGCAGTGGTGGTTACTACTGGAGCTGGAT
CCGCCAGCACCCAGGGAAGGGCCTGGAGTGGATTGGGTACATCTATTACA
GTGGGAGCACCTACTACAACCCGTCCCTCAAGAGTCGAGTTACCATATCA
GTAGACACGTCCAAGAACCAGTTCTCCCTGAAGCTGAGCTCTGTGACCGC
TGCGGACACGGCCGTGTATTACTGTGCGAGATTTGACTACGGTGACTTCT
ATGATGCTTTTGATATCTGGGGCCAAGGGACAATGGTCACCGTCTCTTCA
GGTGGTGGTGGTAGCGGCGGCGGCGGCTCTGGTGGTGGTGGATCCGAAAT
TGTGTTGACGCAGTCTCCATCCTCCCTGTCTGCATCTGTAGGAGACAGAG
TCACCATCACTTGCCGGGCAAGTCAGGGCATTAGCAGTGCTTTAGCCTGG
TATCAGCAGAAACCAGGGAAAGCTCCTAAGCTCCTGATCTATGATGCCTC
CAGTTTGGAAAGTGGGGTCCCATCAAGGTTCAGCGGCAGTGGATCTGGGA
CAGATTTCACTCTCACCATCAGCAGCCTGCAGCCTGAAGATTTTGCAACT
TATTACTGTCAACAGTTTAATAGTTACCCGATCACCTTCGGCCAAGGGAC
ACGACTGGAGATTAAACGTCTGCAGAAGGTGAACAGCACCACCACCAAGC
CCGTGCTGAGAACCCCCAGCCCCGTGCACCCCACCGGCACCAGCCAGCCC
CAGAGACCCGAGGACTGCAGACCCAGAGGCAGCGTGAAGGGCACCGGCCT
GGACTTCGCCTGCGACATCTACATCTGGGCCCCCCTGGCCGGCATCTGCG
TGGCCCTGCTGCTGAGCCTGATCATCACCCTGATCAAGTGGATCAGAAAG
AAGTTCCCCCACATCTTCAAGCAGCCCTTCAAGAAGACCACCGGCGCCGC
CCAGGAGGAGGACGCCTGCAGCTGCAGATGCCCCCAGGAGGAGGAGGGCG
GCGGCGGCGGCTACGAGCTGAAGTTCAGCAGAAGCGCCGAGACCGCCGCC
AACCTGCAGGACCCCAACCAGCTGTACAACGAGCTGAACCTGGGCAGAAG
AGAGGAGTACGACGTGCTGGAGAAGAAGAGAGCCAGAGACCCCGAGATGG
GCGGCAAGCAGCAGAGAAGAAGAAACCCCCAGGAGGGCGTGTACAACGCC
CTGCAGAAGGACAAGATGGCCGAGGCCTACAGCGAGATCGGCACCAAGGG
CGAGAGAAGAAGAGGCAAGGGCCACGACGGCCTGTACCAGGGCCTGAGCA
CCGCCACCAAGGACACCTACGACGCCCTGCACATGCAGACCCTGGCCCCC
AGA
Human anti-MSLN CAR AA sequence
(SEQ ID NO: 7)
MALPVTALLLPLALLLHAARPQVQLVQSGAEVEKPGASVKVSCKASGYTF
TDYYMHWVRQAPGQGLEWMGWINPNSGGTNYAQKFQGRVTMTRDTSISTA
YMELSRLRSDDTAVYYCASGWDFDYWGQGTLVTVSSGGGGSGGGGSGGGG
SGGGGSDIVMTQSPSSLSASVGDRVTITCRASQSIRYYLSWYQQKPGKAP
KLLIYTASILQNGVPSRFSGSGSGTDFTLTISSLQPEDFATYYCLQTYTT
PDFGPGTKVEIKTTTPAPRPPTPAPTIASQPLSLRPEACRPAAGGAVHTR
GLDFACDIYIWAPLAGTCGVLLLSLVITLYCKRGRKKLLYIFKQPFMRPV
QTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR
Human anti-MSLN CAR NT sequence
(SEQ ID NO: 8)
ATGGCCCTCCCTGTCACCGCCCTGCTGCTTCCGCTGGCTCTTCTGCTCCA
CGCCGCTCGGCCCCAAGTCCAACTCGTTCAATCAGGCGCAGAAGTCGAAA
AGCCCGGAGCATCAGTCAAAGTCTCTTGCAAGGCTTCCGGCTACACCTTC
ACGGACTACTACATGCACTGGGTGCGCCAGGCTCCAGGCCAGGGACTGGA
GTGGATGGGATGGATCAACCCGAATTCCGGGGGAACTAACTACGCCCAGA
AGTTTCAGGGCCGGGTGACTATGACTCGCGATACCTCGATCTCGACTGCG
TACATGGAGCTCAGCCGCCTCCGGTCGGACGATACCGCCGTGTACTATTG
TGCGTCGGGATGGGACTTCGACTACTGGGGGCAGGGCACTCTGGTCACTG
TGTCAAGCGGAGGAGGTGGATCAGGTGGAGGTGGAAGCGGGGGAGGAGGT
TCCGGCGGCGGAGGATCAGATATCGTGATGACGCAATCGCCTTCCTCGTT
GTCCGCATCCGTGGGAGACAGGGTGACCATTACTTGCAGAGCGTCCCAGT
CCATTCGGTACTACCTGTCGTGGTACCAGCAGAAGCCGGGGAAAGCCCCA
AAACTGCTTATCTATACTGCCTCGATCCTCCAAAACGGCGTGCCATCAAG
ATTCAGCGGTTCGGGCAGCGGGACCGACTTTACCCTGACTATCAGCAGCC
TGCAGCCGGAAGATTTCGCCACGTACTACTGCCTGCAAACCTACACCACC
CCGGACTTCGGACCTGGAACCAAGGTGGAGATCAAGACCACTACCCCAGC
ACCGAGGCCACCCACCCCGGCTCCTACCATCGCCTCCCAGCCTCTGTCCC
TGCGTCCGGAGGCATGTAGACCCGCAGCTGGTGGGGCCGTGCATACCCGG
GGTCTTGACTTCGCCTGCGATATCTACATTTGGGCCCCTCTGGCTGGTAC
TTGCGGGGTCCTGCTGCTTTCACTCGTGATCACTCTTTACTGTAAGCGCG
GTCGGAAGAAGCTGCTGTACATCTTTAAGCAACCCTTCATGAGGCCTGTG
CAGACTACTCAAGAGGAGGACGGCTGTTCATGCCGGTTCCCAGAGGAGGA
GGAAGGCGGCTGCGAACTGCGCGTGAAATTCAGCCGCAGCGCAGATGCTC
CAGCCTACAAGCAGGGGCAGAACCAGCTCTACAACGAACTCAATCTTGGT
CGGAGAGAGGAGTACGACGTGCTGGACAAGCGGAGAGGACGGGACCCAGA
AATGGGCGGGAAGCCGCGCAGAAAGAATCCCCAAGAGGGCCTGTACAACG
AGCTCCAAAAGGATAAGATGGCAGAAGCCTATAGCGAGATTGGTATGAAA
GGGGAACGCAGAAGAGGCAAAGGCCACGACGGACTGTACCAGGGACTCAG
CACCGCCACCAAGGACACCTATGACGCTCTTCACATGCAGGCCCTGCCGC
CTCGG
A03 Murine anti-MSLN scFv nucleotide sequence
(SEQ ID NO: 9)
GCCCAGGTGCAGCTGCAGGAGTCGGGCCCAGGACTGGTGAAGCCTTCACA
GACCCTGTCCCTCACCTGCACTGTCTCTGGTGGCTCCATCAGCAGTGGTG
GTTACTACTGGAGCTGGATCCGCCAGCACCCAGGGAAGGGCCTGGAGTGG
ATTGGGTACATCTATTACAGTGGGAGCACCTACTACAACCCGTCCCTCAA
GAGTCGAGTTACCATATCAGTAGACACGTCCAAGAACCAGTTCTCCCTGA
AGCTGAGCTCTGTGACCGCTGCGGACACGGCCGTGTATTACTGTGCGAGA
TTTGACTACGGTGACTTCTATGATGCTTTTGATATCTGGGGCCAAGGGAC
AATGGTCACCGTCTCTTCAGGTGGTGGTGGTAGCGGCGGCGGCGGCTCTG
GTGGTGGTGGATCCGAAATTGTGTTGACGCAGTCTCCATCCTCCCTGTCT
GCATCTGTAGGAGACAGAGTCACCATCACTTGCCGGGCAAGTCAGGGCAT
TAGCAGTGCTTTAGCCTGGTATCAGCAGAAACCAGGGAAAGCTCCTAAGC
TCCTGATCTATGATGCCTCCAGTTTGGAAAGTGGGGTCCCATCAAGGTTC
AGCGGCAGTGGATCTGGGACAGATTTCACTCTCACCATCAGCAGCCTGCA
GCCTGAAGATTTTGCAACTTATTACTGTCAACAGTTTAATAGTTACCCGA
TCACCTTCGGCCAAGGGACACGACTGGAGATTAAACGT
A03 Murine anti-MSLN scFv amino acid sequence
(SEQ ID NO: 10)
AQVQLQESGPGLVKPSQTLSLTCTVSGGSISSGGYYWSWIRQHPGKGLEW
IGYIYYSGSTYYNPSLKSRVTISVDTSKNQFSLKLSSVTAADTAVYYCAR
FDYGDFYDAFDIWGQGTMVTVSSGGGGSGGGGSGGGGSEIVLTQSPSSLS
ASVGDRVTITCRASQGISSALAWYQQKPGKAPKLLIYDASSLESGVPSRF
SGSGSGTDFTLTISSLQPEDFATYYCQQFNSYPITFGQGTRLEIKR
M5 Human anti-MSLN scFv nucleotide sequence
(SEQ ID NO: 11)
CAAGTCCAACTCGTTCAATCAGGCGCAGAAGTCGAAAAGCCCGGAGCATC
AGTCAAAGTCTCTTGCAAGGCTTCCGGCTACACCTTCACGGACTACTACA
TGCACTGGGTGCGCCAGGCTCCAGGCCAGGGACTGGAGTGGATGGGATGG
ATCAACCCGAATTCCGGGGGAACTAACTACGCCCAGAAGTTTCAGGGCCG
GGTGACTATGACTCGCGATACCTCGATCTCGACTGCGTACATGGAGCTCA
GCCGCCTCCGGTCGGACGATACCGCCGTGTACTATTGTGCGTCGGGATGG
GACTTCGACTACTGGGGGCAGGGCACTCTGGTCACTGTGTCAAGCGGAGG
AGGTGGATCAGGTGGAGGTGGAAGCGGGGGAGGAGGTTCCGGCGGCGGAG
GATCAGATATCGTGATGACGCAATCGCCTTCCTCGTTGTCCGCATCCGTG
GGAGACAGGGTGACCATTACTTGCAGAGCGTCCCAGTCCATTCGGTACTA
CCTGTCGTGGTACCAGCAGAAGCCGGGGAAAGCCCCAAAACTGCTTATCT
ATACTGCCTCGATCCTCCAAAACGGCGTGCCATCAAGATTCAGCGGTTCG
GGCAGCGGGACCGACTTTACCCTGACTATCAGCAGCCTGCAGCCGGAAGA
TTTCGCCACGTACTACTGCCTGCAAACCTACACCACCCCGGACTTCGGAC
CTGGAACCAAGGTGGAGATCAAG
M5 Human anti-MSLN scFv amino acid sequence
(SEQ ID NO: 12)
QVQLVQSGAEVEKPGASVKVSCKASGYTFTDYYMHWVRQAPGQGLEWMGW
INPNSGGTNYAQKFQGRVTMTRDTSISTAYMELSRLRSDDTAVYYCASGW
DFDYWGQGTLVTVSSGGGGSGGGGSGGGGSGGGGSDIVMTQSPSSLSASV
GDRVTITCRASQSIRYYLSWYQQKPGKAPKLLIYTASILQNGVPSRFSGS
GSGTDFTLTISSLQPEDFATYYCLQTYTTPDFGPGTKVEIK

Anti-MSLN scFv is one example of a second extracellular antigen binding domain having affinity for a tumor antigen, wherein the tumor antigen is not CD19. In some embodiments, the tumor antigen that is not CD19 is mesothelin. In some embodiments, the anti-MSLN scFv comprises an amino acid sequence that is at least about 95%, at least about 96%, at least about 97%, at least about 98%, or at least about 99% identical to the amino acid sequence of the M5 Human anti-MSLN-scFv. In some embodiments, the anti-MSLN scFv is an M11 human anti-MSLN scFv.

Amino acid and nucleotide sequences for additional exemplary second extracellular antigen binding domains having affinity for a tumor antigen include, but are not limited to, the following:

Anti-GD2 scFv nucleotide sequence
(SEQ ID NO: 13)
ATGGCCTTACCAGTGACCGCCTTGCTCCTGCCGCTGGCCTTGCTGCTCCACGCCGCCAGGCC
GGGATCCGATGTTGTGATGACCCAAACTCCACTCTCCCTGCCTGTCAGTCTTGGAGATCAAG
CCTCCATCTCTTGCAGATCTAGTCAGAGTCTTGTACACCGTAACGGAAACACCTATTTACAT
TGGTACCTGCAGAAGCCAGGCCAGTCTCCAAAGCTCCTGATTCACAAAGTTTCCAACCGATT
TTCTGGGGTCCCAGACAGGTTCAGTGGCAGTGGATCAGGGACAGATTTCACACTCAAGATCA
GCAGAGTGGAGGCTGAGGATCTGGGAGTTTATTTCTGTTCTCAAAGTACACACGTTCCTCCG
CTCACGTTCGGTGCTGGGACCAAGCTGGAGCTGAAAGGAGGTGGCGGGTCAGGGGGTGGCGG
AAGCGGAGGCGGCGGTTCAGGCGGAGGAGGCTCGGAGGTGCAGCTTCTGCAGTCTGGACCTG
AGCTGGAGAAGCCTTCCGCTTCAGTGATGATATCCTGCAAGGCTTCTGGTTCCTCCTTCACT
GGCTACAACATGAACTGGGTGAGGCAGAATATTGGAAAGAGCCTTGAATGGATTGGAGCTAT
TGATCCTTACTACGGTGGAACTAGCTACAACCAGAAGTTCAAGGGCAGGGCCACATTGACTG
TAGACAAATCGTCCAGCACAGCCTACATGCACCTCAAGAGCCTGACATCTGAGGACTCTGTC
TATTACTGTGTAAGCGGAATGGAGTACTGGGGTCAAGGAACCTCAGTCACCGTCTCCTCATC
CGGA
Anti-GD2 scFv amino acid sequence
(SEQ ID NO: 14)
MALPVTALLLPLALLLHAARPGSDVVMTQTPLSLPVSLGDQASISCRSSQSLVHRNG
NTYLHWYLQKPGQSPKLLIHKVSNRFSGVPDRESGSGSGTDFTLKISRVEAEDLGVYFCSQS
THVPPLTFGAGTKLELKGGGGSGGGGSGGGGSGGGGSEVQLLQSGPELEKPSASVMISCKAS
GSSFTGYNMNWVRQNIGKSLEWIGAIDPYYGGTSYNQKFKGRATLTVDKSSSTAYMHLKSLT
SEDSVYYCVSGMEYWGQGTSVTVSSSG
Anti-HER2 scFv (high affinity) nucleotide sequence
(SEQ ID NO: 15)
ATGGATTTTCAGGTGCAGATTTTCAGCTTCCTGCTAATCAGTGCCTCAGTCATAATGTCCAG
AGGAGATATCCAGATGACCCAGTCCCCGAGCTCCCTGTCCGCCTCTGTGGGCGATAGGGTCA
CCATCACCTGCCGTGCCAGTCAGGATGTGAATACTGCTGTAGCCTGGTATCAACAGAAACCA
GGAAAAGCTCCGAAACTACTGATTTACTCGGCATCCTTCCTTTATTCTGGAGTCCCTTCTCG
CTTCTCTGGATCTAGATCTGGGACGGATTTCACTCTGACCATCAGCAGTCTGCAGCCGGAAG
ACTTCGCAACTTATTACTGTCAGCAACATTATACTACTCCTCCCACGTTCGGACAGGGTACC
AAGGTGGAGATCAAACGCACTGGGTCTACATCTGGATCTGGGAAGCCGGGTTCTGGTGAGGG
TTCTGAGGTTCAGCTGGTGGAGTCTGGCGGTGGCCTGGTGCAGCCAGGGGGCTCACTCCGTT
TGTCCTGTGCAGCTTCTGGCTTCAACATTAAAGACACCTATATACACTGGGTGCGTCAGGCC
CCGGGTAAGGGCCTGGAATGGGTTGCAAGGATTTATCCTACGAATGGTTATACTAGATATGC
CGATAGCGTCAAGGGCCGTTTCACTATAAGCGCAGACACATCCAAAAACACAGCCTACCTGC
AGATGAACAGCCTGCGTGCTGAGGACACTGCCGTCTATTATTGTTCTAGATGGGGAGGGGAC
GGCTTCTATGCTATGGACGTGTGGGGTCAAGGAACCCTGGTCACCGTCTCCTCG
Anti-HER2 scFv (high affinity) amino acid sequence
(SEQ ID NO: 16)
MDFQVQIFSFLLISASVIMSRGDIQMTQSPSSLSASVGDRVTITCRASQDVNTAVAWYQQKP
GKAPKLLIYSASFLYSGVPSRESGSRSGTDFTLTISSLQPEDFATYYCQQHYTTPPTFGQGT
KVEIKRTGSTSGSGKPGSGEGSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYYCSRWGGD
GFYAMDVWGQGTLVTVSS
Anti-HER2 scFv (low affinity) nucleotide sequence
(SEQ ID NO: 17)
ATGGATTTTCAGGTGCAGATTTTCAGCTTCCTGCTAATCAGTGCCTCAGTCATAATGTCCAG
AGGAGATATCCAGATGACCCAGTCCCCGAGCTCCCTGTCCGCCTCTGTGGGCGATAGGGTCA
CCATCACCTGCCGTGCCAGTCAGGATGTGAATACTGCTGTAGCCTGGTATCAACAGAAACCA
GGAAAAGCTCCGAAACTACTGATTTACTCGGCATCCTTCCTTGAGTCTGGAGTCCCTTCTCG
CTTCTCTGGATCTAGATCTGGGACGGATTTCACTCTGACCATCAGCAGTCTGCAGCCGGAAG
ACTTCGCAACTTATTACTGTCAGCAACATTATACTACTCCTCCCACGTTCGGACAGGGTACC
AAGGTGGAGATCAAACGCACTGGGTCTACATCTGGATCTGGGAAGCCGGGTTCTGGTGAGGG
TTCTGAGGTTCAGCTGGTGGAGTCTGGCGGTGGCCTGGTGCAGCCAGGGGGCTCACTCCGTT
TGTCCTGTGCAGCTTCTGGCTTCAACATTAAAGACACCTATATACACTGGGTGCGTCAGGCC
CCGGGTAAGGGCCTGGAATGGGTTGCAAGGATTTATCCTACGAATGGTTATACTAGATATGC
CGATAGCGTCAAGGGCCGTTTCACTATAAGCGCAGACACATCCAAAAACACAGCCTACCTGC
AGATGAACAGCCTGCGTGCTGAGGACACTGCCGTCTATTATTGTTCTAGATGGGGAGGGGAC
GGCTTCGTTGCTATGGACGTGTGGGGTCAAGGAACCCTGGTCACCGTCTCCTCG
Anti-HER2 scFv (low affinity) amino acid sequence
(SEQ ID NO: 18)
MDFQVQIFSFLLISASVIMSRGDIQMTQSPSSLSASVGDRVTITCRASQDVNTAVAWYQQKP
GKAPKLLIYSASFLESGVPSRFSGSRSGTDFTLTISSLQPEDFATYYCQQHYTTPPTFGQGT
KVEIKRTGSTSGSGKPGSGEGSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYYCSRWGGD
GFVAMDVWGQGTLVTVSS
Anti-TnMuc1 scFv nucleotide sequence
(SEQ ID NO: 19)
CAGGTGCAGCTGCAGCAGTCTGATGCCGAGCTCGTGAAGCCTGGCAGCAGCGTGAAGATCAG
CTGCAAGGCCAGCGGCTACACCTTCACCGACCACGCCATCCACTGGGTCAAGCAGAAGCCTG
AGCAGGGCCTGGAGTGGATCGGCCACTTCAGCCCCGGCAACACCGACATCAAGTACAACGAC
AAGTTCAAGGGCAAGGCCACCCTGACCGTGGACAGAAGCAGCAGCACCGCCTACATGCAGCT
GAACAGCCTGACCAGCGAGGACAGCGCCGTGTACTTCTGCAAGACCAGCACCTTCTTTTTCG
ACTACTGGGGCCAGGGCACAACCCTGACAGTGTCTAGCGGAGGCGGAGGATCTGGCGGCGGA
GGAAGTGGCGGAGGGGGATCTGAACTCGTGATGACCCAGAGCCCCAGCTCTCTGACAGTGAC
AGCCGGCGAGAAAGTGACCATGATCTGCAAGTCCTCCCAGAGCCTGCTGAACTCCGGCGACC
AGAAGAACTACCTGACCTGGTATCAGCAGAAACCCGGCCAGCCCCCCAAGCTGCTGATCTTT
TGGGCCAGCACCCGGGAAAGCGGCGTGCCCGATAGATTCACAGGCAGCGGCTCCGGCACCGA
CTTTACCCTGACCATCAGCTCCGTGCAGGCCGAGGACCTGGCCGTGTATTACTGCCAGAACG
ACTACAGCTACCCCCTGACCTTCGGAGCCGGCACCAAGCTGGAACTGAAG
Anti-TnMuc1 scFv amino acid sequence
(SEQ ID NO: 20)
QVQLQQSDAELVKPGSSVKISCKASGYTFTDHAIHWVKQKPEQGLEWIGHFSPGNTDIKYND
KFKGKATLTVDRSSSTAYMQLNSLTSEDSAVYFCKTSTFFFDYWGQGTTLTVSSGGGGSGGG
GSGGGGSELVMTQSPSSLTVTAGEKVTMICKSSQSLLNSGDQKNYLTWYQQKPGQPPKLLIF
WASTRESGVPDRFTGSGSGTDFTLTISSVQAEDLAVYYCQNDYSYPLTFGAGTKLELK
Anti-PSMA scFv nucleotide sequence
(SEQ ID NO: 21)
ATGGCCCTGCCTGTGACAGCCCTGCTGCTGCCTCTGGCTCTGCTGCTGCACGCCGCCAGACC
TGGATCTGACATTGTGATGACCCAGTCTCACAAATTCATGTCCACATCAGTAGGAGACAGGG
TCAGCATCATCTGTAAGGCCAGTCAAGATGTGGGTACTGCTGTAGACTGGTATCAACAGAAA
CCAGGACAATCTCCTAAACTACTGATTTATTGGGCATCCACTCGGCACACTGGAGTCCCTGA
TCGCTTCACAGGCAGTGGATCTGGGACAGACTTCACTCTCACCATTACTAACGTTCAGTCTG
AAGACTTGGCAGATTATTTCTGTCAGCAATATAACAGCTATCCTCTCACGTTCGGTGCTGGG
ACCATGCTGGACCTGAAAGGAGGCGGAGGATCTGGCGGCGGAGGAAGTTCTGGCGGAGGCAG
CGAGGTGCAGCTGCAGCAGAGCGGACCCGAGCTCGTGAAGCCTGGAACAAGCGTGCGGATCA
GCTGCAAGACCAGCGGCTACACCTTCACCGAGTACACCATCCACTGGGTCAAGCAGTCCCAC
GGCAAGAGCCTGGAGTGGATCGGCAATATCAACCCCAACAACGGCGGCACCACCTACAACCA
GAAGTTCGAGGACAAGGCCACCCTGACCGTGGACAAGAGCAGCAGCACCGCCTACATGGAAC
TGCGGAGCCTGACCAGCGAGGACAGCGCCGTGTACTATTGTGCCGCCGGTTGGAACTTCGAC
TACTGGGGCCAGGGCACAACCCTGACAGTGTCTAGCGCTAGCTCCGGA
Anti-PSMA scFv amino acid sequence
(SEQ ID NO: 22)
MALPVTALLLPLALLLHAARPGSDIVMTQSHKFMSTSVGDRVSIICKASQDVGTAVDWYQQK
PGQSPKLLIYWASTRHTGVPDRFTGSGSGTDFTLTITNVQSEDLADYFCQQYNSYPLTFGAG
TMLDLKGGGGSGGGGSSGGGSEVQLQQSGPELVKPGTSVRISCKTSGYTFTEYTIHWVKQSH
GKSLEWIGNINPNNGGTTYNQKFEDKATLTVDKSSSTAYMELRSLTSEDSAVYYCAAGWNED
YWGQGTTLTVSSASSG
Anti-EGFRvIII scFv nucleotide sequence
(SEQ ID NO: 23)
ATGGCCCTCCCTGTCACCGCCCTGCTGCTTCCGCTGGCTCTTCTGCTCCACGCCGCTCGGCC
CGAGATTCAGCTCGTGCAATCGGGAGCGGAAGTCAAGAAGCCAGGAGAGTCCTTGCGGATCT
CATGCAAGGGTAGCGGCTTTAACATCGAGGATTACTACATCCACTGGGTGAGGCAGATGCCG
GGGAAGGGACTCGAATGGATGGGACGGATCGACCCAGAAAACGACGAAACTAAGTACGGTCC
GATCTTCCAAGGCCATGTGACTATTAGCGCCGATACTTCAATCAATACCGTGTATCTGCAAT
GGTCCTCATTGAAAGCCTCAGATACCGCGATGTACTACTGTGCTTTCAGAGGAGGGGTCTAC
TGGGGACAGGGAACTACCGTGACTGTCTCGTCCGGCGGAGGCGGGTCAGGAGGTGGCGGCAG
CGGAGGAGGAGGGTCCGGCGGAGGTGGGTCCGACGTCGTGATGACCCAGAGCCCTGACAGCC
TGGCAGTGAGCCTGGGCGAAAGAGCTACCATTAACTGCAAATCGTCGCAGAGCCTGCTGGAC
TCGGACGGAAAAACGTACCTCAATTGGCTGCAGCAAAAGCCTGGCCAGCCACCGAAGCGCCT
TATCTCACTGGTGTCGAAGCTGGATTCGGGAGTGCCCGATCGCTTCTCCGGCTCGGGATCGG
GTACTGACTTCACCCTCACTATCTCCTCGCTTCAAGCAGAGGACGTGGCCGTCTACTACTGC
TGGCAGGGAACCCACTTTCCGGGAACCTTCGGCGGAGGGACGAAAGTGGAGATCAAG
Anti-EGFRvIII scFv amino acid sequence
(SEQ ID NO: 24)
MALPVTALLLPLALLLHAARPEIQLVQSGAEVKKPGESLRISCKGSGFNIEDYYIHWVRQMP
GKGLEWMGRIDPENDETKYGPIFQGHVTISADTSINTVYLQWSSLKASDTAMYYCAFRGGVY
WGQGTTVTVSSGGGGSGGGGSGGGGSGGGGSDVVMTQSPDSLAVSLGERATINCKSSQSLLD
SDGKTYLNWLQQKPGQPPKRLISLVSKLDSGVPDRFSGSGSGTDFTLTISSLQAEDVAVYYC
WQGTHFPGTFGGGTKVEIK
Anti-FAP scFv nucleotide sequence
(SEQ ID NO: 25)
CAAATTGTTCTCACCCAGTCTCCAGCGCTCATGTCTGCTTCTCCAGGGGAGAAGG+0TCACC
ATGACCTGCACTGCCAGCTCAAGTGTTAGTTACATGTACTGGTACCAGCA+0GAAGCCACGA
TCCTCCCCCAAACCCTGGATTTTTCTCACCTCCAACCTGGCTTCT+0GGAGTCCCTGCTCGC
TTCAGTGGCCGTGGGTCTGGGACCTCTTTCTCTCTCACAAT+0CAGCAGCATGGAGGCTGAA
GATGCTGCCACTTATTACTGCCAGCAGTGGAGTGG+0TTACCCACCCATCACATTCGGCTCG
GGGACAAAGTTGGAAATAAAAGGTGGAGG+0TGGCAGCGGAGGAGGTGGGTCCGGCGGTGGA
GGAAGCCAGGTCCAACTGCAGC+0AGCCTGGGGCTGAACTGGTAAAGCCTGGGGCTTCAGTG
AAGTTGTCCTGCAAGG+0CGTCTGGCTACACCATCACCAGCTACTCTCTGCACTGGGTGAAG
CAGAGGCCTGG+0ACAAGGCCTTGAGTGGATTGGAGAGATTAATCCTGCCAATGGTGATCAT
AACTTC+0AGTGAGAAGTTCGAGATCAAGGCCACACTGACTGTAGACAGCTCCTCCAACACA
+0GCATTCATGCAACTCAGCAGGCTGACATCTGAGGACTCTGCGGTCTATTACTGTA+0CAA
GATTGGACGATAGTAGGTTCCACTGGTACTTCGATGTCTGGGGCGCAGGGA+0CCACGGTCA
CCGTCTCCTCA
Anti-FAP scFv amino acid sequence
(SEQ ID NO: 26)
QIVLTQSPALMSASPGEKVTMTCTASSSVSYMYWYQQKPRSSPKPWIFLTSNLASGV+0PAR
FSGRGSGTSFSLTISSMEAEDAATYYCQQWSGYPPITFGSGTKLEIKGGGGSGG+0GGSGGG
GSQVQLQQPGAELVKPGASVKLSCKASGYTITSYSLHWVKQRPGQGLEWIG+0EINPANGDH
NFSEKFEIKATLTVDSSSNTAFMQLSRLTSEDSAVYYCTRLDDSRFHW+0YFDVWGAGTTVT
VSS
Additional CAR domain sequences include:
Mouse CD8 Leader
(SEQ ID NO: 27)
MASPLTRFLSLNLLLLGESIILGSGEA
Mouse CD8 Leader
(SEQ ID NO: 28)
ATGGCCTCACCCCTGACCAGATTCCTGTCACTGAACCTGCTGCTGCTGGGCGAGTCAATCAT
CCTGGGCTCAGGCGAGGCC
Mouse anti-mCD19 scFv
(SEQ ID NO: 29)
DIQMTQSPASLSTSLGETVTIQCQASEDIYSGLAWYQQKPGKSPQLLIYGASDLQDGVPSRF
SGSGSGTQYSLKITSMQTEDEGVYFCQQGLTYPRTFGGGTKLELKGGGGSGGGGSGGGGSEV
QLQQSGAELVRPGTSVKLSCKVSGDTITFYYMHFVKQRPGQGLEWIGRIDPEDESTKYSEKF
KNKATLTADTSSNTAYLKLSSLTSEDTATYFCIYGGYYFDYWGQGVMVTVSS
Mouse anti-CD19 scFv
(SEQ ID NO: 30)
GACATCCAGATGACCCAGAGCCCTGCCAGCCTGTCTACCAGCCTGGGCGAGACAGTGACCAT
CCAGTGTCAGGCCAGCGAGGACATCTACTCTGGCCTGGCTTGGTATCAGCAGAAGCCCGGCA
AGAGCCCTCAGCTGCTGATCTACGGCGCCAGCGACCTGCAGGACGGCGTGCCTAGCAGATTC
AGCGGCAGCGGCTCCGGAACCCAGTACAGCCTGAAGATCACCAGCATGCAGACCGAGGACGA
GGGCGTGTACTTCTGCCAGCAAGGCCTGACCTACCCTAGAACCTTCGGAGGAGGCACCAAGC
TGGAACTGAAGGGCGGAGGCGGAAGTGGAGGCGGAGGATCTGGCGGCGGAGGCTCTGAAGTG
CAGCTGCAGCAGTCTGGCGCTGAACTGGTCCGGCCTGGCACTAGCGTGAAGCTGTCCTGCAA
GGTGTCCGGCGACACCATCACCTTCTACTACATGCACTTCGTGAAGCAGAGGCCAGGACAGG
GCCTGGAATGGATCGGCAGAATCGACCCTGAGGACGAGAGCACCAAGTACAGCGAGAAGTTC
AAGAACAAGGCCACCCTGACCGCCGACACCAGCAGCAACACCGCCTACCTGAAGCTGTCTAG
CCTGACCTCCGAGGACACCGCCACCTACTTTTGCATCTACGGCGGCTACTACTTCGACTACT
GGGGCCAGGGCGTGATGGTCACCGTGTCCAGC
Mouse CD8 Hinge
(SEQ ID NO: 31)
LQKVNSTTTKPVLRTPSPVHPTGTSQPQRPEDCRPRGSVKGTGLDFACDIY
Mouse CD8 Hinge
(SEQ ID NO: 32)
CTGCAGAAGGTGAACTCAACCACCACCAAGCCCGTGCTGAGAACCCCCTCACCCGTGCACCC
CACCGGCACCTCACAGCCCCAGAGACCCGAGGACTGCAGACCCAGAGGCTCAGTGAAGGGCA
CCGGCCTGGACTTCGCCTGCGACATCTAC
Mouse CD28 TM
(SEQ ID NO: 33)
FWALVVVAGVLFCYGLLVTVALCVIWT
Mouse CD28 TM
(SEQ ID NO: 34)
TTTTGGGCACTGGTCGTGGTTGCTGGAGTCCTGTTTTGTTATGGCTTGCTAGTGACAGTGGC
TCTTTGTGTTATCTGGACA
Mouse CD28 ICD
(SEQ ID NO: 35)
NSRRNRLLQSDYMNMTPRRPGLTRKPYQPYAPARDFAAYRP
Mouse CD28 ICD
(SEQ ID NO: 36)
AATAGTAGAAGGAACAGACTCCTTCAAAGTGACTACATGAACATGACTCCCCGGAGGCCTGG
GCTCACTCGAAAGCCTTACCAGCCCTACGCCCCTGCCAGAGACTTTGCAGCGTACCGCCCC
Mouse CD3z
(SEQ ID NO: 37)
KFSRSAETAANLQDPNQLYNELNLGRREEYDVLEKKRARDPEMGGKQQRRRNPQEGVYNALQ
KDKMAEAYSEIGTKGERRRGKGHDGLYQGLSTATKDTYDALHMQTLAPR
Mouse CD3z
(SEQ ID NO: 38)
AAGTTCTCAAGATCAGCCGAGACCGCCGCCAACCTGCAGGACCCCAACCAGCTGTACAACGA
GCTGAACCTGGGCAGAAGAGAGGAGTACGACGTGCTGGAGAAGAAGAGAGCCAGAGACCCCG
AGATGGGCGGCAAGCAGCAGAGAAGAAGAAACCCCCAGGAGGGCGTGTACAACGCCCTGCAG
AAGGACAAGATGGCCGAGGCCTACTCAGAGATCGGCACCAAGGGCGAGAGAAGAAGAGGCAA
GGGCCACGACGGCCTGTACCAGGGCCTGTCAACCGCCACCAAGGACACCTACGACGCCCTGC
ACATGCAGACCCTGGCCCCCAGA
Human CD8 leader
(SEQ ID NO: 39)
MALPVTALLLPLALLLHAARP
Human CD8 leader
(SEQ ID NO: 40)
ATGGCCTTACCAGTGACCGCCTTGCTCCTGCCGCTGGCCTTGCTGCTCCACGCCGCCAGGCC
G
Human anti-CD19 scFv
(SEQ ID NO: 41)
EIVMTQSPATLSLSPGERATLSCRASQDISKYLNWYQQKPGQAPRLLIYHTSRLHSGIPARF
SGSGSGTDYTLTISSLQPEDFAVYFCQQGNTLPYTFGQGTKLEIKGGGGSGGGGSGGGGSQV
QLQESGPGLVKPSETLSLTCTVSGVSLPDYGVSWIRQPPGKGLEWIGVIWGSETTYYQSSLK
SRVTISKDNSKNQVSLKLSSVTAADTAVYYCAKHYYYGGSYAMDYWGQGTLVTVSS
Human anti-CD19 scFv
(SEQ ID NO: 42)
GAAATTGTGATGACCCAGTCACCCGCCACTCTTAGCCTTTCACCCGGTGAGCGCGCAACCCT
GTCTTGCAGAGCCTCCCAAGACATCTCAAAATACCTTAATTGGTATCAACAGAAGCCCGGAC
AGGCTCCTCGCCTTCTGATCTACCACACCAGCCGGCTCCATTCTGGAATCCCTGCCAGGTTC
AGCGGTAGCGGATCTGGGACCGACTACACCCTCACTATCAGCTCACTGCAGCCAGAGGACTT
CGCTGTCTATTTCTGTCAGCAAGGGAACACCCTGCCCTACACCTTTGGACAGGGCACCAAGC
TCGAGATTAAAGGTGGAGGTGGCAGCGGAGGAGGTGGGTCCGGCGGTGGAGGAAGCCAGGTC
CAACTCCAAGAAAGCGGACCGGGTCTTGTGAAGCCATCAGAAACTCTTTCACTGACTTGTAC
TGTGAGCGGAGTGTCTCTCCCCGATTACGGGGTGTCTTGGATCAGACAGCCACCGGGGAAGG
GTCTGGAATGGATTGGAGTGATTTGGGGCTCTGAGACTACTTACTACCAATCATCCCTCAAG
TCACGCGTCACCATCTCAAAGGACAACTCTAAGAATCAGGTGTCACTGAAACTGTCATCTGT
GACCGCAGCCGACACCGCCGTGTACTATTGCGCTAAGCATTACTATTATGGCGGGAGCTACG
CAATGGATTACTGGGGACAGGGTACTCTGGTCACCGTGTCCAGC
Human CD8 Hinge
(SEQ ID NO: 43)
TTTPAPRPPTPAPTIASQPLSLRPEACRPAAGGAVHTRGLDFACD
Human CD8 Hinge
(SEQ ID NO: 44)
ACCACGACGCCAGCGCCGCGACCACCAACACCGGCGCCCACCATCGCGTCGCAGCCCCTGTC
CCTGCGCCCAGAGGCGTGCCGGCCAGCGGCGGGGGGCGCAGTGCACACGAGGGGGCTGGACT
TCGCCTGTGAT
Human CD28 TM
(SEQ ID NO: 45)
FWVLVVVGGVLACYSLLVTVAFIIFWV
Human CD28 TM
(SEQ ID NO: 46)
TTTTGGGTGCTGGTGGTGGTTGGTGGAGTCCTGGCTTGCTATAGCTTGCTAGTAACAGTGGC
CTTTATTATTTTCTGGGTG
Human CD28 ICD
(SEQ ID NO: 47)
RSKRSRLLHSDYMNMTPRRPGPTRKHYQPY APPRDFAAYRS
Human CD28 ICD
(SEQ ID NO: 48)
AGGAGTAAGAGGAGCAGGCTCCTGCACAGTGACTACATGAACATGACTCCCCGCCGCCCCGG
GCCCACCCGCAAGCATTACCAGCCCTATGCCCCACCACGCGACTTCGCAGCCTATCGCTCC
Human CD3z ICD
(SEQ ID NO: 49)
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Human CD3z ICD
(SEQ ID NO: 50)
AGAGTGAAGTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACGTGGCCGGG
ACCCTGAGATGGGGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCC
TTCACATGCAGGCCCTGCCCCCTCGC
Mouse CD8 Leader
(SEQ ID NO: 51)
MASPLTRFLSLNLLLLGESIILGSGEA
Mouse CD8 Leader
(SEQ ID NO: 52)
ATGGCCAGCCCCCTGACCAGATTCCTGAGCCTGAACCTGCTGCTGCTGGGCGAGAGCATCAT
CCTGGGCAGCGGCGAGGCC
A03 mouse anti-Meso scFv
(SEQ ID NO: 53)
AQVQLQESGPGLVKPSQTLSLTCTVSGGSISSGGYYWSWIRQHPGKGLEWIGYIYYSGSTYY
NPSLKSRVTISVDTSKNQFSLKLSSVTAADTAVYYCARFDYGDFYDAFDIWGQGTMVTVSSG
GGGSGGGGSGGGGSEIVLTQSPSSLSASVGDRVTITCRASQGISSALAWYQQKPGKAPKLLI
YDASSLESGVPSRFSGSGSGTDFTLTISSLQPEDFATYYCQQFNSYPITFGQGTRLEIKR
A03 mouse anti-Meso scFv
(SEQ ID NO: 54)
GCCCAGGTGCAGCTGCAGGAGTCGGGCCCAGGACTGGTGAAGCCTTCACAGACCCTGTCCCT
CACCTGCACTGTCTCTGGTGGCTCCATCAGCAGTGGTGGTTACTACTGGAGCTGGATCCGCC
AGCACCCAGGGAAGGGCCTGGAGTGGATTGGGTACATCTATTACAGTGGGAGCACCTACTAC
AACCCGTCCCTCAAGAGTCGAGTTACCATATCAGTAGACACGTCCAAGAACCAGTTCTCCCT
GAAGCTGAGCTCTGTGACCGCTGCGGACACGGCCGTGTATTACTGTGCGAGATTTGACTACG
GTGACTTCTATGATGCTTTTGATATCTGGGGCCAAGGGACAATGGTCACCGTCTCTTCAGGT
GGTGGTGGTAGCGGCGGCGGCGGCTCTGGTGGTGGTGGATCCGAAATTGTGTTGACGCAGTC
TCCATCCTCCCTGTCTGCATCTGTAGGAGACAGAGTCACCATCACTTGCCGGGCAAGTCAGG
GCATTAGCAGTGCTTTAGCCTGGTATCAGCAGAAACCAGGGAAAGCTCCTAAGCTCCTGATC
TATGATGCCTCCAGTTTGGAAAGTGGGGTCCCATCAAGGTTCAGCGGCAGTGGATCTGGGAC
AGATTTCACTCTCACCATCAGCAGCCTGCAGCCTGAAGATTTTGCAACTTATTACTGTCAAC
AGTTTAATAGTTACCCGATCACCTTCGGCCAAGGGACACGACTGGAGATTAAACGT
Mouse CD8 Hinge
(SEQ ID NO: 55)
LQKVNSTTTKPVLRTPSPVHPTGTSQPQRPEDCRPRGSVKGTGLDFACDIY
Mouse CD8 Hinge
(SEQ ID NO: 56)
CTGCAGAAGGTGAACAGCACCACCACCAAGCCCGTGCTGAGAACCCCCAGCCCCGTGCACCC
CACCGGCACCAGCCAGCCCCAGAGACCCGAGGACTGCAGACCCAGAGGCAGCGTGAAGGGCA
CCGGCCTGGACTTCGCCTGCGACATCTAC
Mouse CD8 TM
(SEQ ID NO: 57)
IWAPLAGICVALLLSLIITLI
Mouse CD8 TM
(SEQ ID NO: 58)
ATCTGGGCCCCCCTGGCCGGCATCTGCGTGGCCCTGCTGCTGAGCCTGATCATCACCCTGAT
C
Mouse 4-1BB ICD
(SEQ ID NO: 59)
KWIRKKFPHIFKQPFKKTTGAAQEEDACSCRCPQEEEGGGGGYEL
Mouse 4-1BB ICD
(SEQ ID NO: 60)
AAGTGGATCAGAAAGAAGTTCCCCCACATCTTCAAGCAGCCCTTCAAGAAGACCACCGGCGC
CGCCCAGGAGGAGGACGCCTGCAGCTGCAGATGCCCCCAGGAGGAGGAGGGCGGCGGCGGCG
GCTACGAGCTG
Mouse CD3z ICD
(SEQ ID NO: 61)
KFSRSAETAANLQDPNQLYNELNLGRREEYDVLEKKRARDPEMGGKQQRRRNPQEGVYNALQ
KDKMAEAYSEIGTKGERRRGKGHDGLYQGLSTATKDTYDALHMQTLAPR
Mouse CD3z ICD
(SEQ ID NO: 62)
AAGTTCAGCAGAAGCGCCGAGACCGCCGCCAACCTGCAGGACCCCAACCAGCTGTACAACGA
GCTGAACCTGGGCAGAAGAGAGGAGTACGACGTGCTGGAGAAGAAGAGAGCCAGAGACCCCG
AGATGGGCGGCAAGCAGCAGAGAAGAAGAAACCCCCAGGAGGGCGTGTACAACGCCCTGCAG
AAGGACAAGATGGCCGAGGCCTACAGCGAGATCGGCACCAAGGGCGAGAGAAGAAGAGGCAA
GGGCCACGACGGCCTGTACCAGGGCCTGAGCACCGCCACCAAGGACACCTACGACGCCCTGC
ACATGCAGACCCTGGCCCCCAGA
Human CD8 Leader
(SEQ ID NO: 63)
MALPVTALLLPLALLLHAARP
Human CD8 Leader
(SEQ ID NO: 64)
ATGGCCCTCCCTGTCACCGCCCTGCTGCTTCCGCTGGCTCTTCTGCTCCACGCCGC
TCGGCCC
Human M5 scFv
(SEQ ID NO: 65)
QVQLVQSGAEVEKPGASVKVSCKASGYTFTDYYMHWVRQAPGQGLEWMGWINPNSGGTNYAQ
KFQGRVTMTRDTSISTAYMELSRLRSDDTAVYYCASGWDFDYWGQGTLVTVSSGGGGSGGGG
SGGGGSGGGGSDIVMTQSPSSLSASVGDRVTITCRASQSIRYYLSWYQQKPGKAPKLLIYTA
SILQNGVPSRFSGSGSGTDFTLTISSLQPEDFATYYCLQTYTTPDFGPGTKVEIK
Human M5 scFv
(SEQ ID NO: 66)
CAAGTCCAACTCGTTCAATCAGGCGCAGAAGTCGAAAAGCCCGGAGCATCAGTCAAAGTCTC
TTGCAAGGCTTCCGGCTACACCTTCACGGACTACTACATGCACTGGGTGCGCCAGGCTCCAG
GCCAGGGACTGGAGTGGATGGGATGGATCAACCCGAATTCCGGGGGAACTAACTACGCCCAG
AAGTTTCAGGGCCGGGTGACTATGACTCGCGATACCTCGATCTCGACTGCGTACATGGAGCT
CAGCCGCCTCCGGTCGGACGATACCGCCGTGTACTATTGTGCGTCGGGATGGGACTTCGACT
ACTGGGGGCAGGGCACTCTGGTCACTGTGTCAAGCGGAGGAGGTGGATCAGGTGGAGGTGGA
AGCGGGGGAGGAGGTTCCGGCGGCGGAGGATCAGATATCGTGATGACGCAATCGCCTTCCTC
GTTGTCCGCATCCGTGGGAGACAGGGTGACCATTACTTGCAGAGCGTCCCAGTCCATTCGGT
ACTACCTGTCGTGGTACCAGCAGAAGCCGGGGAAAGCCCCAAAACTGCTTATCTATACTGCC
TCGATCCTCCAAAACGGCGTGCCATCAAGATTCAGCGGTTCGGGCAGCGGGACCGACTTTAC
CCTGACTATCAGCAGCCTGCAGCCGGAAGATTTCGCCACGTACTACTGCCTGCAAACCTACA
CCACCCCGGACTTCGGACCTGGAACCAAGGTGGAGATCAAG
Human CD8 Hinge
(SEQ ID NO: 67)
TTTPAPRPPTPAPTIASQPLSLRPEACRPAAGGAVHTRGLDFACD
Human CD8 Hinge
(SEQ ID NO: 68)
ACCACTACCCCAGCACCGAGGCCACCCACCCCGGCTCCTACCATCGCCTCCCAGCCTCTGTC
CCTGCGTCCGGAGGCATGTAGACCCGCAGCTGGTGGGGCCGTGCATACCCGGGGTCTTGACT
TCGCCTGCGAT
Human CD8 TM
(SEQ ID NO: 69)
IYIWAPLAGTCGVLLLSLVITLYC
Human CD8 TM
(SEQ ID NO: 70)
ATCTACATTTGGGCCCCTCTGGCTGGTACTTGCGGGGTCCTGCTGCTTTCACTCGTGATCAC
TCTTTACTGT
Human 4-1BB ICD
(SEQ ID NO: 71)
KRGRKKLLYIFKQPFMRPVQTTQEEDGCSCRFPEEEEGGCEL
Human 4-1BB ICD
(SEQ ID NO: 72)
AAGCGCGGTCGGAAGAAGCTGCTGTACATCTTTAAGCAACCCTTCATGAGGCCTGTGCAGAC
TACTCAAGAGGAGGACGGCTGTTCATGCCGGTTCCCAGAGGAGGAGGAAGGCGGCTGCGAAC
TG
Human CD3z ICD
(SEQ ID NO: 73)
RVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Human CD3z ICD
(SEQ ID NO: 74)
CGCGTGAAATTCAGCCGCAGCGCAGATGCTCCAGCCTACAAGCAGGGGCAGAACCAGCTCTA
CAACGAACTCAATCTTGGTCGGAGAGAGGAGTACGACGTGCTGGACAAGCGGAGAGGACGGG
ACCCAGAAATGGGCGGGAAGCCGCGCAGAAAGAATCCCCAAGAGGGCCTGTACAACGAGCTC
CAAAAGGATAAGATGGCAGAAGCCTATAGCGAGATTGGTATGAAAGGGGAACGCAGAAGAGG
CAAAGGCCACGACGGACTGTACCAGGGACTCAGCACCGCCACCAAGGACACCTATGACGCTC
TTCACATGCAGGCCCTGCCGCCTCGG

### C. Nucleic Acids and Expression Vectors

The present disclosure provides an isolated nucleic acid comprising a first nucleotide sequence encoding the first CAR and a second nucleotide sequence encoding the second CAR as described herein. That is, the invention provides an isolated nucleic acid comprising: (a) a first nucleotide sequence encoding a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second nucleotide sequence encoding a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In one embodiment, the isolated nucleic acid comprises: (a) a first nucleotide sequence encoding a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second nucleotide sequence encoding a second CAR comprising: (i) a second extracellular antigen binding domain having affinity for a tumor antigen, (ii) a second transmembrane domain, and (iii) a second intracellular domain comprising at least one costimulatory domain and a signaling domain; wherein the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family α-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

In certain embodiments, the first nucleotide sequence is situated 5′ to the second nucleotide sequence. In certain embodiments, the first nucleotide sequence is situated 3′ to the second nucleotide sequence. In certain embodiments, the isolated nucleic acid further comprises a linker nucleotide sequence situated between the first nucleotide sequence and the second nucleotide sequence. A linker for use in the present disclosure allows for multiple proteins to be encoded by the same nucleic acid (e.g., a multicistronic or bicistronic sequence), which are translated as a polyprotein that is dissociated into separate protein components. In certain embodiments, the nucleic acid comprises from 5′ to 3′ the first nucleotide sequence, the linker, and the second nucleotide sequence. In certain embodiments, the nucleic acid comprises from 5′ to 3′ the second nucleotide sequence, the linker, and the first nucleotide sequence.

In some embodiments, the linker comprises a nucleic acid sequence that encodes an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a ribosome slip sequence which are also known in the art as a sequence encoding a self-cleaving peptide. As used herein, a “self-cleaving peptide” or “2A peptide” refers to an sequence that allow multiple proteins to be encoded as polyproteins, which dissociate into component proteins upon translation. Use of the term “self-cleaving” is not intended to imply a proteolytic cleavage reaction. Various ribosome slip sequences, i.e., self-cleaving or 2A peptides, are known to those of skill in the art, including, without limitation, those found in members of the Picomaviridae virus family, e.g., foot-and-mouth disease virus (FMDV), equine rhinitis A virus (ERAV0, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate ribosome slip sequence for use in the present invention.

In some embodiments, the construct includes a linker that optionally, further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding a Furin cleavage site and F2A, a linker comprising a nucleic acid sequence encoding a Furin cleavage site and E2A, a linker comprising a nucleic acid sequence encoding a Furin cleavage site and P2A, a linker comprising a nucleic acid sequence encoding a Furin cleavage site and T2A. Those of skill in the art would be able to select the appropriate combination for use in the present invention. In such embodiments, the linker may further comprise a spacer sequence between the Furin cleavage site and the 2A peptide. In some embodiments, the linker comprises a Furin cleavage site 5′ to a 2A peptide. In some embodiments, the linker comprises a 2A peptide 5′ to a Furin cleavage site. Various spacer sequences are known in the art, including, without limitation, glycine serine (GS) spacers (also known as GS linkers). Those of skill in the art would be able to select the appropriate spacer sequence for use in the present invention.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid encoding an exogenous CAR is operably linked to a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lac repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha (aka EF-1α or EF1α) promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is used for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding for a CAR. In some embodiments, the expression vector (e.g., lentiviral vector) will comprise additional elements that will aid in the functional expression of the CAR encoded therein. In some embodiments, an expression vector comprising a nucleic acid encoding for a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15:9053. Introducing RNA comprising a nucleotide sequence encoding a CAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of a CAR as described herein, e.g., in a mammalian cell. In some embodiments, a nucleic acid of the present disclosure provides for amplification of the CAR-encoding nucleic acid and/or vector. In some embodiments, a cell comprising the isolated nucleic acid and/or vector is provided, wherein the cell is selected from a bacterial cell, a fungal cell, a yeast cell, an insect cell, an animal cell, a mammalian cell, and a human cell. In some embodiments, the cell is a mammalian or human cell and is an immune cell or precursor cell thereof, such as a T cell.

### D. Modified Immune Cells

The present invention further provides a modified immune cell or precursor cell thereof engineered to express the first CAR and the second CAR of the invention as described herein. That is, the modified cell is an immune cell or precursor cell thereof engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In one aspect, the modified cell is an immune cell or precursor cell thereof engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In certain embodiments, the modified immune cell or precursor cell thereof selected from a T cell (including, but not limited to, e.g., a natural killer T (NKT) cell and a gamma-delta T cell), a natural killer (NK) cell, and a macrophage). In certain embodiments, the modified cell is an autologous cell. In certain embodiments, the modified cell is an autologous cell obtained from a human subject. In certain embodiments, the modified cell is a T cell.

In certain embodiments, the modified cell of the invention is further engineered to express one or more additional receptors. In some embodiments, the modified cell is further engineered to express a switch receptor. In certain embodiments, the modified cell is further engineered to express a dominant negative receptor. In some embodiments, the modified cell is further engineered to express a switch receptor and a dominant negative receptor. As used herein, a “switch receptor” is a chimeric molecule that is able to switch a negative signal to a positive signal for enhancement of an immune response. See, e.g., WO2013019615A2 and WO2016122738A1. A switch receptor is a chimeric protein comprising an extracellular domain of a first protein or fragment thereof associated with a negative signal, and an intracellular domain of a second protein or fragment thereof associated with a positive signal. When expressed in a cell, a switch receptor converts a negative signal generated by the binding of an immunosuppressive ligand into a positive signal generated by the intracellular signaling domain of a costimulatory molecule. An example of a protein associated with a negative signal includes but is not limited to CTLA-4, TGFβRII, PD-1, VSIG8, VSIG3, BTLA, and TIM-3. An example of a protein associated with a positive signal includes but is not limited to CD28, 4-1BB, IL12β1, IL12Rβ2, IL2, IL19, CD2, ICOS, CD27 and the like.

Switch receptors comprise an extracellular domain of a first receptor and an intracellular domain of a second receptor, linked by a transmembrane domain. The transmembrane domain of a switch receptor is typically derived from the first receptor or from the second receptor, but may be any suitable transmembrane domain. In some embodiments, the first receptor and the second receptor are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R. In some embodiments, the switch receptor is a TGFβR-IL12R receptor, a TGFβR-CD28 receptor, a TGFβR-OX40 receptor, a PD1-IL12R receptor, a PD1-CD28 receptor, a PD1-ICOS receptor, a PD1-CD27 receptor, a BTLA-CD28 receptor, a BTLA-ICOS receptor, a BTLA-CD27 receptor, a IFNγ-CD28 receptor, a IFNγ-OX40 receptor, or a IFNγ-IL12R receptor.

In some embodiments, the switch receptor is selected from the group consisting of TGFβR/IL12R, TGFβR/CD28, TGFβR/OX40, PD1/IL12R, PD1/CD28, PD1/ICOS, PD1/CD27, BTLA/CD28, BTLA/ICOS and BTLA/CD27, IFNγ/CD28, IFNγ/OX40, and IFNγ/IL12R. In some embodiments, the switch receptor is selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1^{A132L}-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, VISTA-IL-9R, TIGIT-IL-9R, TGFβRII-CD27, TGFβRII-CD28, TGFβRII-4-1BB, TGFβRII-ICOS, TGFβRII-IL12Rβ1, and TGFβRII-IL12Rβ2. In some embodiments, the switch receptor comprises an extracellular domain of a signaling protein associated with a negative signal, a transmembrane domain, and an intracellular domain of a signaling protein associated with a positive signal. In some embodiments, the transmembrane domain of the switch receptor is selected from the transmembrane domain of the protein associated with a negative signal or the transmembrane domain of the protein associated with the negative signal. In some embodiments, the transmembrane domain of the switch receptor is selected from a transmembrane domain of a protein selected from the group consisting of CTLA4, PD-1, TGFβRII, VSIG8, VSIG3, BTLA, VISTA, TIGIT, IL-9R, IL-2R, TIM-3, CD28, 4-1BB, IL12ROI, IL12Rβ2, CD2, ICOS, and CD27.

Dominant-negative (DN) mutants represent an important class of mutation in which a DN mutant receptor interferes with the function of the wild-type (WT) version of the receptor. As used herein dominant negative receptor is (a) a truncated variant of a wild-type receptor, (b) a variant of a wild-type protein comprising an extracellular domain, a transmembrane domain, and substantially lacking an intracellular signaling domain; or (c) an extracellular domain of a signaling protein associated with a negative signal, and a transmembrane domain. In some embodiments, the dominant negative receptor is a variant of a protein associated with a negative signal. In some embodiments, the dominant negative receptor is selected from a CTLA-4, a TGFβRII, a PD-1, a VSIG8, a VSIG3, a BTLA, and a TIM-3 dominant negative receptor. In some embodiments, the dominant negative receptor is a dominant negative TGF-β Receptor (dnTGFβR). See, e.g., WO2016122738A1.

In some embodiments, the modified cell comprises a vector encoding the first CAR and the second CAR. In some embodiments, the modified cell comprises a vector encoding the first CAR, the second CAR, a switch receptor, and a dominant negative receptor. In certain embodiments, the vector is a retroviral vector or a lentiviral vector.

The modified cell of the present invention may be administered to a subject as a population of modified cells. Upon administration to a subject, such as a mammal or a human, the modified cells exhibit expansion in peripheral blood of the subject. In certain embodiments, the expansion is at least 10-fold, at least 100-fold, or at least 1000-fold. In certain embodiments, the modified cells are detectable for at least 24 months after the cells are administered. In certain embodiments, the population of modified cells comprises T cells and at least 30% or at least 40% of the population of modified cells at day 7 post-administration or beyond are phenotypically central memory T cells.

### E. Methods of Treatment

The present invention comprises the use of CD19 antigen-driven expansion of CAR-expressing immune cells (e.g., CAR T cells) in the peripheral blood (PB) of a patient which safely increases the therapeutic index of adoptive cell therapy targeting solid tumors. This is achieved using a dual CAR approach, wherein a subject receives an immune cell (i.e., a plurality of immune cells, e.g. a plurality of T cells) engineered to express a first CAR targeting CD19 antigen and a second CAR targeting a tumor antigen other than CD19 (i.e., dual CAR T cells; dual CAR immune cells). The CD19 antigen in the subject drives dual CAR T cell expansion (or CAR immune cell expansion). CD19 antigen is present in the subject endogenously, e.g., on CD19-expressing B cells, and/or is provided exogenously, e.g., as CD19 antigen protein, as a cell expressing CD19 antigen, or as a nucleic acid comprising a nucleotide sequence encoding CD19 antigen. Compared to cells expressing a single CAR targeting a tumor antigen, the dual CAR T cell approach (dual CAR immune cell) of the present invention surprisingly enhances CAR T cell expansion in vivo in both peripheral blood and in organs of the subject, enhances and accelerates tumor cell killing in vitro and in vivo (e.g., solid tumor cell killing and tumor size reduction), increases cytotoxic cytokine expression, and lowers on-target off-tumor cytotoxicity. Importantly, boosting and anti-tumor efficacy does not require lymphodepletion. Additionally, the boosted dual CAR T cells of the present invention are predominately an effector memory or a central memory phenotype and express only one or two exhaustion markers. Importantly, these unexpected effects are not mere additive effects of combining two CARs and the present invention addresses a long unmet need for treating solid tumors with adoptive cell therapy.

The modified cell (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a population of modified cells of the present invention, wherein the cells are immune cells or precursor cells thereof (e.g., T cells). In one aspect, the invention provides a method of treating cancer in a subject in need thereof, comprising administering to the subject a population of modified cells, wherein the cells are immune cells or precursor cells thereof, and wherein the cells are engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, wherein the tumor antigen is not CD19, (ii) a second transmembrane domain, and (iii) a second intracellular domain comprising at least one costimulatory domain and a signaling domain.

In one embodiment, the invention provides a method of treating cancer in a subject in need thereof, comprising administering to the subject a population of modified cells, wherein the cells are immune cells or precursor cells thereof, and wherein the cells are engineered to express: (a) a first chimeric antigen receptor (CAR) comprising: (i) a first extracellular antigen binding domain having affinity for CD19, (ii) a first transmembrane domain, and (iii) a first intracellular domain comprising at least one costimulatory domain and a signaling domain; and (b) a second CAR comprising: (i) a second extracellular antigen binding domain having affinity for a tumor antigen, (ii) a second transmembrane domain, and (iii) a second intracellular domain comprising at least one costimulatory domain and a signaling domain; wherein the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family α-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

In some embodiments, the modified cells are further engineered to express a switch receptor described herein, a dominant negative receptor described herein, or both.

In some embodiments, the modified cells comprise a vector encoding the first CAR and the second CAR. In some embodiments, the modified cells comprise a vector encoding the first CAR, the second CAR, a switch receptor, and a dominant negative receptor. In certain embodiments, the vector is a retroviral vector or a lentiviral vector.

In some embodiments, the population of modified cells comprises about 1×10⁶ to about 1×10⁹ cells. Upon administration to a subject, such as a mammal or a human, the modified cells exhibit expansion in peripheral blood of the subject. In certain embodiments, the expansion is at least 10-fold, at least 100-fold, or at least 1000-fold. In certain embodiments, the modified cells are detectable for at least 24 months after the cells are administered. In certain embodiments, the population of modified cells comprises T cells and at least 30% or at least 40% of the population of modified cells at day 7 post-administration or beyond are phenotypically central memory T cells.

In certain embodiments, the method further comprises administering a CD19 antigen to the subject. In some embodiments, administering the CD19 antigen comprises administered a CD19 antigen protein, a cell expressing CD19 antigen, or a nucleic acid comprising a nucleotide sequence encoding CD19 antigen. In some embodiments, administering the CD19 antigen comprises administering a vector comprising a nucleotide sequence encoding the CD19 antigen or a cell engineered to express the CD19 antigen. The CD19 antigen comprises a CD19 extracellular domain or antigenic fragment thereof. The CD19 antigen is administered prior to, concurrently with, or after the administration of the population of modified cells. In some embodiments, the vector comprising a nucleotide sequence encoding the CD19 antigen is an adenoviral vector. Any CD19 antigen (i.e., full-length CD19 or extracellular antigenic fragment thereof) may be used. In some embodiments, the CD19 antigen may be encoded by the following nucleotide sequence and comprises the following amino acid sequence:

CD19 antigen nucleotide sequence
(SEQ ID NO: 75)
AGGCCCCAGAAGTCCTTACTGGTGGAGGTAGAAGAGGGAGGCAAT
GTTGTGCTGCCATGCCTCCCGGACTCCTCACCTGTCTCTTCTGAG
AAGCTGGCTTGGTATCGAGGTAACCAGTCAACACCCTTCCTGGAG
CTGAGCCCCGGGTCCCCTGGCCTGGGATTGCACGTGGGGTCCCTG
GGCATCTTGCTAGTGATTGTCAATGTCTCAGACCATATGGGGGGC
TTCTACCTGTGCCAGAAGAGGCCCCCTTTCAAGGACATCTGGCAG
CCTGCCTGGACAGTGAACGTGGAGGATAGTGGGGAGATGTTCCGG
TGGAATGCTTCAGACGTCAGGGACCTGGACTGTGACCTAAGGAAC
AGGTCCTCTGGGAGCCACAGGTCCACTTCTGGTTCCCAGCTGTAT
GTGTGGGCTAAAGACCATCCTAAGGTCTGGGGAACAAAGCCTGTA
TGTGCCCCTCGGGGGAGCAGTTTGAATCAGAGTCTAATCAACCAA
GATCTCACTGTGGCACCCGGCTCCACACTTTGGCTGTCCTGTGGG
GTACCCCCTGTCCCAGTGGCCAAAGGCTCCATCTCCTGGACCCAT
GTGCATCCTAGGAGACCTAATGTTTCACTACTGAGCCTAAGCCTT
GGGGGAGAGCACCCGGTCAGAGAGATGTGGGTTTGGGGGTCTCTT
CTGCTTCTGCCCCAAGCCACAGCTTTAGATGAAGGCACCTATTAT
TGTCTCCGAGGAAACCTGACCATCGAGAGGCACGTGAAGGTCATT
GCAAGGTCAGCAGTGTGGCTCTGGCTGTTGAGAACTGGTGGATGG
ATAGTCCCAGTGGTGACTTTAGTATATGTCATCTTCTGTATGGTT
TCTCTGGTGGCTTTTCTCTATTGTCAAAGA
CD19 antigen amino acid sequence
(SEQ ID NO: 76)
RPQKSLLVEVEEGGNVVLPCLPDSSPVSSEKLAWYRGNQSTPFLE
LSPGSPGLGLHVGSLGILLVIVNVSDHMGGFYLCQKRPPFKDIWQ
PAWTVNVEDSGEMFRWNASDVRDLDCDLRNRSSGSHRSTSGSQLY
VWAKDHPKVWGTKPVCAPRGSSLNQSLINQDLTVAPGSTLWLSCG
VPPVPVAKGSISWTHVHPRRPNVSLLSLSLGGEHPVREMWVWGSL
LLLPQATALDEGTYYCLRGNLTIERHVKVIARSAVWLWLLRTGGW
IVPVVTLVYVIFCMVSLVAFLYCQR

In certain embodiments, the method further comprises administering an anti-PD1 immunotherapy to the subject. In certain embodiments, the anti-PD1 immunotherapy is an anti-PD1 antibody, such as a monoclonal anti-PD1 antibody, examples of which include but are not limited to, Pembrolizumab, Nivolumab, Cemiplimab, and Dostarlimab. The anti-PD1 immunotherapy is administered prior to, concurrently with, or after the administration of the population of modified cells.

Methods for administration of immune cells for adoptive cell therapy are known and may be used in connection with the provided methods and compositions. For example, adoptive immune cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338; Lee et al., Int J. Mol Sci. (2021) 22(9):4590; Banerjee et al., JCO Clin Cancer Inform. (2021) 5:668-678; Robbins et al., Stem Cell Res Ther. (2021) 12(1):350; Wrona et al., Int J Mol Sci. (2021) 22(11):5899; Atrash and Moyo, Onco Targets Ther. (2021) 14:2185-2201; Martinez Bedoya et al., Front Immunol. (2021) 12:640082; Morgan et al., Front Immunol. (2020) 11:1965; Chicaybam et al., Cancers (Basel) (2020) 12(9):2360; and Rafiq et al., Nat Rev Clin Oncol. (2020) 17(3):147-167. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cell of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include certain leukemia or lymphoid malignancies, benign and malignant tumors, and malignancies e.g., sarcomas, carcinomas, and melanomas. Exemplary cancers include but are not limited to B-cell malignancies such as B-cell lymphomas and leukemias and the like, as well as colorectal cancer, breast cancer, ovarian cancer, renal cancer, non-small cell lung cancer, melanoma, lymphoma, and hepatocellular cancers. The cancers may be non-solid tumors (such as hematological tumors) or solid tumors. Adult tumors/cancers and pediatric tumors/cancers are also included. In one embodiment, the cancer is a solid tumor or a hematological tumor. In certain embodiments, the cancer is a leukemia and/or a lymphoma. In certain embodiments, the cancer cells express CD19.

The cells to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, alymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MED14736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009); Herman et al. J. Immunological Methods, 285(1): 25-40 (2004); Kiesgen et al., Nat Protoc. (2021) 16(3):1331-1342; and Maldini et al., J Immunol Methods (2020) 484-485:112830. In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol BloodMarrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In one aspect, the invention includes a method of treating cancer in a subject in need thereof, comprising administering to the subject any one of the modified immune or precursor cells disclosed herein. Yet another aspect of the invention includes a method of treating cancer in a subject in need thereof, comprising administering to the subject a modified immune or precursor cell generated by any one of the methods disclosed herein.

### F. Sources of Immune Cells

In certain embodiments, a source of immune cells (e.g. T cells) is obtained from a subject for ex vivo manipulation and/or in vivo transduction. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human. Methods for in vivo transduction of immune cells for CAR expression are described, e.g., in Pfeiffer et al., EMBO Mol Med. (2018) 10(11):e9158; Weidner et al., Nat Protoc. (2021) 16(7):3210-3240; Frank et al., Blood Advances (2020) 4(22):5702-5715; Nawaz et al., Blood Cancer J. (2021) 11(6):119.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a macrophage, or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell, or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. n yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### G. Expansion of Immune Cells

Whether prior to or after modification of cells to express a CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### H. Pharmaceutical Compositions and Formulations

Also provided are populations of immune cells of the invention, compositions containing such cells and/or enriched for such cells, such as in which cells expressing CAR make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Mice

Mice were housed in AAALAC-accredited animal facilities at University of Pennsylvania (Upenn) under specific-pathogen-free conditions. Protocols were approved by Upenn IACUC and studies complied with ethical regulations and humane endpoints. Male C57BL/6NTac aged 8 weeks were purchased from Taconic Farms, 8-12 weeks old male NOD.Cg-Prkdc^{scid}Il2rg^{tm1Wjl} (NSG) mice were supplied by Upenn Stem Cell and Xenograft Core.

### Tumor Line and Treatment

The human Acute lymphoblastic leukemia cell line NALM6 and Pancreatic Adenocarcinona AsPC1 were obtained from the American Type Culture Collection and authenticated by the University of Arizona Genetics Core. The murine pancreatic PDA7940b cell line was established from the Kras^{LSL.G12D/+}p53^{R172H/+} (KPC) mouse pancreatic tumor model, was provided by Gregory Beatty (Upenn).

NALM6 tumor cells were irradiated by X-ray for 10,000 cGy. 1e7 irradiated NALM6 (IR NALM6) cells were intravenously injected into NSG mice every 4 days for 6 doses in total. C57BL/6 mice were injected subcutaneously on the back with 1e6 PDA7940b tumor cells in 100 μL PBS, NSG mice were injected subcutaneously on the back with 1e6 AsPC1 tumor cells in 100 μl 1× Matrigel. Tumor growth was monitored weekly. Tumor volume was determined as length (mm)×width (mm)×0.5. C57BL/6 tumor-bearing mice were infused with 1e6 mouse CAR-T cells 7 days after tumor inoculation. PDA7940b bearing C57BL/6 mice were treated with 1e9 pfu Ad-MCV-mCD19t-eGFP intratumorally and 200 μg αPD-1 (clone 29 F.1A12) monoclonal antibody intraperitoneally with in 200 μl PBS every other day from day 5 post CAR-T infusion. NSG tumor-bearing mice were infused with 3e5 or 1e5 CAR-T cells on 21 days post tumor inoculation. 1e7 IR NALM6 cells were infused every 4 days from day −1 post CAR-T infusion.

### Flow Cytometry

Single-cell suspensions were prepared from livers, tumors, spleens. Cells were incubated with Fc block (anti-CD16/32 clone 93, BioLegend, or Human TruStain FcX™) and stained with the fluorochrome-conjugated monoclonal antibodies listed below. FMOs were used as negative staining controls to set gates. Zombie NIR Fixable Viability Kit and the following fluorochrome-conjugated monoclonal antibodies were purchased from BioLegend: 1) antibodies for human: BV605-anti-CD45 (2D1), BV421-anti-PD1 (EH12.2H7), BV510-anti-CD8 (HITa), BV11-anti-CD4 (RPA-T4), BV785-anti-CD3 (OKT3), PE-Cy7-anti-CD27 (M-T271), APC-Cy7-anti-CD39 (A1), Alexa Fluor 488-anti-CD45RA (H1100), 2) antibodies for mouse: Alexa Fluor 700-anti-CD45 (30-F11), BV421-anti-PD1 (29F. 1A12, RMP1-30), PE-Cy7-anti-CD44 (TM7), BV510-anti-CD45.1 (A20), BV711-anti-CD4 (RM4-5), BV785-anti-CD19 (6D5), BV605-anti-CD8a (53-6.7), PerCP/Cy5.5-anti-Tim3 (B8.2C12), Alexa Fluor 647-anti-CD39 (Duha59), FITC-anti-CD62L (MEL-14), PE/Dazzle M 594-anti-Lag3 (C9B7W); from Thermofisher: antibodies for human: PE-eFluor 610-anti-Lag3; and from BD Biosciences: antibodies for human: Alexa Fluor 700-anti-CD197 (150503). Flow cytometry data were acquired on an LSR Fortessa flow cytometer (BD Biosciences) and analyzed using FlowJo software (Tree Star).

The results of the experiments are now described:

### Example 1: Dual CAR Design and Expression

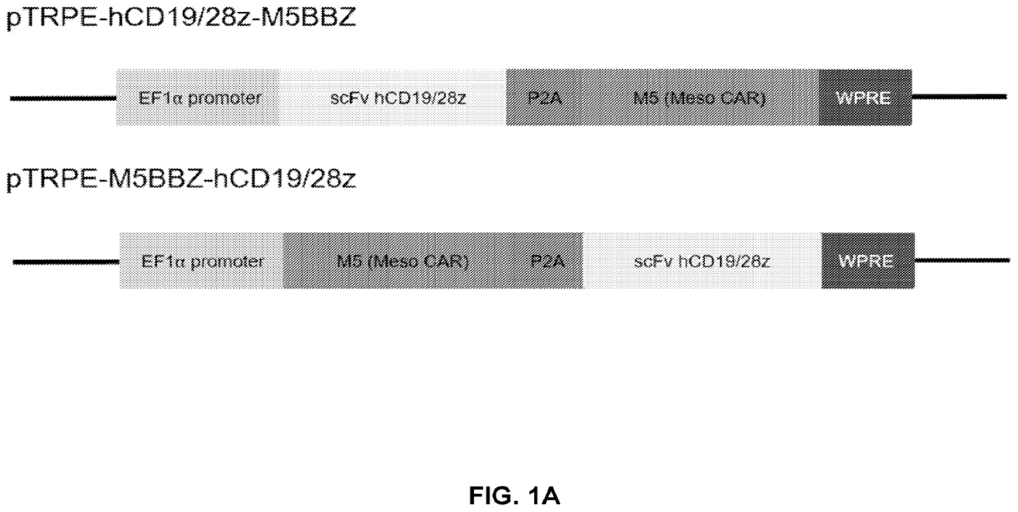

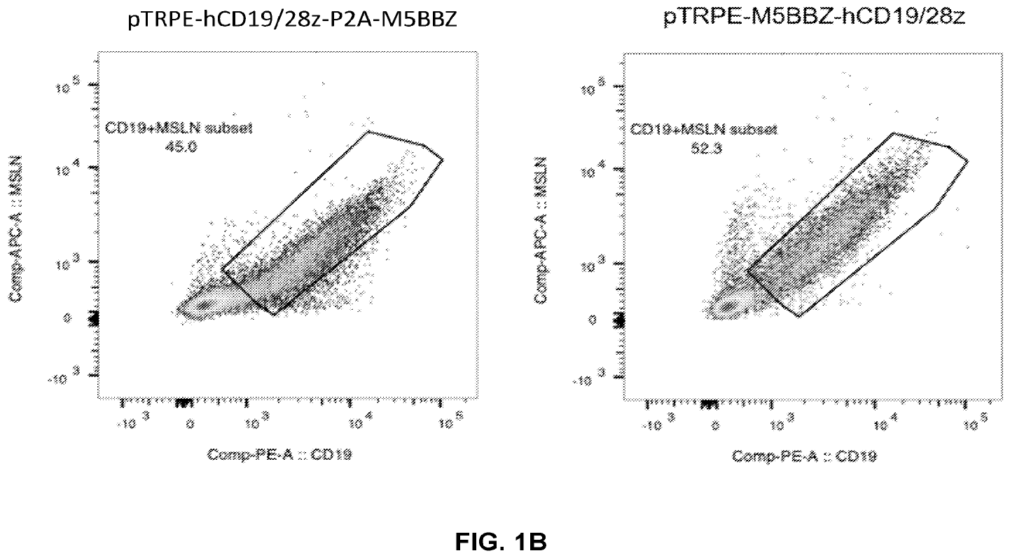

Two dual CAR constructs were designed as shown in FIG. 1A and each cloned into a pTRPE lentiviral vector. The “pTRPE-hCD19/28z-M5BBZ” dual CAR construct encodes a first CAR comprising an anti-human CD19 scFv (“hCD19) and an intracellular domain comprising a human CD28 costimulatory domain (“28”) and a human CD3 zeta signaling domain (“z”), followed by a P2A linker, followed by a second CAR comprising an anti-human mesothelin (MSLN) scFv (“M5”) and an intracellular domain comprising a human 4-1BB costimulatory domain and a human CD3 zeta signaling domain (“BBZ”), followed by a Woodchuck Hepatitis Virus (WHP) Posttranscriptional Regulatory Element (WPRE). As such, the pTRPE-hCD19/28z-M5BBZ dual CAR construct encodes the hCD19/28z CAR upstream (i.e., 5′) from the M5BBZ CAR. The “pTRPE-M5BBZ-hCD19/28z” dual CAR construct encodes the M5BBZ CAR upstream (i.e., 5′) from the hCD19/28z CAR with a P2A linker between the two CARs and WPRE after the hCD19/28z CAR. In both dual CAR constructs, expression is controlled by a constitutive EF1α promoter.

Additional constructs encoding a single CAR, used as controls in the experiments described herein, included a pNVS lentiviral construct encoding a single CAR comprising anti-human CD19 scFv, a human 4-1BB costimulatory domain and a human CD3 zeta signaling domain (“pNVS-hCD19BBZ”), and a pTRPE lentiviral construct encoding a single CAR comprising an anti-human mesothelin (MSLN) scFv (“M5”) and an intracellular domain comprising a human 4-1BB costimulatory domain and a human CD3 zeta signaling domain (“pTRPE-M5BBZ”).

The two dual CAR constructs were each transduced into T cells and expression of the hCD19/28z CAR and the M5BBZ CAR was assessed via flow cytometry. As shown in FIG. 1B, T cells comprising either dual CAR construct expressed both CARs, although the pTRPE-hCD19/28z-M5BBZ dual CAR construct provided higher expression of both CARs compared to the pTRPE-M5BBZ-hCD19/28z dual CAR construct.

A murine version of the the pTRPE-hCD19/28z-M5BBZ dual CAR construct was also designed and cloned into a MSGV retroviral vector. This murine dual CAR construct (“MSGV-anti-moCD19-MuCD28z-anti-moMeso-A03-3-MuBBz”) encodes a first CAR comprising an anti-murine CD19 scFv and a murine CD28 costimulatory domain and a murine CD3 zeta signaling domain, and a second CAR comprising an anti-murine mesothelin scFv (“A03”), a murine 4-1BB costimulatory domain, and a murine CD3 zeta signaling domain. Control murine CAR constructs include “MSGV-anti-moCD19-MuBBz” encoding a single CAR comprising an anti-murine CD19 scFv, a murine 4-1BB costimulatory domain, and a murine CD3 zeta signaling domain, and “MSGV-anti-moMeso-A03-3-MuBBz” encoding a single CAR comprising an anti-murine mesothelin scFv (“A03”), a murine 4-1BB costimulatory domain, and a murine CD3 zeta signaling domain.

### Example 2: Dual CAR T Cell Expansion In Vitro

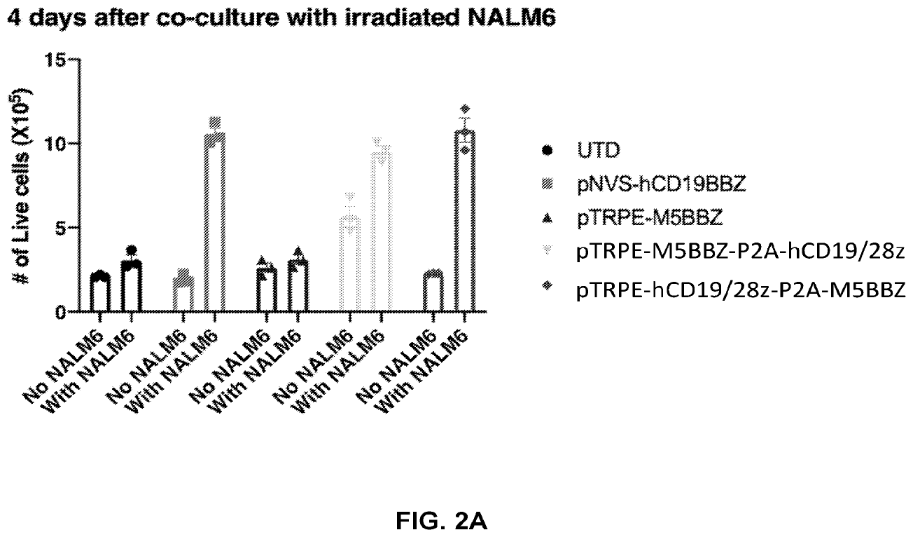

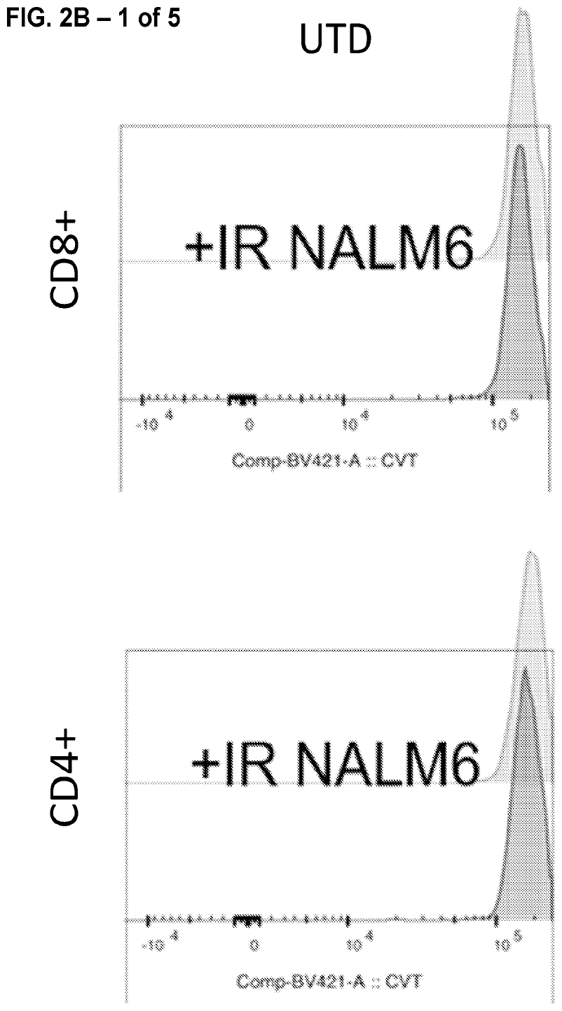

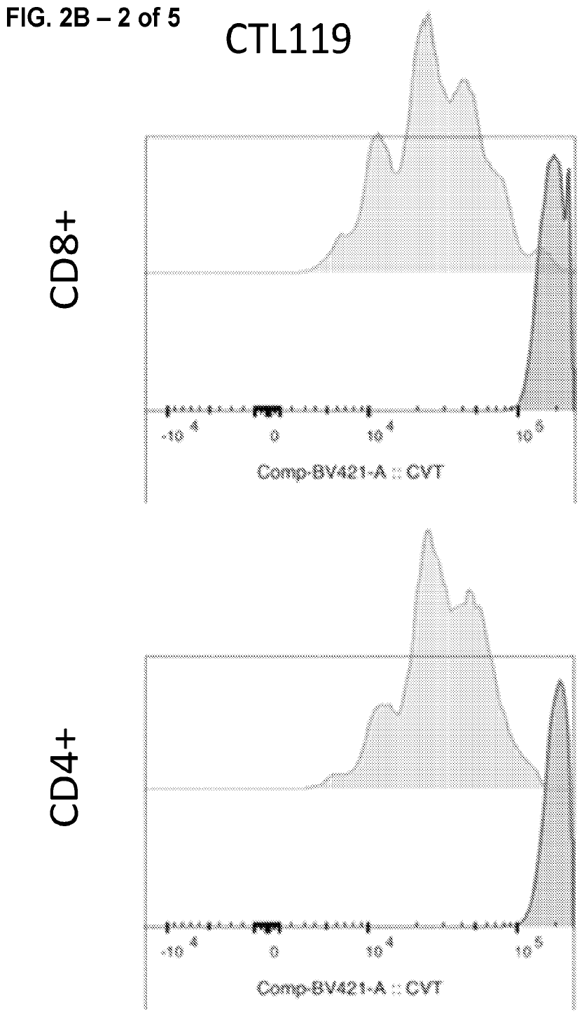

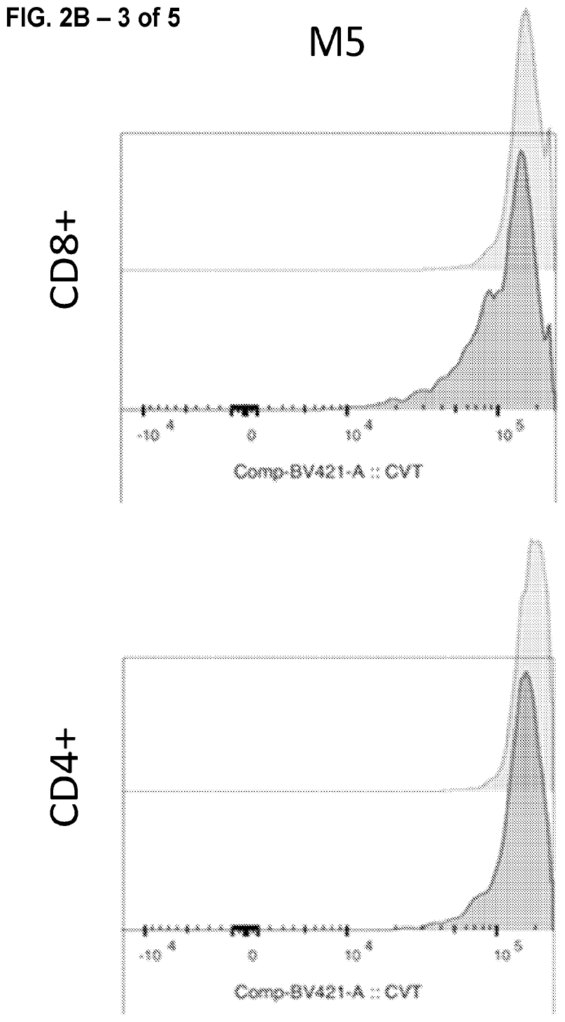

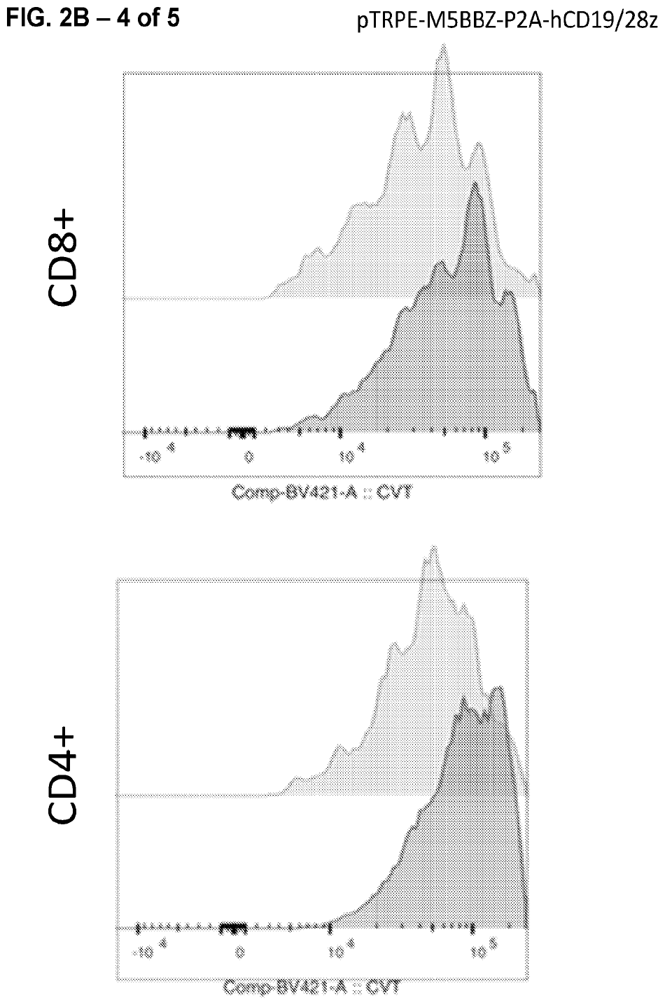

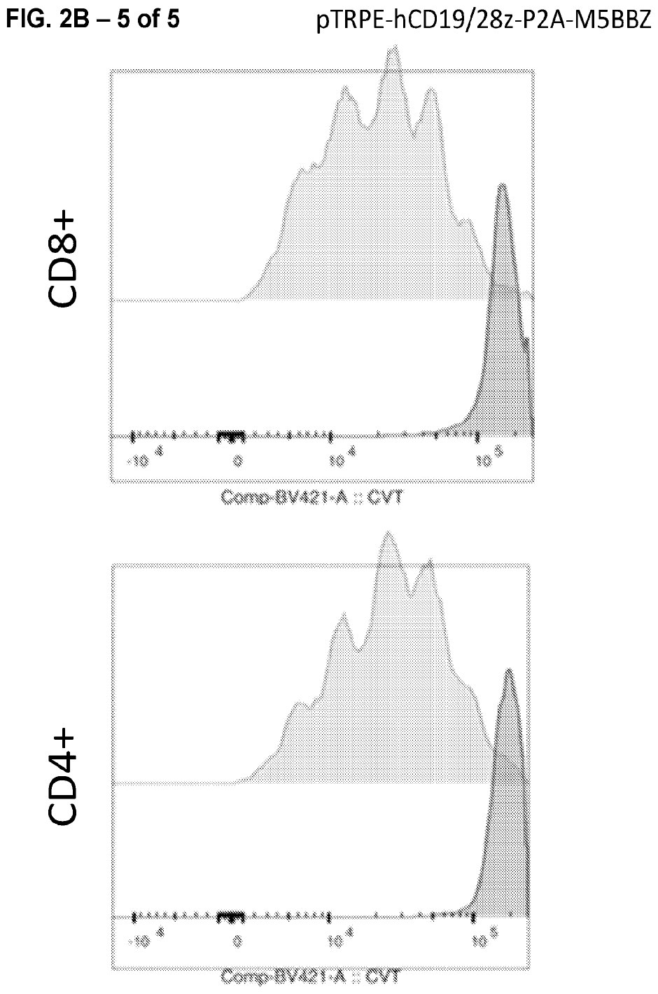

T cells were transduced with either dual CAR construct (pTRPE-hCD19/28z-M5BBZ or pTRPE-M5BBZ-hCD19/28z), or with pNVS-hCD19BBZ or pTRPE-M5BBZ single CAR constructs as controls. The T cells were co-cultured in vitro with and without irradiated Nalm6 human tumor cell line for 4 days. Irradiated Nalm6, which expresses human CD19, simulate endogenous CD19+ B cells. Both dual CAR constructs and pNVS-hCD19BBZ exhibited increased cell numbers of CAR T cells when “boosted” with irradiated NALM6 cells (FIG. 2A). However, the pTRPE-hCD19/28z-P2A-M5BBZ dual CAR showed less tonic signaling (i.e., less ligand-independent constitutive signaling) without this CD19 antigen boost. As expected, numbers of pTRPE-M5BBz CAR T cells were not boosted upon co-culture with the NALM6 cells (FIG. 2A).

To further evaluate CAR T cell expansion, the transduced CAR T cells were labelled with cell trace violet (CTV) and co-cultured with irradiated Nalm6 for 4 days. CD8+ and CD4+ T cells were analyzed for proliferation by means of division peak. CSFE staining illustrated similar CAR T expansion for CD4+ and CD8+ T cells transduced with pNVS-hCD19BBZ single CAR construct, pTRPE-M5BBZ-hCD19/28z dual CAR construct, or pTRPE-hCD19/28z-M5BBz dual CAR construct, but not for pTRPE-M5BBz CAR T cells (FIG. 2B).

### Example 3: Boosting Dual CAR T Cells with CD19 Antigen Accelerates Tumor Cell Killing In Vitro

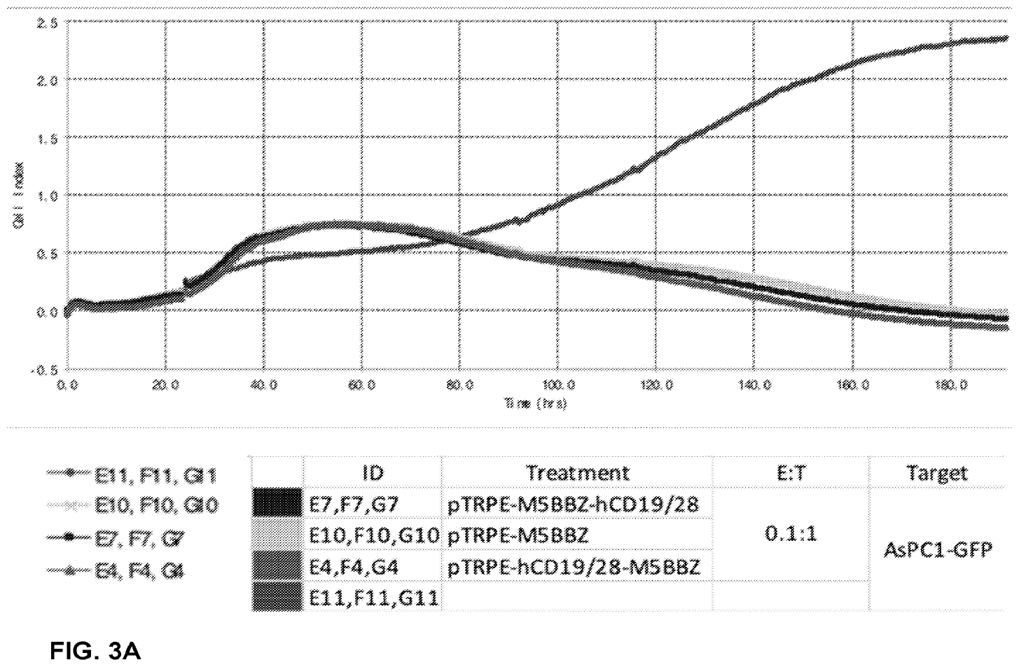

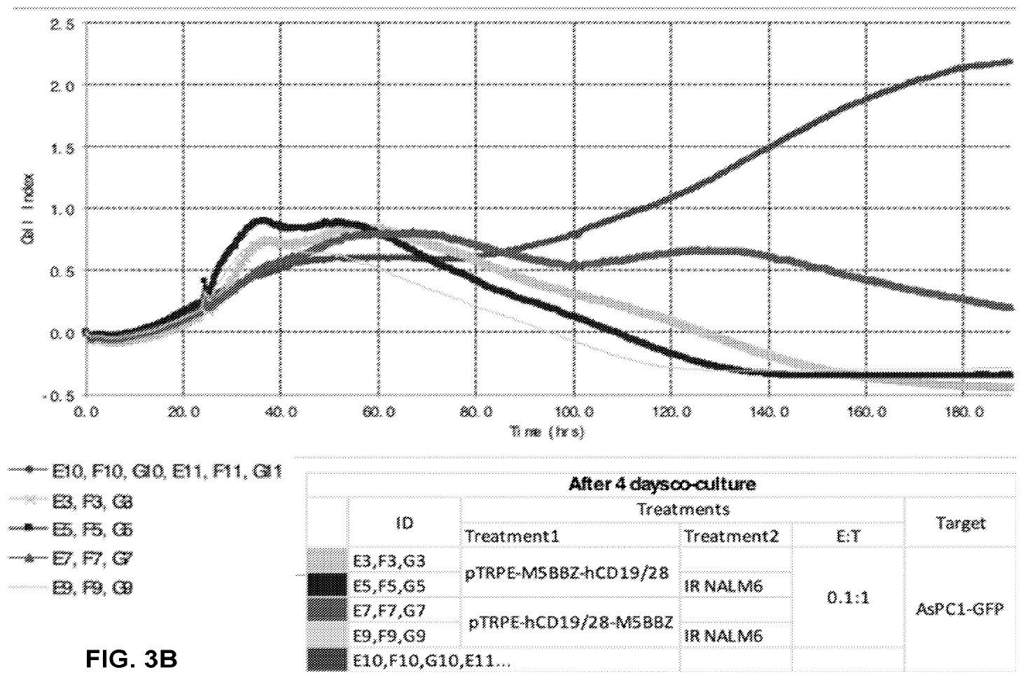

CAR T cell killing of mesothelin-positive AsPC-1 tumor cells in vitro was measured using an xCelligence impedance assay. Cells were seeded at 0.1:1 effector to target ratio (E:T ratio) before and after co-culture with irradiated CD19+ NALM6 tumor cells (which simulate endogenous CD19+ B cells). Before boosting with CD19+ irradiated NALM6 tumor cells, T cells transduced with either dual CAR construct or the pTRPE-M5BBZ single CAR construct killed AsPC1-GFP cells equally well in vitro (FIG. 3A). Surprisingly, after co-culture with irradiated CD19+ NALM6 tumor cells, T cells transduced with either dual CAR construct exhibited enhanced and accelerated tumor cell killing (FIG. 3B). Interestingly, the pTRPE-hCD19/28z-M5BBZ dual CAR-T cells showed superior (i.e., accelerated) tumor cell killing compared to pTRPE-M5BBZ-hCD19/28z dual CAR T cells after co-culture with the CD19+ irradiated NALM6 cells (FIG. 3B).

### Example 4: Dual CAR T Cells Exhibit Lower On-Target Off-Tumor Cytotoxicity In Vitro

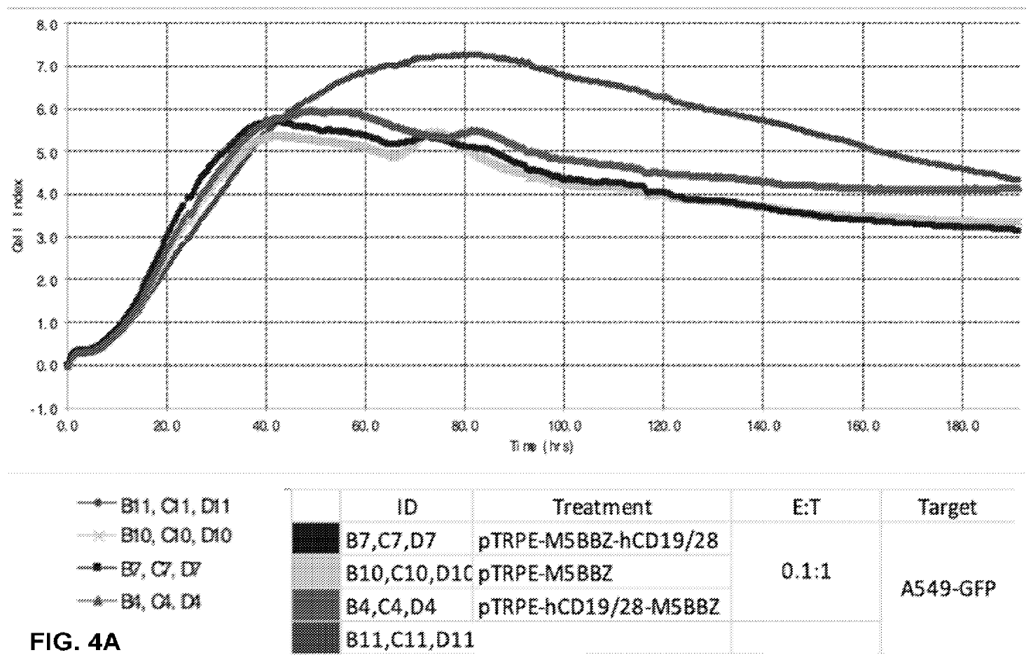

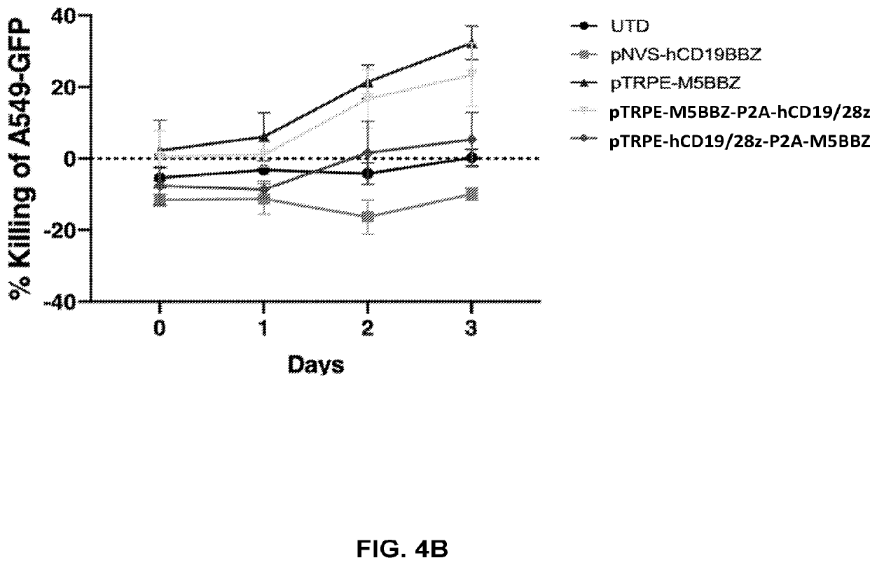

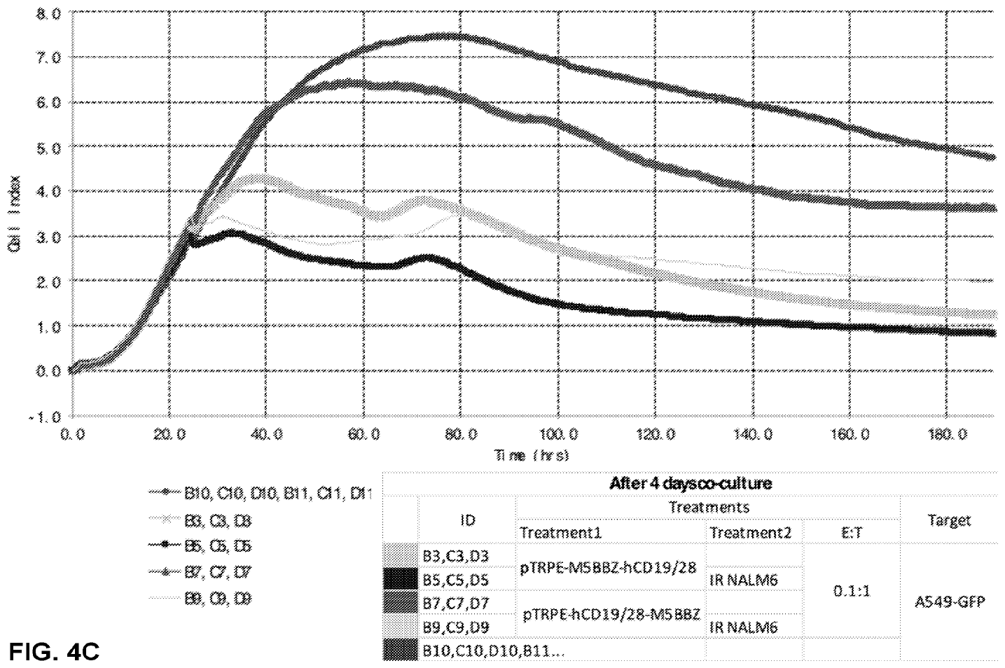

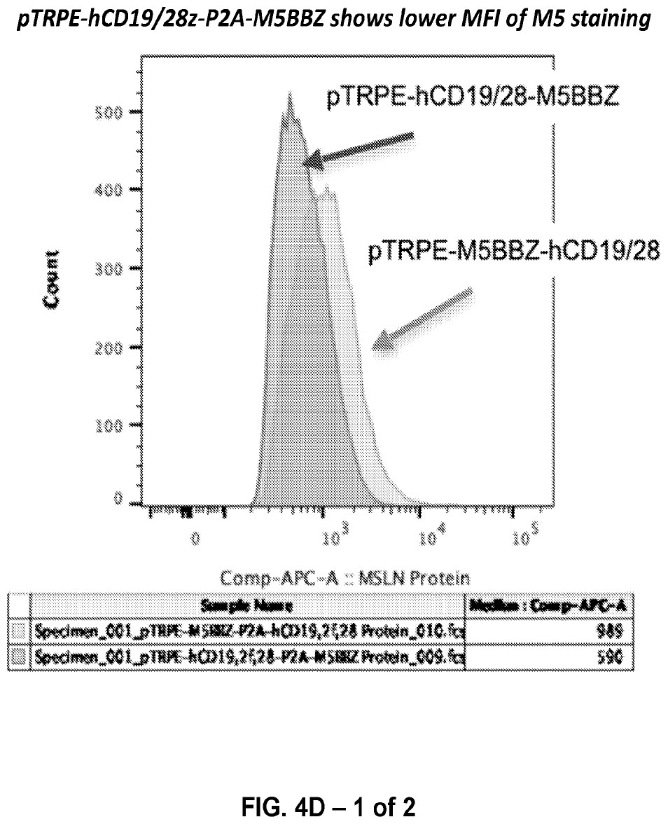

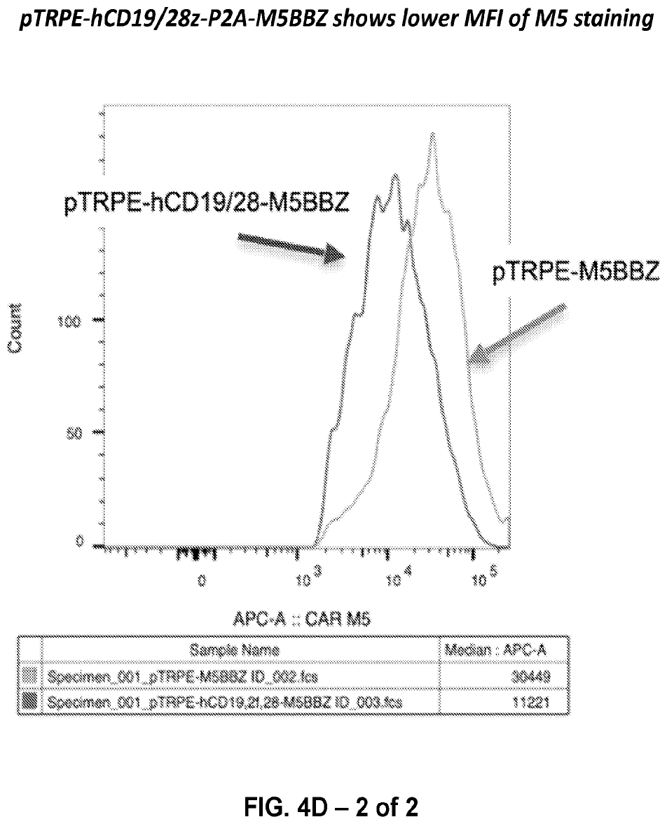

CAR T cell killing of mesothelin-positive A549 cells in vitro was measured using an xCelligence impedance assay as a model to assess potential on-target off-tumor toxicity in lung tissue of T cells transduced with a dual CAR construct. The cytotoxicity of CAR T cells against A549 cells were measured by the percentage of killing of A549-GFP cells over time at E:T=0.1:1. T cells transduced with the pTRPE-hCD19/28z-M5BBZ dual CAR construct showed reduced cytotoxicity of A549 cells compared to T cells transduced with pTRPE-M5BBZ-hCD19/28z dual CAR or pTRPE-M5BBZ single CAR constructs (FIG. 4A and FIG. 4B).

Surprisingly, co-culture of the dual CART cells with CD19+ irradiated Nalm6 cells (which simulate endogenous CD19+ B cells) reduced A549 cell cytotoxity of T cells transduced with either dual CAR construct (FIG. 4C) compared to T cells not co-cultured with irradiated Nalm6. However, T cells transduced with the pTRPE-hCD19/28z-M5BBZ dual CAR construct exhibited a greater reduction in cytotoxicity of A549 cells after coculture with irradiated NALM6 cells compared to T cells transduced with the pTRPE-M5BBZ-CD19/28z dual CAR construct (FIG. 4C). The increased therapeutic index of T cells transduced with the pTRPE-hCD19/28z-M5BBZ dual CAR construct may be because these T cells express lower levels of the M5BBZ CAR compared to T cells transduced with the pTRPE-M5BBZ-hCD19/28z dual CAR construct, as indicated by the lower MFI of M5BBZ CAR staining for these cells (FIG. 4D).

### Example 5: Boosting Dual CAR T Cells with CD19 Antigen Stimulates CAR T Expansion in Tumor-Free NSG Mice

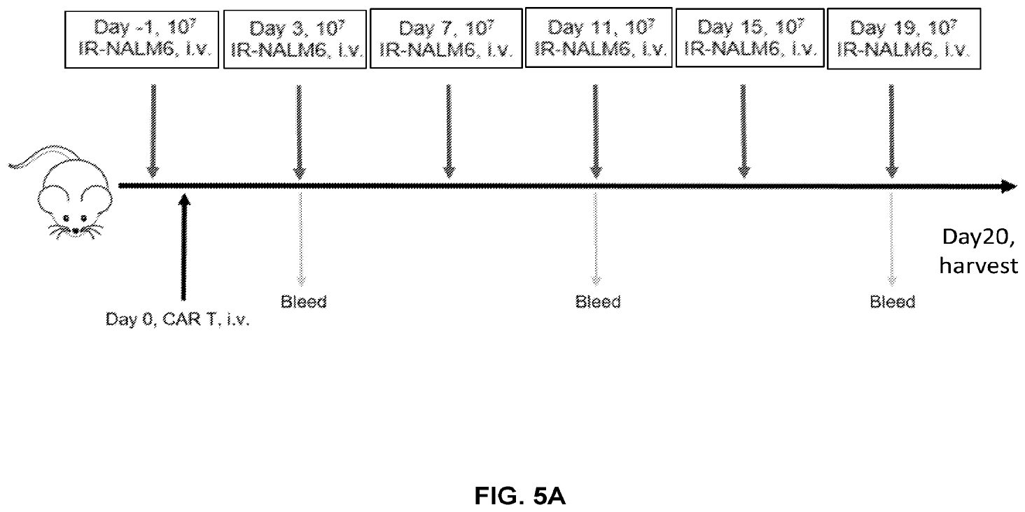

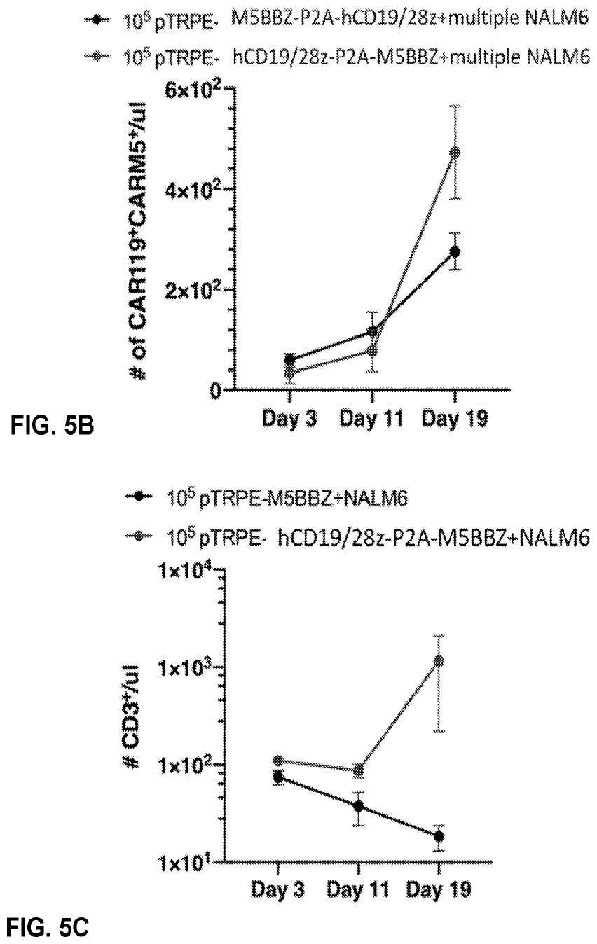

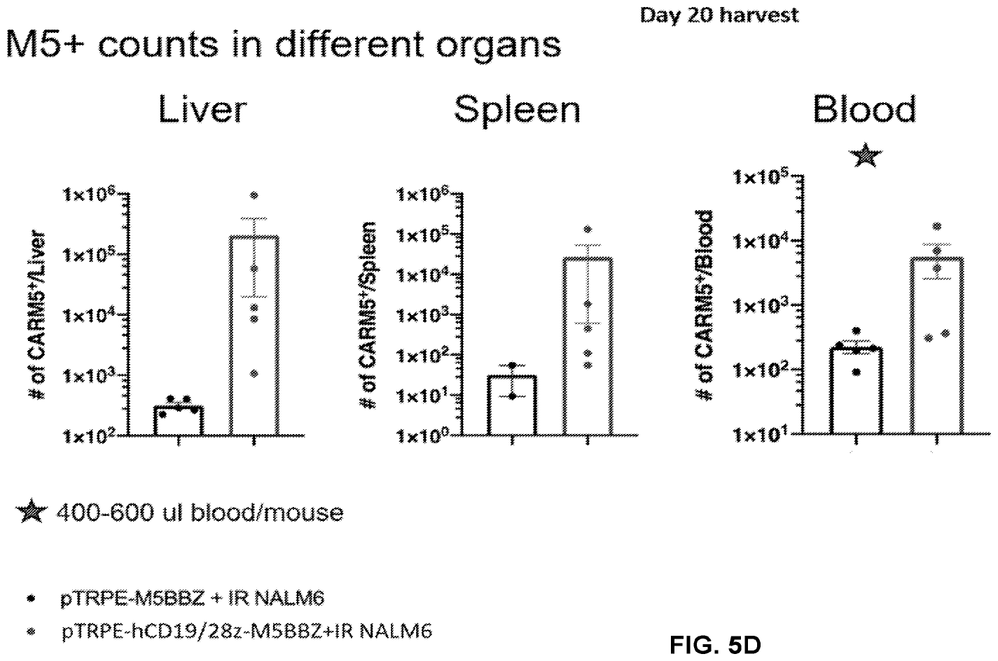

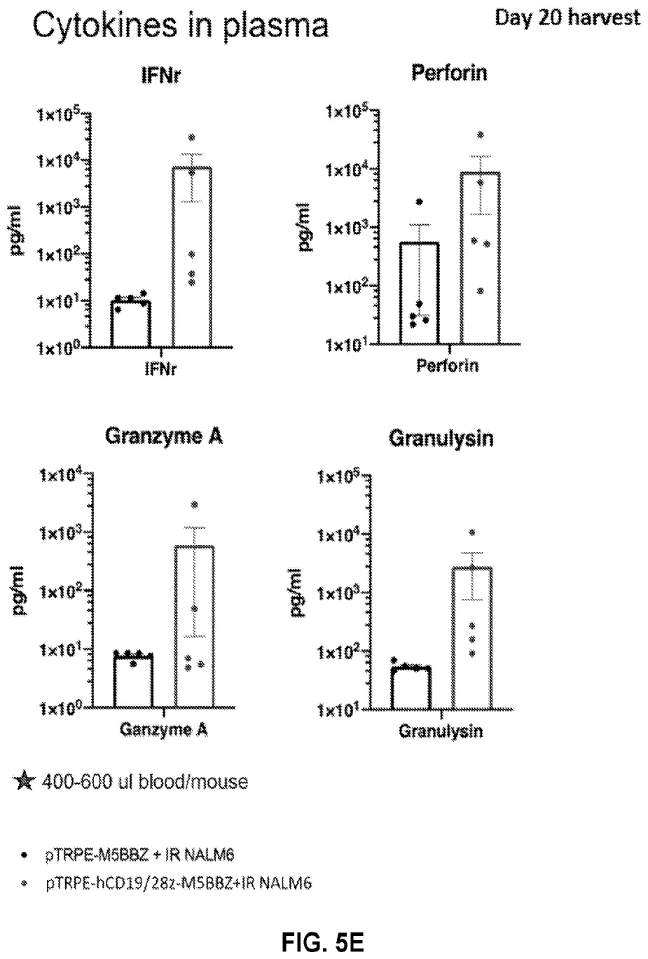

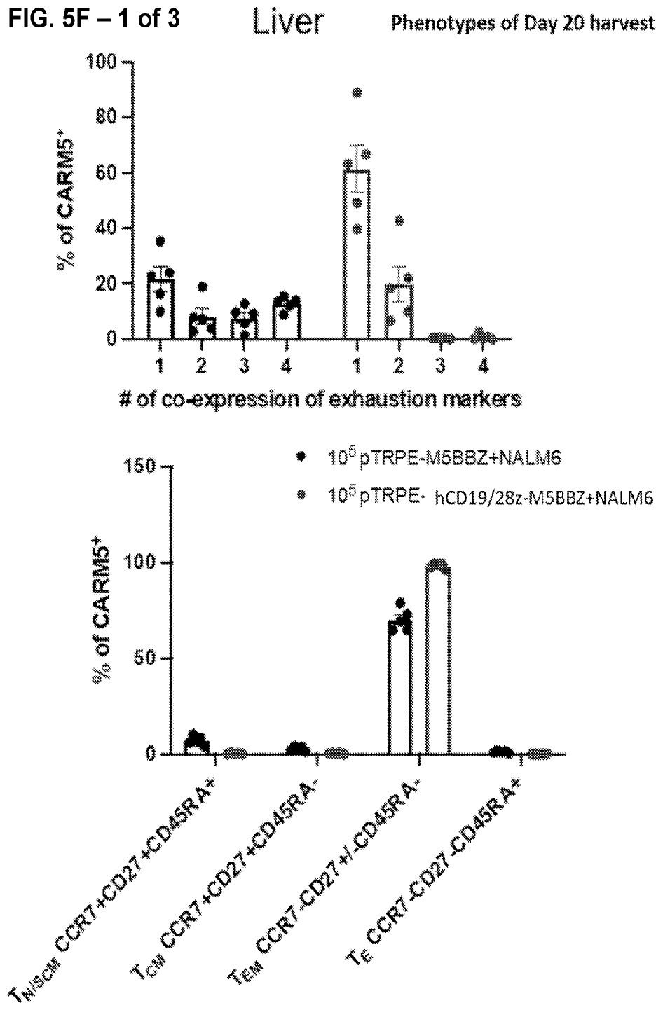

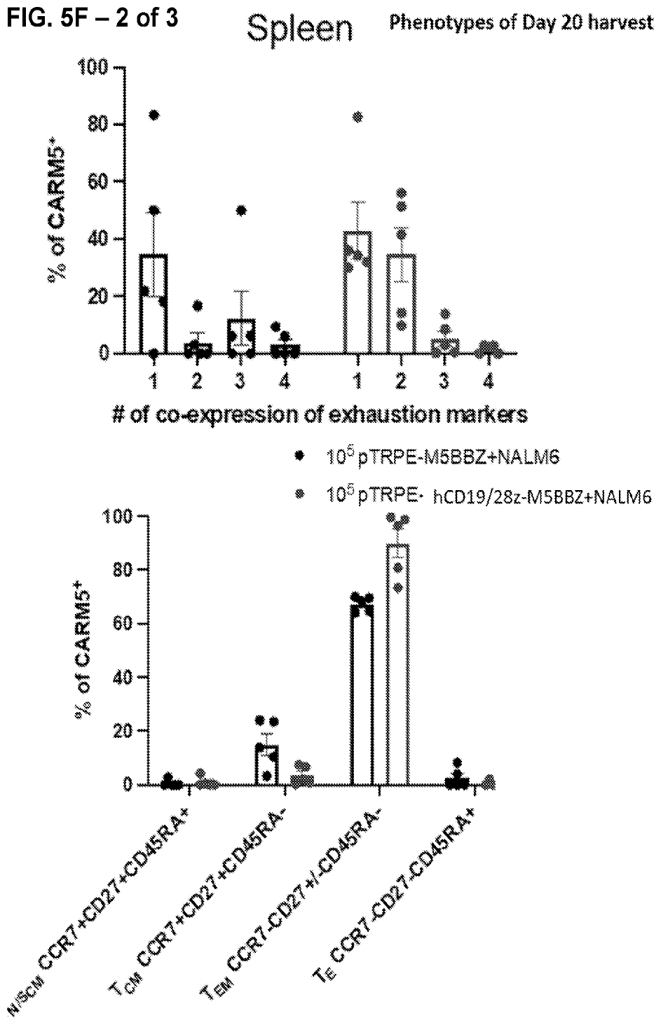

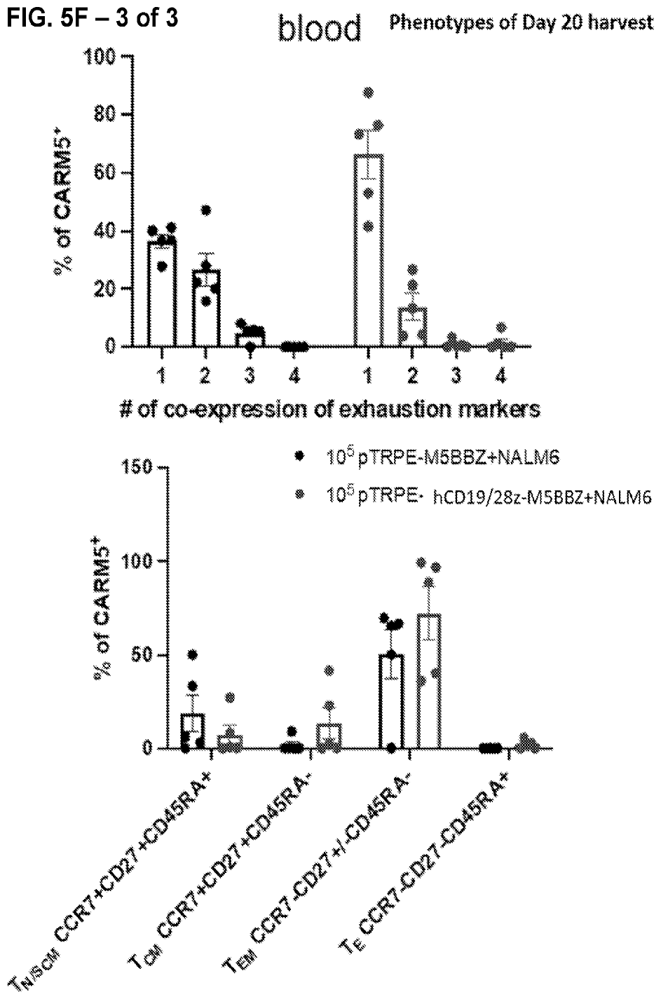

To assess expansion of T cells transduced with dual CAR constructs in tumor-free NSG mice, the mice were first administered 10⁷ irradiated Nalm6 cells (which simulate endogenous CD19+ B cells) on Day −1, followed by administration of 10⁵ CAR T cells on day 0 (FIG. 5A). Administration of irradiated Nalm6 was repeated on day 3, day 7, day 11, day 15, and day 19, the mice were bled on day 3, day 11, and day 19, and cells were harvested on Day 20 (FIG. 5A). CAR T cell expansion was assessed in the blood samples collected on day 3, day 11, and day 19. T cells transduced with either the pTRPE-hCD19/28z-M5BBZ dual CAR construct or the pTRPE-M5BBZ-hCD19/28z dual CAR construct expanded in vivo, where the pTRPE-hCD19/28z-M5BBZ dual CAR construct endowed the T cells with superior proliferative capacity compared to the pTRPE-M5BBZ-hCD19/28z dual CAR construct (FIG. 5B). In contrast, T cells transduced with the pTRPE-M5BBZ did not expand (FIG. 5C), illustrating that the stimulation-triggered expansion (i.e., “boosting”) is CD19 antigen driven.

Next, CAR T cell expansion was assessed in the spleen and liver compared to blood, which showed that the pTRPE-hCD19/28z-M5BBZ dual CAR construct significantly enhanced T cell expansion compared to T cells transduced with the pTRPE-M5BBZ single CAR construct (FIG. 5D). Additionally, a significant increase in the secretion of cytotoxic cytokines (interferon production regulator (IFNr), perform, granzyme A, and granulysin) in the serum was detected for T cells transduced with pTRPE-hCD19/28z-M5BBZ dual CAR construct compared to the pTRPE-M5BBZ single CAR construct (FIG. 5E). Liver is the organ which accumulated the majority of expanded CAR T cells in this mouse model, likely due to the use of irradiated NALM6 tumor line to boost CAR T expansion. Based on these findings, it is expected that, in human patients, dual CAR T cell numbers would be boosted by the patient's own CD19+ B cells and thus will expand in the blood and induce B-cell aplasia, as has been previously observed in anti-CD19 CAR T cell therapy in human patients.

The phenotypes of cells harvested at Day 20 were also assessed (FIG. 5F). Importantly, the continuous stimulation of T cells transduced with pTRPE-hCD19/28z-M5BBZ by CD19+ irradiated Nalm6 did not result in an exhausted and terminal differentiated phenotype. The majority of pTRPE-hCD19/28z-M5BBZ CAR T cells still maintained effector memory cell phenotype, which is similar to the T cells transduced with the pTRPE-M5BBZ single CAR construct. Further, most of the T cells transduced with pTRPE-hCD19/28z-M5BBZ dual CAR construct express only one or two exhaustion markers (PD-1 and/or CD39, but not Tim3 or Lag3). Thus, the observed T cell expansion can be considered as activation but not exhaustion.

### Example 6: Dual CAR T Cells Enhances Tumor Cell Killing In Vivo

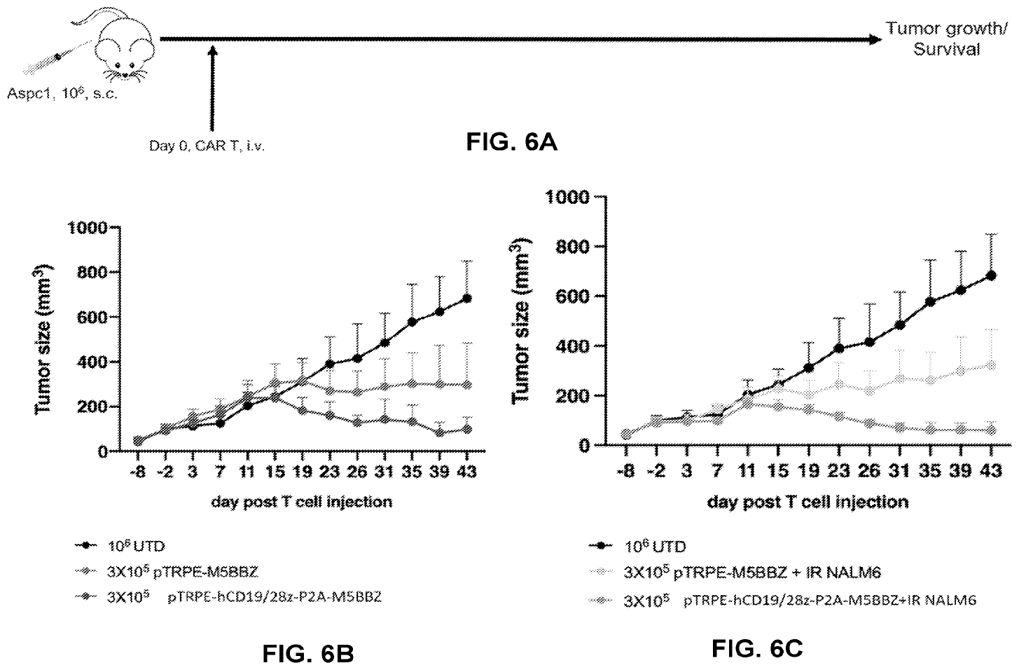

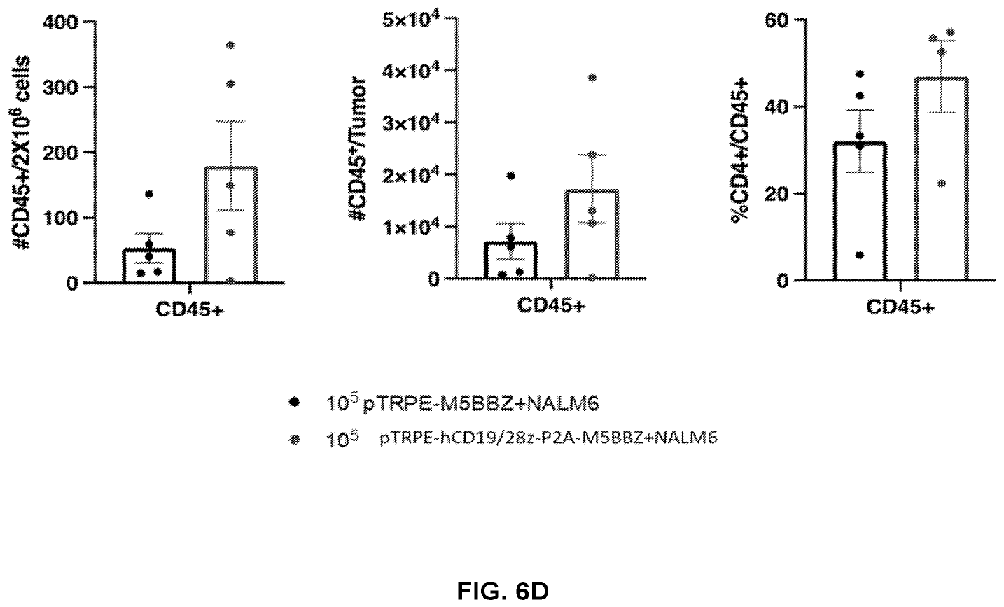

To assess tumor cytotoxicity of T cells transduced with dual CAR constructs in vivo, NSG mice were first administered 10⁶ AsPC1 pancreatic tumor cells to generate the AsPC1 tumor mouse model, followed by administration of 3×10⁵ CAR T cells on day 0 (FIG. 6A). Tumor size was assessed periodically for 43 days post T cell injection (FIG. 6B and FIG. 6C). Even without administration of irradiated Nalm6 to the mice, T cells transduced with the pTRPE-hCD19/28z-M5BBZ dual CAR construct displayed significantly enhanced pancreatic tumor killing in vivo compared to T cells transduced with the pTRPE-M5BBZ single CAR construct (FIG. 6B). Administration of irradiated Nalm6 (which simulate endogenous CD19+ B cells) further enhanced and accelerated the ability of the T cells transduced with the pTRPE-hCD19/28z-M5BBZ dual CAR to kill pancreatic tumor cells in vivo (FIG. 6C). The enhanced anti-tumor effects of T cells transduced with the pTRPE-hCD19/28z-M5BBZ dual CAR construct compared to T cells transduced with the pTRPE-M5BBZ single CAR construct in the AsPC1 tumor mouse model is correlated with higher CAR T cell number in the tumors and a shift of the CAR T cells to CD4+ T cells for the T cells transduced with the pTRPE-hCD19/28z-M5BBZ dual CAR construct (FIG. 6D). These data demonstrate that the CD19 antigen boosts pTRPE-hCD19/28z-M5BBZ dual CAR T cells and promotes the anti-tumor effects of these cells.

### Example 7: Murine Dual CAR T Cells are Similar to the Human Dual CAR T Cells

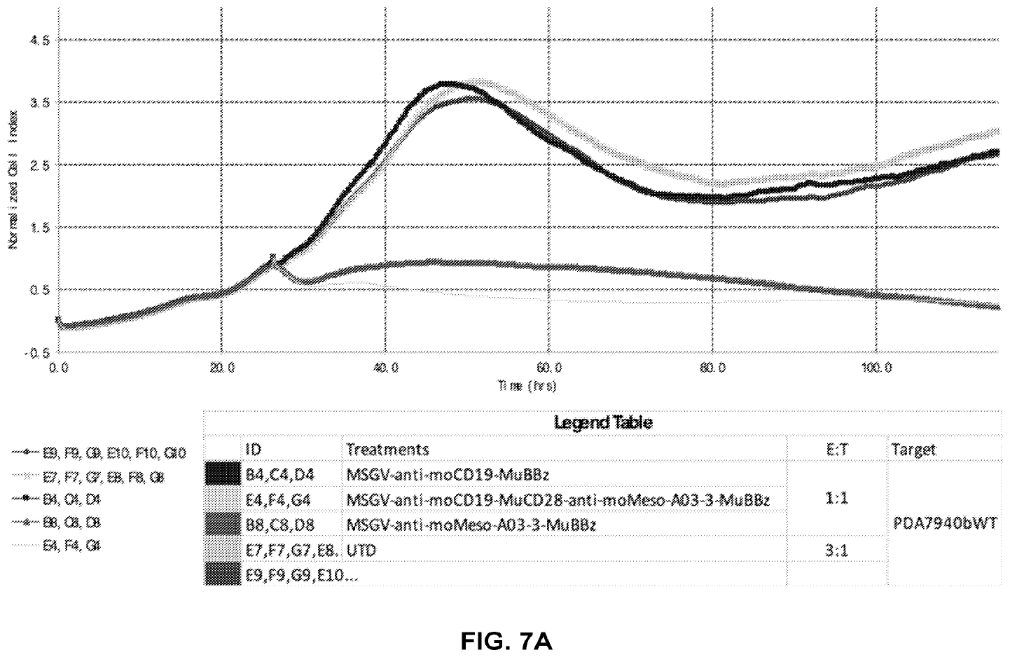

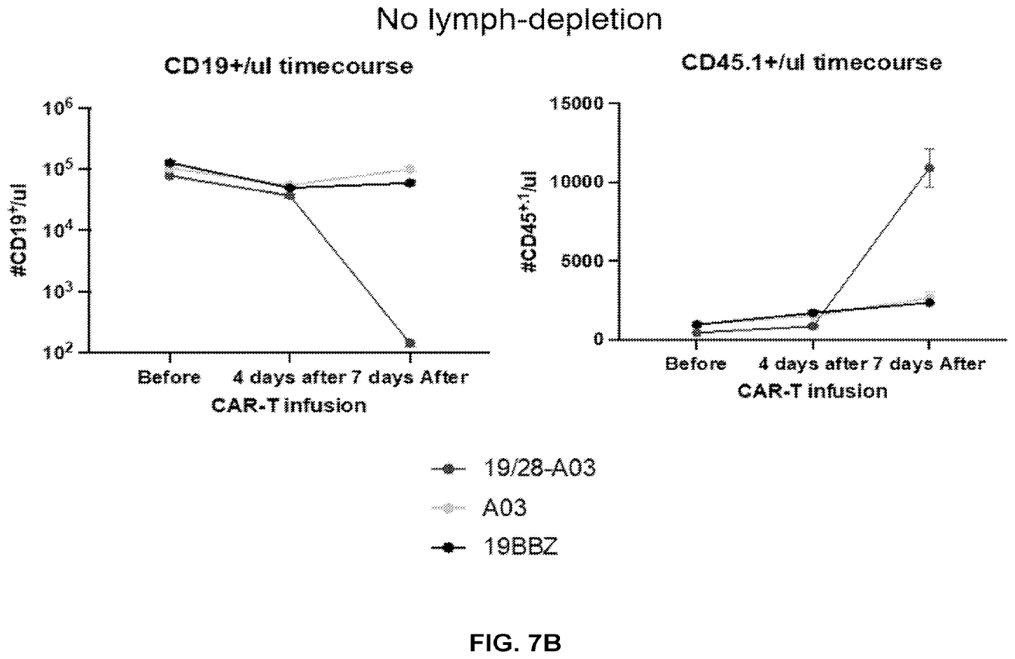

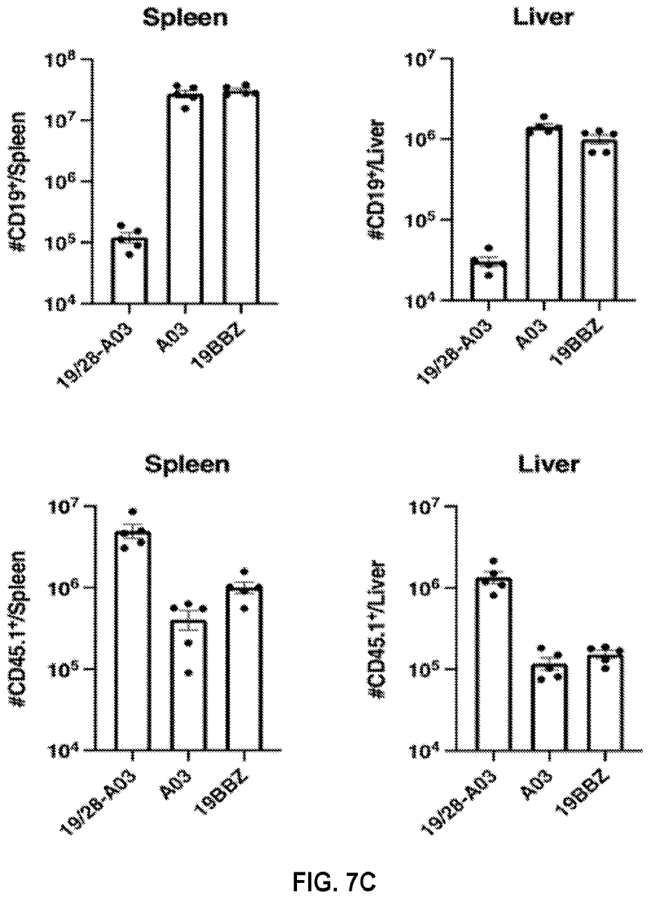

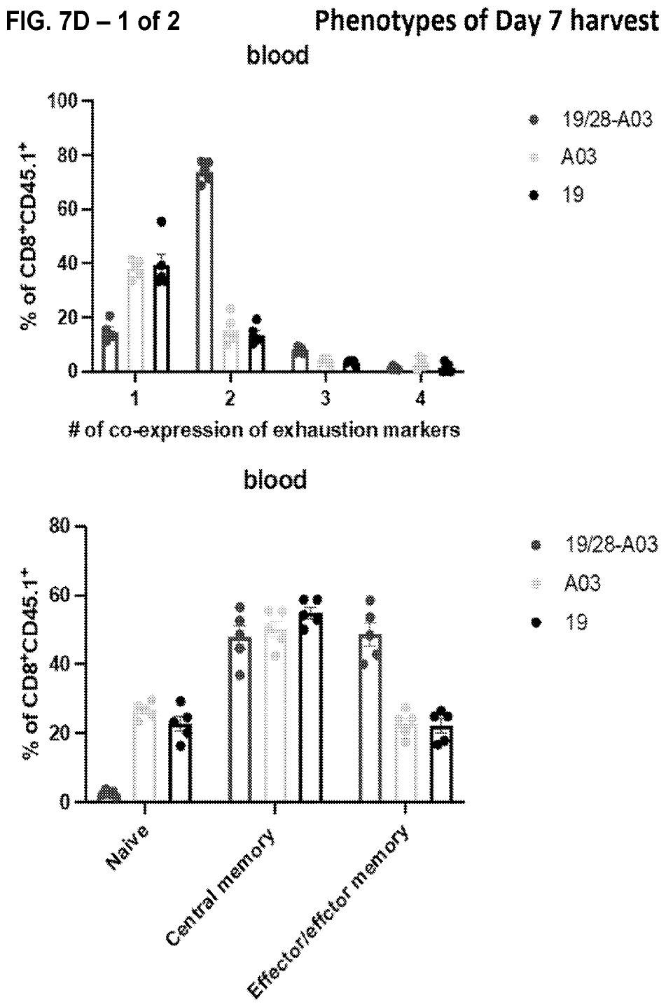

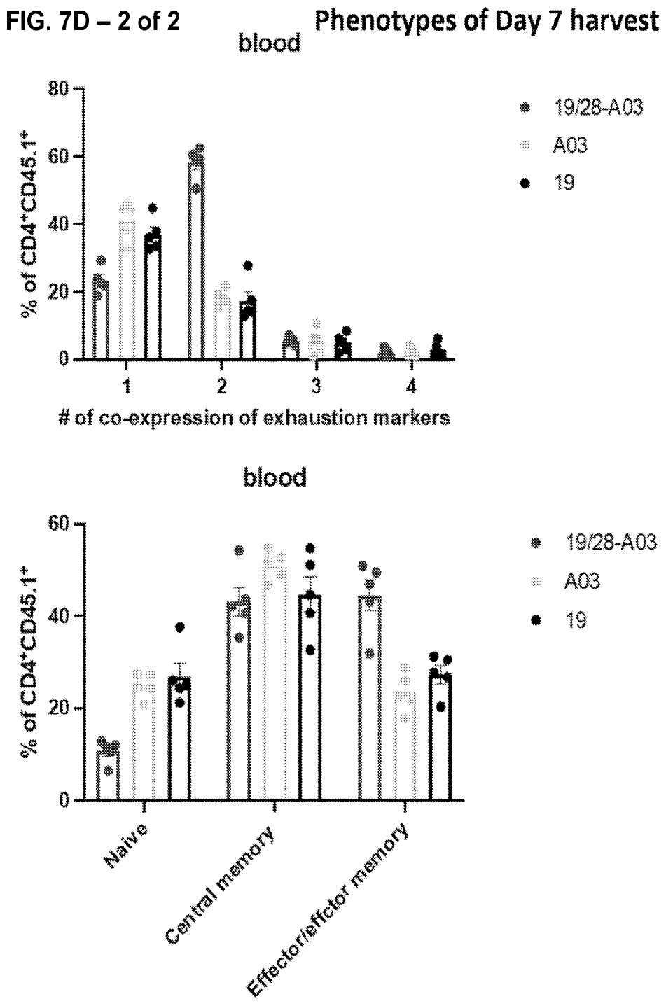

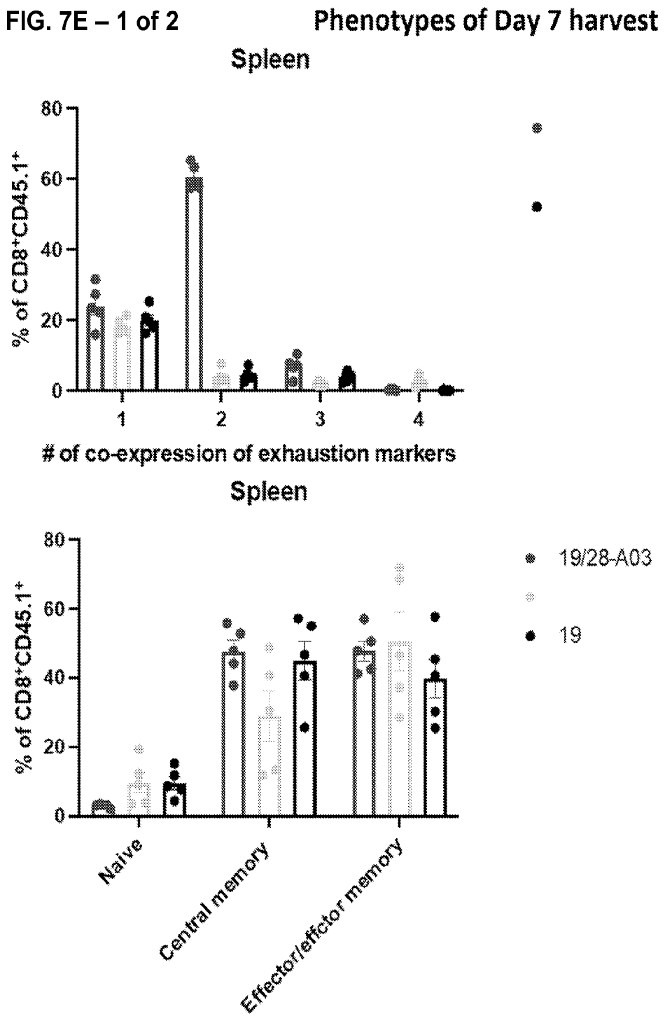

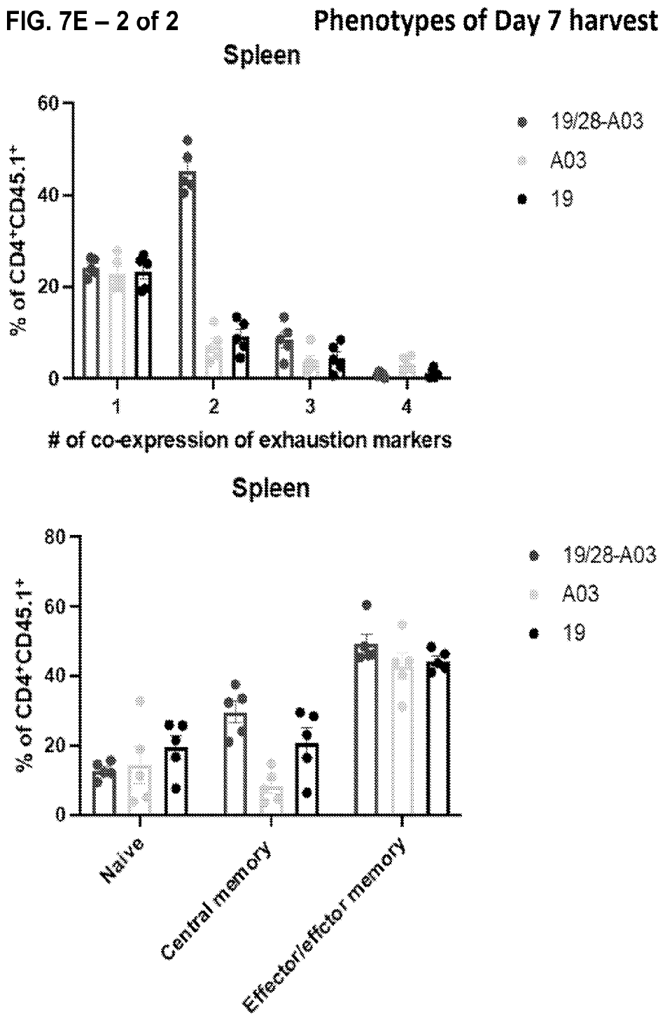

CAR T cell killing of mesothelin-positive PDA7940bWT cell line in vitro was measured using an xCelligence impedance assay. Murine T cells were transduced with the murine dual CAR construct (MSGV-anti-moCD19-MuCD28z-anti-moMeso-A03-3-MuBBz) or murine anti-CD19 single CAR construct or murine anti-MSLN single CAR construct as controls. T cells transduced with the murine dual CAR construct showed enhanced killing of the MSLN+ PDA7940bWT cell compared to T cells transduced with the anti-MSLN single CAR construct (FIG. 7A).

Next, the murine dual CAR T cells were assessed in a tumor-free syngeneic mouse model. Murine T cells transduced with the murine dual CAR construct, but not either single murine CAR construct, were able to engraft/expand in tumor-free syngeneic mice with the depletion of CD19 positive B cells, without lymphodepletion (FIG. 7B). The expanded dual CAR T cells were mainly in spleen compared to liver (FIG. 7C). Phenotypes of T cells in the blood (FIG. 7D) or spleen (FIG. 7E) harvested on Day 7 were analyzed and were similar to each other. The continuous stimulation of dual CAR T cells by the endogenous CD19+B cells did not result in an exhausted and terminal differentiated phenotype. Around 40-50% of murine dual CAR-T cells were phenotypically central memory cells, which was similar to both of the two murine single CAR-T cells. Overall CAR T cells in the syngeneic model had more central memory cells than the CAR T cell in the NSG model with irradiated Nalm6. Additionally, most of the dual CAR T cells express only one or two exhaustion markers (PD-1 and/or CD39, but not Tim3 or Lag3). Thus, the T cell expansion can be considered as activation but not exhaustion.

### Example 8: Adenoviral Delivery of CD19 Antigen Enhances Dual CAR T Cell Tumor Infiltration

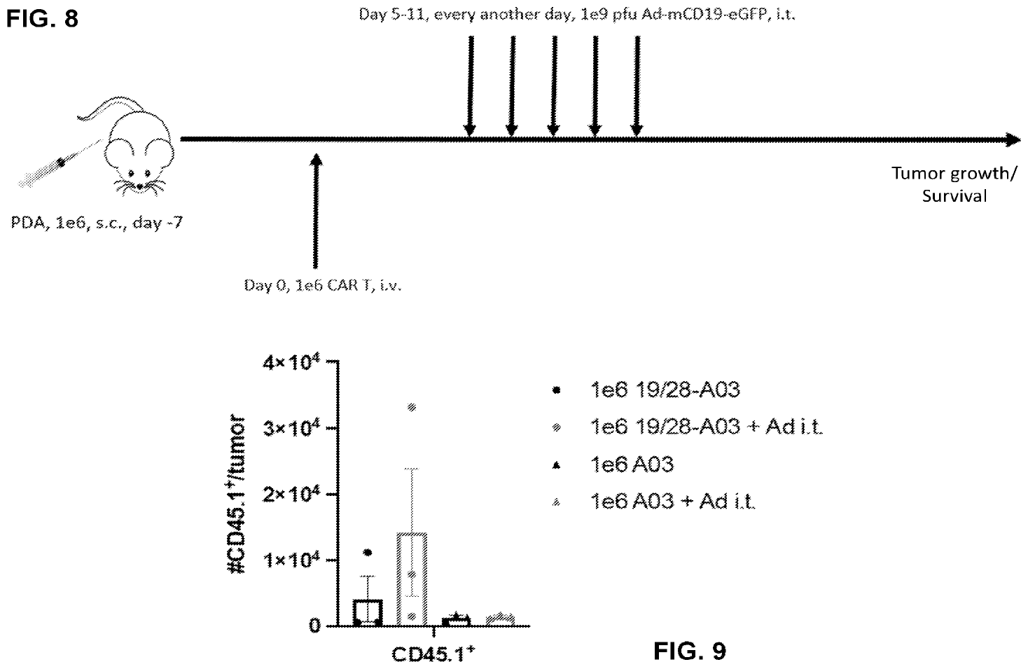

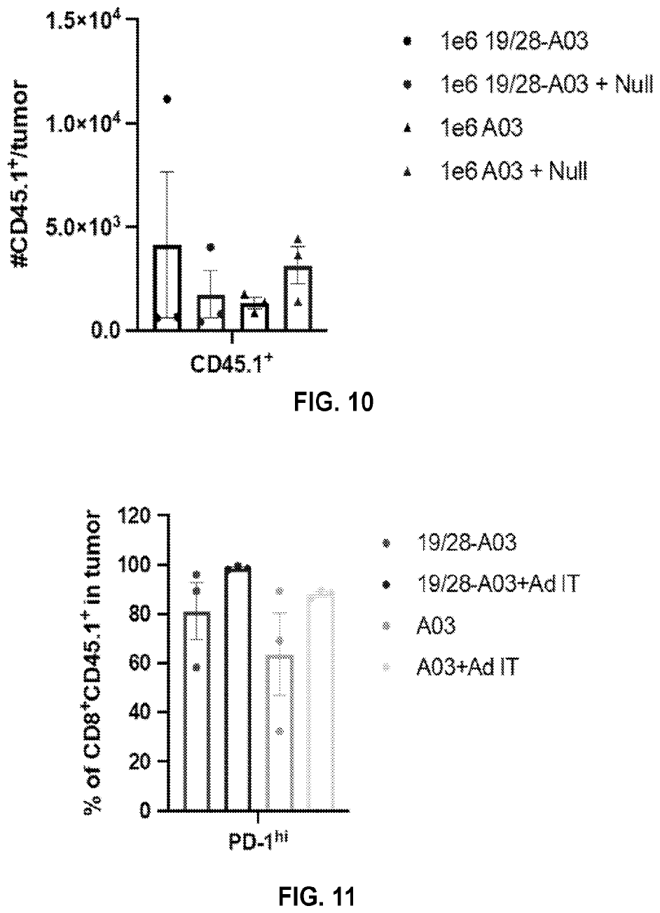

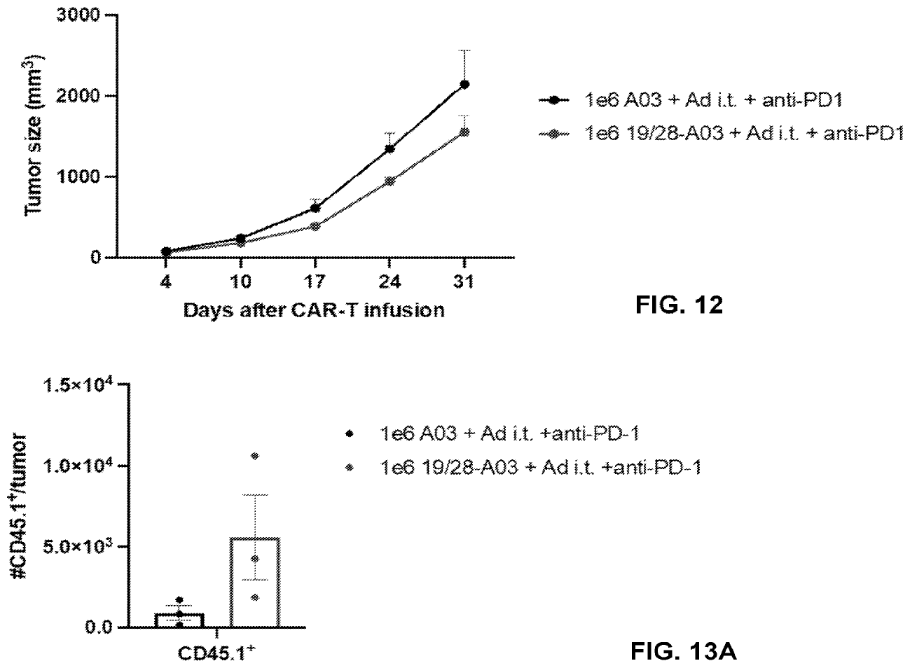

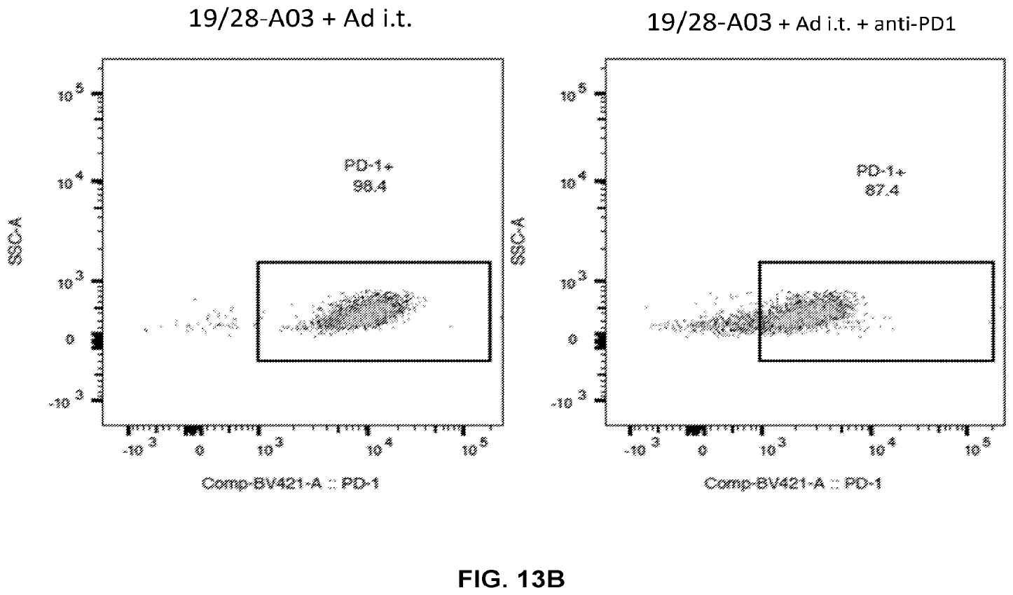

In order to increase CAR T cell trafficking and tumor infiltration, a replication-deficient E1/E3-deleted adenoviral vector encoding truncated CD19 antigen fused to eGFP (i.e., Ad-CMV-mCD19t-P2A-eGFP was administered intratumorally (IT) to C57BL/6 syngeneic mice after administration of dual CAR T cells (FIG. 8). Briefly, C57BL/6 syngeneic mice were implanted with PDA (pancreatic) tumors at day −7. On day 0, 1e6 CD45.1+ CAR T cells (transduced with either the MSGV-anti-moCD19-MuCD28z-anti-moMeso-A03-3-MuBBz dual CAR construct (“19/28z-A03” or with the MSGV-anti-moMeso-A03-3-MuBBz single CAR construct (“A03”)) were infused into the mice. Mice received 1e9 pfu Ad-CMV-mCD19t-P2A-eGFP every other day from day 5 to day 11. Negative control groups included mice which received CAR T cells transduced with the single anti-murine mesothelin (A03) CAR, mice which received a null adenoviral vector, mice which did not receive any adenoviral vector, and combinations thereof. On day 11 post CAR-T infusion, tumors were removed and characterized for CD45.1+ expression by flow cytometry (FIG. 9 and FIG. 10). A significant increase in CAR T cell infiltration into the tumor was observed in mice which received the adenoviral vector encoding CD19 antigen (FIG. 9) but not for control group mice (FIG. 9 and FIG. 10). Administration of adenoviral-expressed CD19 antigen likely increased TIL infiltration by two mechanisms: inducing tumor inflammation and supplying CD19 antigen. Indeed, administration of Ad alone (Ad-null) was not sufficient to achieve the same effect (FIG. 10). Although IT injection of Ad-CMV-mCD19t-P2A-eGFP significantly increased CAR-T infiltration, the CAR T cells did not display tumor control and expressed increased levels of PD-1 (FIG. 11). An immune-suppressive tumor microenvironment (TME) recapitulated in the syngeneic PDA model is a known barrier to effective CAR T cell therapy in solid cancers. Therefore, the therapy regimen was next combined with an anti-PD1 checkpoint blockade. Administration of anti-PD1 antibody together with CD19 antigen (as Ad-CMV-mCD19t-P2A-eGFP) resulted in a reduction in tumor size and an increase in tumor TIL infiltration for mice which received dual CAR T cells compared to mice which received CAR T cell expressing only the single A03 CAR (FIG. 12 and FIG. 13A). As expected, anti-PD1 antibody decreased the percentage and MFI of PD-1-expression in the mice which received dual CAR T cells and CD19 antigen (FIG. 13B).

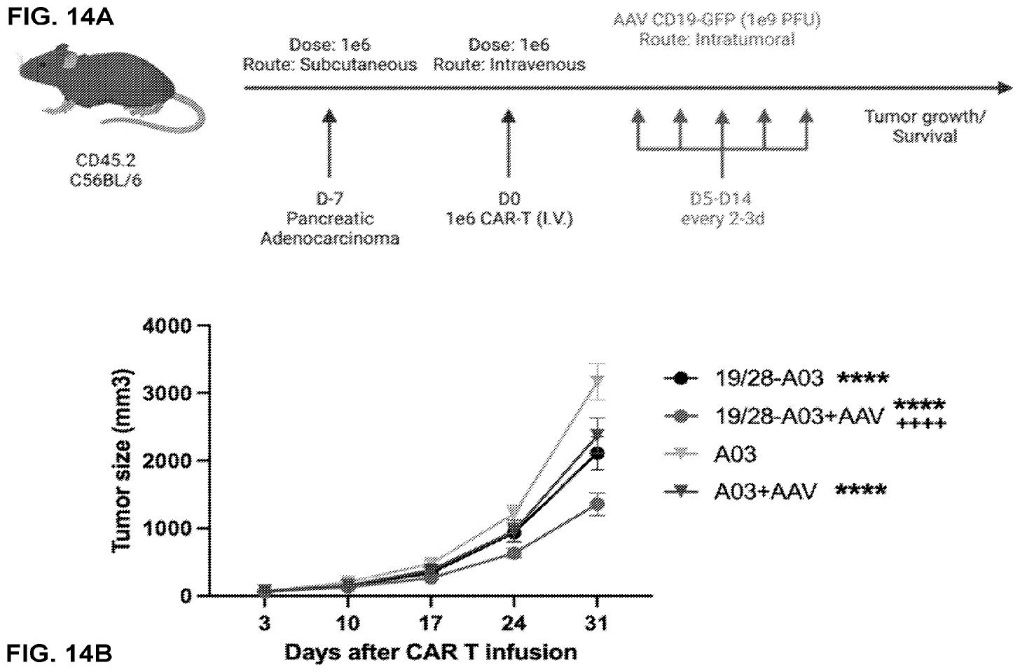

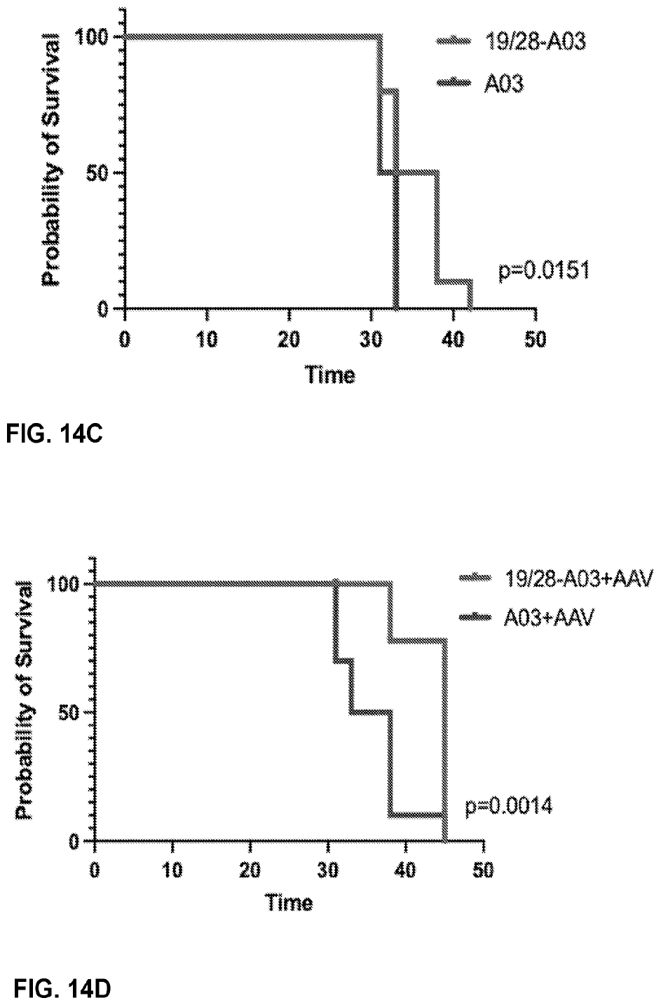

Next, mice were injected with mCAR T cells 7 days after “hot” PDA tumor engraftment and intermittently treated with adenovirus encoding truncated CD19 (FIG. 14A). Dual mCAR T cells demonstrate enhanced tumor burden control (FIG. 14B) and overall survival (FIGS. 14C-14D) against the immunologically “hot” pancreatic ductal adenocarcinoma (PDA), 2838c3, compared to single CAR T cells in the presence and absence of intertumoral truncated CD19 adenovirus treatment.

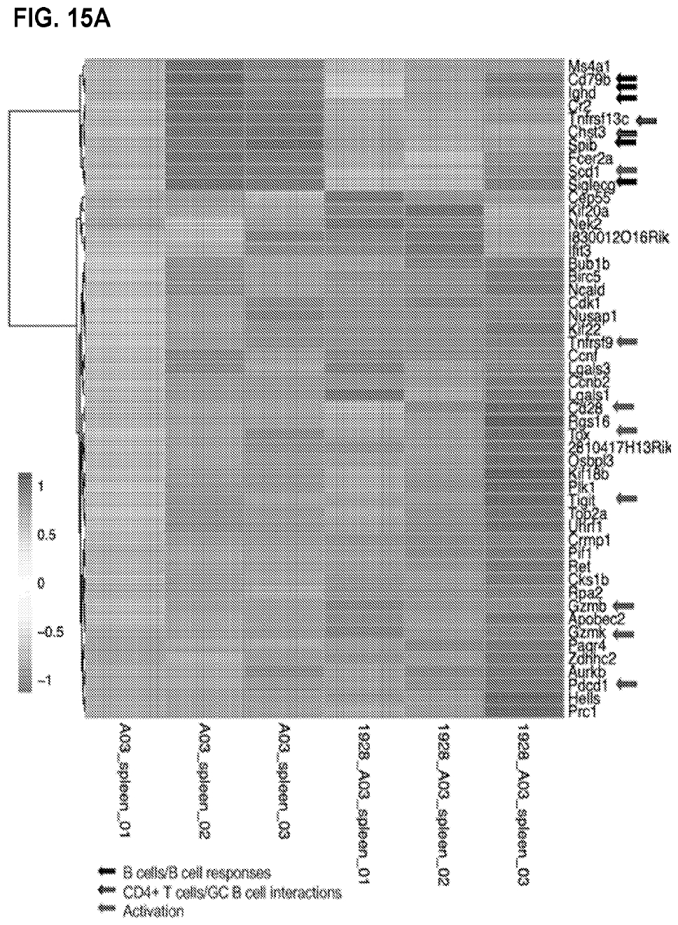

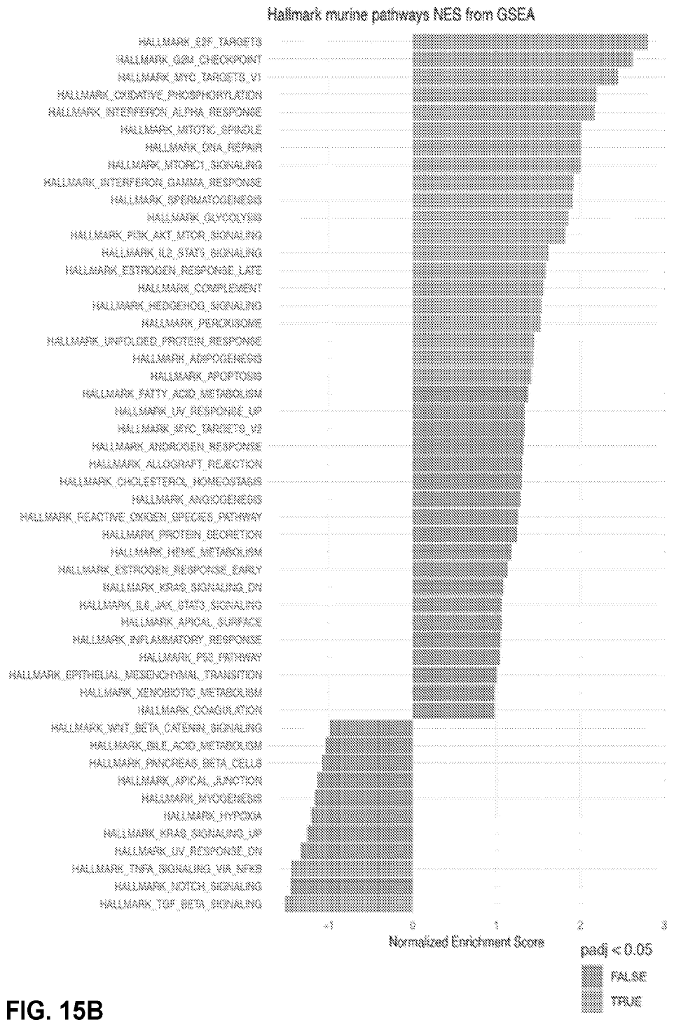

Computational analysis of the spleen following “cold” PDA tumor challenge revealed boosting in the spleen (FIGS. 15A-15B). Adapting the same experimental layout described above and shown in FIG. 14A with a “cold” tumor PDA model, CD45.1+ CAR T cells from the spleen were characterized 6 days after CAR T infusion via bulk RNA-seq. The data were normalized and analyzed using DESeq2 and the top differentially expressed genes were plotted (FIG. 15A). The fGSEA package was utilized to run gene set enrichment analysis (FIG. 15B). The observed transcriptional changes were consistent with increased activation of T cells and B cell aplasia within the spleens, such as the downregulation of B cell genes and genes associated with CD4+ T cell and GC B cell interactions in the Dual targeted CAR T cell cohort. Dual CAR T cells also demonstrate a high activation score, as demonstrated by the upregulation of genes associated with T cell activation, such as CD28, Tigit, and PD1 (FIG. 15A). This is also complemented by an upregulation of pathways associated with proliferation, metabolism, activation, and IFNγ signaling, without the upregulation of exhaustion signatures (FIG. 15B).

### Enumerated Embodiments

The following enumerated embodiments are provided, the numbering of which is not to be construed as designating levels of importance.

Embodiment 1 provides an isolated nucleic acid comprising:
a. a first nucleotide sequence encoding a first chimeric antigen receptor (CAR) comprising:
   (i) a first extracellular antigen binding domain having affinity for CD19,
   (ii) a first transmembrane domain, and
   (iii) a first intracellular domain comprising at least one costimulatory domain and a signaling domain; and
b. a second nucleotide sequence encoding a second CAR comprising:
   (i) a second extracellular antigen binding domain having affinity for a tumor antigen, wherein the tumor antigen is not CD19,
   (ii) a second transmembrane domain, and
   (iii) a second intracellular domain comprising at least one costimulatory domain and a signaling domain.

Embodiment 2 provides the isolated nucleic acid of embodiment 1, wherein the first nucleotide sequence is situated 5′ to the second nucleotide sequence or wherein the first nucleotide sequence is situated 3′ to the second nucleotide sequence.

Embodiment 3 provides the isolated nucleic acid of embodiment 1 or embodiment 2, further comprising a linker nucleotide sequence situated between the first nucleotide sequence and the second nucleotide sequence, wherein the linker nucleotide sequence comprises a ribosome slip sequence selected from the group consisting of P2A, T2A, E2A, and F2A.

Embodiment 4 provides the isolated nucleic acid of any one of the preceding embodiments, further comprising a promoter operably linked to the first nucleotide sequence and/or to the second nucleotide sequence.

Embodiment 5 provides the isolated nucleic acid of embodiment 4, wherein the promoter is an EF1α promoter.

Embodiment 6 provides the isolated nucleic acid of any one of the preceding embodiments, further comprising a posttranscriptional regulatory element.

Embodiment 7 provides the isolated nucleic acid of embodiment 6, wherein the posttranscriptional regulatory element is a woodchuck hepatitis virus (WHV) posttranscriptional regulatory element (WPRE).

Embodiment 8 provides the isolated nucleic acid of any one of the preceding embodiments, wherein the tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), Glycosyl-phosphatidylinositol (GPI)-linked GDNF family α-receptor 4 (GFRα4; GFRalpha4), HER2, HLA-A2, ICAM1, interleukin 13 receptor subunit alpha (IL3Rα), interleukin 13 receptor subunit alpha 1 (IL13Rα1), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), VEGFR2, and any combination thereof.

Embodiment 9 provides the isolated nucleic acid of any one of the preceding embodiments, wherein the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family α-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

Embodiment 10 provides the isolated nucleic acid of any one of the preceding embodiments, wherein the first extracellular antigen binding domain and the second extracellular antigen binding domain are each independently selected from a single-chain variable fragment (scFv) and an Fab.

Embodiment 11 provides the isolated nucleic acid of any one of the preceding embodiments, wherein the at least one costimulatory domain of the first or second intracellular domain comprises a costimulatory domain of a protein independently selected from 4-1BB, CD2, CD28, and ICOS.

Embodiment 12 provides the isolated nucleic acid of any one of the preceding embodiments, wherein the signaling domain comprises a CD3 zeta signaling domain.

Embodiment 13 provides the isolated nucleic acid of any one of the preceding embodiments, wherein the first and/or second transmembrane domain comprises a transmembrane domain of a protein independently selected from CD8, CD28, and 4-1BB.

Embodiment 14 provides the isolated nucleic acid of any one of the preceding embodiments, wherein the first and/or second CAR further comprises a hinge domain.

Embodiment 15 provides the isolated nucleic acid of embodiment 14, wherein the hinge domain comprises a CD8 hinge domain.

Embodiment 16 provides the isolated nucleic acid of any one of the preceding embodiments, wherein
a. the first CAR comprises:
   (i) a first extracellular antigen binding domain comprising an anti-CD19 scFv,
   (ii) a first transmembrane domain, and
   (iii) a first intracellular domain comprising a CD28 costimulatory domain and a CD3 zeta signaling domain; and
b. the second CAR comprises:
   (i) a second extracellular antigen binding domain comprising an anti-tumor antigen scFv, wherein the tumor antigen is not CD19,
   (ii) a second transmembrane domain, and
   (iii) a second intracellular domain comprising a 4-1BB costimulatory domain and a CD3 zeta signaling domain.

Embodiment 17 provides the isolated nucleic acid of any one of the preceding embodiments, wherein:
a. the first CAR consists essentially of
   (i) a CD8 leader sequence;
   (ii) a first extracellular antigen binding domain comprising an anti-CD19 scFv,
   (iii) a CD8 hinge;
   (iv) a CD28 transmembrane domain, and
   (v) a first intracellular domain comprising a CD28 costimulatory domain and a CD3 zeta signaling domain; and
b. the second CAR consists essentially of:
   (i) a CD8 leader sequence;
   (ii) a second extracellular antigen binding domain comprising an anti-mesothelin scFv,
   (iii) a CD8 hinge;
   (iv) a CD8 transmembrane domain, and
   (v) a second intracellular domain comprising a 4-1BB costimulatory domain and a CD3 zeta signaling domain.

Embodiment 18 provides the isolated nucleic acid of any one of the preceding embodiments, further comprising a third nucleotide sequence encoding a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, IFNγ and CD28, IFNγ and OX40, IFNγ and IL2R, and IFNγ and IL12R.

Embodiment 19 provides the isolated nucleic acid of any one of the preceding embodiments, further comprising a fourth nucleotide sequence encoding a dominant negative receptor, wherein the dominant negative receptor is dnTGFβR.

Embodiment 20 provides a vector comprising the isolated nucleic acid of any one of the preceding embodiments.

Embodiment 21 provides the vector of embodiment 26, wherein the vector is a lentiviral vector or a retroviral vector.

Embodiment 22 provides a modified cell comprising the isolated nucleic acid of any one of embodiments 1-19 or the vector of any one of embodiments 20-21.

Embodiment 23 provides the modified cell of embodiment 22, wherein the cell is selected from a bacterial cell, a fungal cell, a yeast cell, an insect cell, an animal cell, a mammalian cell, and a human cell.

Embodiment 24 provides the modified cell of embodiment 23, wherein the cell is a mammalian cell or a human cell, and further wherein the cell is an immune cell or precursor cell thereof.

Embodiment 25 provides the modified cell of embodiment 24, wherein the immune cell is a T cell.

Embodiment 26 provides a modified cell, wherein the cell is an immune cell or precursor cell thereof, and wherein the cell is engineered to express:
a. a first chimeric antigen receptor (CAR) comprising:
   (i) a first extracellular antigen binding domain having affinity for CD19,
   (ii) a first transmembrane domain, and
   (iii) a first intracellular domain comprising at least one costimulatory domain and a signaling domain; and
b. a second CAR comprising:
   (i) a second extracellular antigen binding domain having affinity for a tumor antigen, wherein the tumor antigen is not CD19,
   (ii) a second transmembrane domain, and
   (iii) a second intracellular domain comprising at least one costimulatory domain and a signaling domain.

Embodiment 27 provides the modified cell of embodiment 26, wherein the tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), HER2, HLA-A2, ICAM1, interleukin 13 receptor subunit alpha (IL3Rα), interleukin 13 receptor subunit alpha 1 (IL13Rα1), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), Glycosyl-phosphatidylinositol (GPI)-linked GDNF family α-receptor 4 (GFRα4; GFRalpha4), VEGFR2, and any combination thereof.

Embodiment 28 provides the modified cell of embodiment 26 or embodiment 27, wherein the tumor antigen is selected from the group consisting of prostate specific membrane antigen (PSMA), MUC1, Tn-glycoform of MUC1 (TnMUC1), folate receptor alpha (FRα), mesothelin, New York esophageal squamous cell carcinoma-1 (NY-ESO-1), glypican 2 (GPC2), Glycosyl-phosphatidylinositol (GPI)-linked GDNF family α-receptor 4 (GFRα4; GFRalpha4), prostate stem cell antigen (PSCA), fibroblast activation protein (FAP), epidermal growth factor receptor (EGFR), and interleukin 13 receptor subunit alpha 2 (IL13Rα2).

Embodiment 29 provides the modified cell of any one of the preceding embodiments, wherein the first extracellular antigen binding domain and the second extracellular antigen binding domain are each independently selected from a single-chain variable fragment (scFv) and an Fab.

Embodiment 30 provides the modified cell of any one of the preceding embodiments, wherein the at least one costimulatory domain of the first or second intracellular domain comprises a costimulatory domain of a protein independently selected from 4-1BB, CD2, CD28, and ICOS.

Embodiment 31 provides the modified cell of any one of the preceding embodiments, wherein the signaling domain comprises a CD3 zeta signaling domain.

Embodiment 32 provides the modified cell of any one of the preceding embodiments, wherein the first and/or second transmembrane domain comprises a transmembrane domain of a protein independently selected from CD8, CD28, and 4-1BB.

Embodiment 33 provides the modified cell of any one of the preceding embodiments, wherein the first and/or second CAR further comprises a hinge domain.

Embodiment 34 provides the modified cell of embodiment 33, wherein the hinge domain comprises a CD8 hinge domain.

Embodiment 35 provides the modified cell of any one of the preceding embodiments, wherein
a. the first CAR comprises:
   (i) a first extracellular antigen binding domain comprising an anti-CD19 scFv,
   (ii) a first transmembrane domain, and
   (iii) a first intracellular domain comprising a CD28 costimulatory domain and a CD3 zeta signaling domain; and
b. the second CAR comprises:
   (i) a second extracellular antigen binding domain comprising an anti-tumor antigen scFv, wherein the tumor antigen is not CD19,
   (ii) a second transmembrane domain, and
   (iii) a second intracellular domain comprising a 4-1BB costimulatory domain and a CD3 zeta signaling domain.

Embodiment 36 provides the modified cell of any one of the preceding embodiments, wherein
a. the first CAR consists essentially of
   (i) a CD8 leader sequence;
   (ii) a first extracellular antigen binding domain comprising an anti-CD19 scFv,
   (iii) a CD8 hinge;
   (iv) a CD28 transmembrane domain, and
   (v) a first intracellular domain comprising a CD28 costimulatory domain and a CD3 zeta signaling domain; and
b. the second CAR consists essentially of
   (i) a CD8 leader sequence;
   (ii) a second extracellular antigen binding domain comprising an anti-mesothelin scFv,
   (iii) a CD8 hinge;
   (iv) a CD8 transmembrane domain, and
   (v) a second intracellular domain comprising a 4-1BB costimulatory domain and a CD3 zeta signaling domain.

Embodiment 37 provides the modified cell of any one of the preceding embodiments, wherein the cell is further engineered to express a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VISTA and IL-9R, TIGIT and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, IFNγ and CD28, IFNγ and OX40, IFNγ and IL2R, and IFNγ and IL12R.

Embodiment 38 provides the modified cell of any one of the preceding embodiments, wherein the cell is further engineered to express a dominant negative receptor, wherein the dominant negative receptor is dnTGFβR.

Embodiment 39 provides the modified cell of any one of the preceding embodiments, wherein the cell comprises a vector encoding the first CAR and the second CAR.

Embodiment 40 provides the modified cell of embodiment 39, wherein the vector is a lentiviral vector or a retroviral vector.

Embodiment 41 provides the modified cell of embodiment 39 or embodiment 40, wherein the vector further encodes the switch receptor of embodiment 37 and/or the dominant negative receptor of embodiment 38.

Embodiment 42 provides the modified cell of any one of the preceding embodiments, wherein the cell is a mouse cell or a human cell.

Embodiment 43 provides the modified cell of any one of the preceding embodiments, wherein the cell is a T cell.

Embodiment 44 provides a pharmaceutical composition comprising a population of the modified cell of any one of the preceding embodiments and at least one pharmaceutically acceptable carrier.

Embodiment 45 provides a method of treating cancer in a subject in need thereof, the method comprising administering to the subject the pharmaceutical composition of embodiment 43.

Embodiment 46 provides a method of treating cancer in a subject in need thereof, the method comprising administering to the subject a population of modified cells, wherein the cells are immune cells or precursor cells thereof, and wherein the cells are engineered to express:
a. a first chimeric antigen receptor (CAR) comprising:
   (i) a first extracellular antigen binding domain having affinity for CD19,
   (ii) a first transmembrane domain, and
   (iii) a first intracellular domain comprising at least one costimulatory domain and a signaling domain; and
b. a second CAR comprising:
   (i) a second extracellular antigen binding domain having affinity for a tumor antigen, wherein the tumor antigen is not CD19,
   (ii) a second transmembrane domain, and
   (iii) a second intracellular domain comprising at least one costimulatory domain and a signaling domain.

Embodiment 47 provides the method of embodiment 46, wherein the tumor antigen is selected from the group consisting of alpha feto-protein (AFP)/HLA-A2, AXL, B7-H3, BCMA, CA-1X, CD2, CD3, CD4, CD5, CD7, CD8, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, epidermal growth factor receptor (EGFR), EGFRvIII, EpCAM, EphA2, fibroblast activation protein (FAP), folate receptor alpha (FRa)/folate binding protein (FBP), GD-2, Glycolipid F77, glypican 2 (GPC2), glypican-3 (GPC3), HER2, HLA-A2, ICAM1, interleukin 13 receptor subunit alpha (IL3Rα), interleukin 13 receptor subunit alpha 2 (IL13Rα2), LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), New York esophageal squamous cell carcinoma-1 (NY-ESO-1), P16, PD-L1, prostate stem cell antigen (PSCA), prostate specific membrane antigen (PSMA), ROR1, ROR2, TIM-3, TM4SF1, Tn-glycoform of MUC1 (TnMUC1), Glycosyl-phosphatidylinositol (GPI)-linked GDNF family α-receptor 4 (GFRα4; GFRalpha4), VEGFR2, and any combination thereof.

Embodiment 48 provides the method of embodiment 46 or embodiment 47, wherein the tumor antigen is selected from the group consisting of prostate specific membrane antigen (PSMA), MUC1, Tn-glycoform of MUC1 (TnMUC1), folate receptor alpha (FRα), mesothelin, New York esophageal squamous cell carcinoma-1 (NY-ESO-1), glypican 2 (GPC2), Glycosyl-phosphatidylinositol (GPI)-linked GDNF family α-receptor 4 (GFRα4; GFRalpha4), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

Embodiment 49 provides the method of any one of the preceding embodiments, wherein the first extracellular antigen binding domain and the second extracellular antigen binding domain are each independently selected from a single-chain variable fragment (scFv) and an Fab.

Embodiment 50 provides the method of any one of the preceding embodiments, wherein the at least one costimulatory domain of the first or second intracellular domain comprises a costimulatory domain of a protein independently selected from 4-1BB, CD2, CD28, and ICOS.

Embodiment 51 provides the method of any one of the preceding embodiments, wherein the signaling domain comprises a CD3 zeta signaling domain.

Embodiment 52 provides the method of any one of the preceding embodiments, wherein the first and/or second transmembrane domain comprises a transmembrane domain of a protein independently selected from CD8, CD28, and 4-1BB.

Embodiment 53 provides the method of any one of the preceding embodiments, wherein the first and/or second CAR further comprises a hinge domain.

Embodiment 54 provides the method of embodiment 53, wherein the hinge domain comprises a CD8 hinge domain.

Embodiment 55 provides the method of any one of the preceding embodiments, wherein
a. the first CAR comprises:
   (i) a first extracellular antigen binding domain comprising an anti-CD19 scFv,
   (ii) a first transmembrane domain, and
   (iii) a first intracellular domain comprising a CD28 costimulatory domain and a CD3 zeta signaling domain; and
b. the second CAR comprises:
   (i) a second extracellular antigen binding domain comprising an anti-tumor antigen scFv, wherein the tumor antigen is not CD19,
   (ii) a second transmembrane domain, and
   (iii) a second intracellular domain comprising a 4-1BB costimulatory domain and a CD3 zeta signaling domain.

Embodiment 56 provides the method of any one of the preceding embodiments, wherein
a. the first CAR consists essentially of:
   (i) a CD8 leader sequence;
   (ii) a first extracellular antigen binding domain comprising an anti-CD19 scFv,
   (iii) a CD8 hinge;
   (iv) a CD28 transmembrane domain, and
   (v) a first intracellular domain comprising a CD28 costimulatory domain and a CD3 zeta signaling domain; and
b. the second CAR consists essentially of:
   (i) a CD8 leader sequence;
   (ii) a second extracellular antigen binding domain comprising an anti-mesothelin scFv,
   (iii) a CD8 hinge;
   (iv) a CD8 transmembrane domain, and
   (v) a second intracellular domain comprising a 4-1BB costimulatory domain and a CD3 zeta signaling domain.

Embodiment 57 provides the method of any one of the preceding embodiments, wherein the modified cells are further engineered to express a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VISTA and IL-9R, TIGIT and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, IFNγ and CD28, IFNγ and OX40, IFNγ and IL2R, and IFNγ and IL12R.

Embodiment 58 provides the method of any one of the preceding embodiments, wherein the modified cells are further engineered to express a dominant negative receptor, wherein the dominant negative receptor is dnTGFβR.

Embodiment 59 provides the method of any one of the preceding embodiments, wherein the modified cells comprise a vector encoding the first CAR and the second CAR.

Embodiment 60 provides the method of embodiment 59, wherein the vector is a lentiviral vector or a retroviral vector.

Embodiment 61 provides the method of embodiment 59 or embodiment 60, wherein the vector further encodes the switch receptor of embodiment 57 and/or the dominant negative receptor of embodiment 58.

Embodiment 62 provides the method of any one of the preceding embodiments, wherein the modified cells are mouse cells or human cells.

Embodiment 63 provides the method of any one of the preceding embodiments, wherein the population of modified cells comprises T cells.

Embodiment 64 provides the method of any one of the preceding embodiments, wherein the modified cells are autologous to the subject.

Embodiment 65 provides the method of any one of the preceding embodiments, wherein the modified cells are allogeneic to the subject.

Embodiment 66 provides the method of any one of the preceding embodiments, wherein the population of modified cells are administered as a pharmaceutical composition comprising the population of modified cells and at least one pharmaceutically acceptable carrier.

Embodiment 67 provides the method of any one of the preceding embodiments, wherein the population of modified cells comprises about 1×10⁶ to about 1×10⁹ cells.

Embodiment 68 provides the method of any one of the preceding embodiments, wherein the modified cells exhibit expansion in peripheral blood of the subject.

Embodiment 69 provides the method of embodiment 68, wherein the expansion is at least 10-fold, at least 100-fold, or at least 1000-fold.

Embodiment 70 provides the method of any one of the preceding embodiments, wherein the modified cells are detectable for at least 24 months after administering the cells.

Embodiment 71 provides the method of any one of the preceding embodiments, wherein the subject is a human.

Embodiment 72 provides the method of any one of the preceding embodiments, wherein the cancer is selected from breast cancer, prostate cancer, ovarian cancer, cervical cancer, skin cancer, prostate cancer, pancreatic cancer, colorectal cancer, renal cancer, liver cancer, brain cancer, lymphoma, leukemia, lung cancer, and thyroid cancer.

Embodiment 73 provides the method of any one of the preceding embodiments, wherein the population of modified cells comprises T cells and at least 30% or at least 40% of the population of modified cells at day 7 post-administration or beyond are phenotypically central memory T cells.

Embodiment 74 provides the method of any one of the preceding embodiments, wherein the method further comprises administering a CD19 antigen to the subject.

Embodiment 75 provides the method of embodiment 74, wherein administering the CD19 antigen comprises administering a vector encoding the CD19 antigen or a cell engineered to express the CD19 antigen.

Embodiment 76 provides the method of embodiment 74 or 75, wherein the CD19 antigen comprises a CD19 extracellular domain or antigenic fragment thereof.

Embodiment 77 provides the method of any one of embodiments 74-76, wherein the CD19 antigen is administered prior to, concurrently with, or after the administration of the population of modified cells.

Embodiment 78 provides the method of any one of embodiments 75-77, wherein the vector is an adenoviral vector.

Embodiment 79 provides the method of any one of embodiments 74-78, wherein the method further comprises administering an anti-PD1 immunotherapy to the subject.

Embodiment 80 provides the method of embodiment 79, wherein the anti-PD1 immunotherapy is an anti-PD1 antibody.

### OTHER EMBODIMENTS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A nucleic acid comprising:
a. a first nucleotide sequence encoding a first chimeric antigen receptor (CAR) comprising:
(i) a first extracellular antigen binding domain having affinity for CD19,
(ii) a first transmembrane domain, and
(iii) a first intracellular domain comprising at least one costimulatory domain and a signaling domain; and
b. a second nucleotide sequence encoding a second CAR comprising:
(i) a second extracellular antigen binding domain having affinity for a tumor antigen, wherein the tumor antigen is not CD19,
(ii) a second transmembrane domain, and
(iii) a second intracellular domain comprising at least one costimulatory domain and a signaling domain.

2-8. (canceled)

9. The nucleic acid of claim 1, wherein the tumor antigen is selected from the group consisting of prostate specific membrane antigen (PSMA), MUC1, Tn-glycoform of MUC1 (TnMUC1), Glycosyl-phosphatidylinositol (GPI)-linked GDNF family a-receptor 4 (GFRα4; GFRalpha4), folate receptor alpha (FRα), mesothelin, New York esophageal squamous cell carcinoma-1 (NY-ESO-1), glypican 2 (GPC2), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

10-17. (canceled)

18. The nucleic acid of claim 1, further comprising a third nucleotide sequence encoding a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R.

19. The nucleic acid of claim 1, further comprising a fourth nucleotide sequence encoding a dominant negative receptor, wherein the dominant negative receptor is dnTGFβR.

20. A vector comprising the nucleic acid of claim 1, optionally, wherein the vector is a lentiviral vector or a retroviral vector.

21-25. (canceled)

26. A modified cell, wherein the cell is an immune cell or precursor cell thereof, and wherein the cell is engineered to express:
a. a first chimeric antigen receptor (CAR) comprising:
(i) a first extracellular antigen binding domain having affinity for CD19,
(ii) a first transmembrane domain, and
(iii) a first intracellular domain comprising at least one costimulatory domain and a signaling domain; and
b. a second CAR comprising:
(i) a second extracellular antigen binding domain having affinity for a tumor antigen, wherein the tumor antigen is not CD19,
(ii) a second transmembrane domain, and
(iii) a second intracellular domain comprising at least one costimulatory domain and a signaling domain.

27. (canceled)

28. The modified cell of claim 26, wherein the tumor antigen is selected from the group consisting of prostate specific membrane antigen (PSMA), MUC1, Tn-glycoform of MUC1 (TnMUC1), folate receptor alpha (FRα), mesothelin, New York esophageal squamous cell carcinoma-1 (NY-ESO-1), glypican 2 (GPC2), Glycosyl-phosphatidylinositol (GPI)-linked GDNF family α-receptor 4 (GFRα4; GFRalpha4), prostate stem cell antigen (PSCA), fibroblast activation protein (FAP), epidermal growth factor receptor (EGFR), and interleukin 13 receptor subunit alpha 2 (IL13Rα2).

29-36. (canceled)

37. The modified cell of claim 26, wherein the cell is further engineered to express a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R.

38. The modified cell of claim 26, wherein the cell is further engineered to express a dominant negative receptor, wherein the dominant negative receptor is dnTGFβR.

39-42. (canceled)

43. The modified cell of claim 26, wherein the cell is a T cell.

44. A pharmaceutical composition comprising a population of the modified cell of claim 26 and at least one pharmaceutically acceptable carrier.

45. (canceled)

46. A method of treating cancer in a subject in need thereof, the method comprising administering to the subject a population of modified cells, wherein the cells are immune cells or precursor cells thereof, and wherein the cells are engineered to express:
a. a first chimeric antigen receptor (CAR) comprising:
(i) a first extracellular antigen binding domain having affinity for CD19,
(ii) a first transmembrane domain, and
(iii) a first intracellular domain comprising at least one costimulatory domain and a signaling domain; and
b. a second CAR comprising:
(i) a second extracellular antigen binding domain having affinity for a tumor antigen, wherein the tumor antigen is not CD19,
(ii) a second transmembrane domain, and
(iii) a second intracellular domain comprising at least one costimulatory domain and a signaling domain.

47. (canceled)

48. The method of claim 46, wherein the tumor antigen is selected from the group consisting of prostate specific membrane antigen (PSMA), MUC1, Tn-glycoform of MUC1 (TnMUC1), folate receptor alpha (FRα), mesothelin, New York esophageal squamous cell carcinoma-1 (NY-ESO-1), glypican 2 (GPC2), Glycosyl-phosphatidylinositol (GPI)-linked GDNF family α-receptor 4 (GFRα4; GFRalpha4), prostate stem cell antigen (PSCA), fibroblast activation protein (FAP), epidermal growth factor receptor (EGFR), interleukin 13 receptor subunit alpha 1 (IL13Rα1), and interleukin 13 receptor subunit alpha 2 (IL13Rα2).

49-56. (canceled)

57. The method of claim 46, wherein the modified cells are further engineered to express a switch receptor, wherein the switch receptor comprises an extracellular domain of a first receptor and an intracellular domain of a second receptor, wherein the first receptor and the second receptor, respectively, are selected from the group consisting of TGFβR and IL12R, TGFβR and CD28, TGFβR and OX40, TGFβR and CD27, TGFβR and 4-1BB, TGFβR and IL-2R, TGFβR and IL-9R, PD1 and IL12R, PD1 and CD28, PD1 and ICOS, PD1 and CD27, PD1 and 4-1BB, PD1 and IL-2R, PD1 and IL-9R, BTLA and CD28, BTLA and ICOS, BTLA and CD27, CTLA4 and CD28,TIM3 and IL12R, TIM3 and CD28, TIM3 and CD28, TIM3 and OX40, TIM3 and 4-1BB, TIM3 and IL-2R, TIM3 and IL-9R, VSIG3 and CD28, VSIG3-IL12Rβ2, VSIG3 and CD27, VSIG3 and 4-1BB, VSIG3 and ICOS, VSIG3-IL12Rβ1, VSIG8-IL12Rβ1, VSIG8 and CD28, VSIG8-IL12Rβ2, VSIG8 and CD27, VSIG8 and 4-1BB, VSIG8-ICOS, VISTA and IL-9R, TIGIT and IL-9R, IFNγ and CD28, IFNγ and OX40, IFNγ and IL2R, and IFNγ and IL12R.

58. The method of claim 46, wherein the modified cells are further engineered to express a dominant negative receptor, wherein the dominant negative receptor is dnTGFβR.

59-62. (canceled)

63. The method of claim 46, wherein the population of modified cells comprises T cells.

64-67. (canceled)

68. The method of claim 46, wherein the modified cells exhibit expansion in peripheral blood of the subject, further wherein the expansion is at least 10-fold, at least 100-fold, or at least 1000-fold.

69. (canceled)

70. The method of claim 46, wherein the modified cells are detectable for at least 24 months after administering the cells.

71-72. (canceled)

73. The method of claim 46, wherein the population of modified cells comprises T cells and at least 30% or at least 40% of the population of modified cells at day 7 post-administration or beyond are phenotypically central memory T cells.

74. The method of claim 46, wherein the method further comprises administering a CD19 antigen to the subject, further wherein:
(i) administering the CD19 antigen comprises administering a vector encoding the CD19 antigen or a cell engineered to express the CD19 antigen, optionally wherein the vector is an adenoviral vector;
(ii) the CD19 antigen comprises a CD19 extracellular domain or antigenic fragment thereof; and/or
(iii) the CD19 antigen is administered prior to, concurrently with, or after the administration of the population of modified cells.

75-78. (canceled)

79. The method of claim 74, wherein the method further comprises administering an anti-PD1 immunotherapy to the subject, optionally wherein the anti-PD1 immunotherapy is an anti-PD1 antibody.

80. (canceled)
